(19) United States
(12) Patent Application Publication
(10) Pub. No.: US 2025/0305056 A1
(43) Pub. Date: Oct. 2, 2025

(54) KITS AND METHODS FOR DETERMINATION OF CLL MUTATIONAL STATUS

(71) Applicants: Assistance Publique - Hopitaux de Paris, Paris (FR); Sorbonne Universite, Paris (FR); INSERM (Institut National de la Santé et de la Recherche Médicale), Paris (FR)

(72) Inventors: Frédéric DAVI, Meudon (FR); Nour el Houda BOUDJOGHRA, Romans Sur Isere (FR); Anne LANGLOIS DE SEPTENVILLE, Montrouge (FR)

(73) Assignees: Assistance Publique - Hopitaux de Paris, Paris (FR); Sorbonne Universite, Paris (FR); INSERM (Institut National de la Santé et de la Recherche Médicale), Paris (FR)

(21) Appl. No.: 18/722,943

(22) PCT Filed: Dec. 22, 2022
(86) PCT No.: PCT/EP2022/087507

§ 371 Date: Jun. 21, 2024

(30) Foreign Application Priority Data
Dec. 23, 2021 (EP) 21306914.9

(51) Int. Cl. C12Q 1/6886

(52) U.S. Cl. CPC C12Q 1/6886; C12Q 2600/156; C12Q 2600/16

(57) ABSTRACT

The present invention relates to kits for determining the mutational status of a patient suffering from CLL from gDNA or cDNA extracted from a biological sample of said patient by NGS or Sanger sequencing, comprising forward and reverse amplification primers, and optionally an internal control containing a mixture of nucleic acid molecules encoding productive clonal IGH rearrangement representative of all IGHV segments. It further relates to methods for determining the mutational status of a patient suffering from CLL from a biological sample of said CLL patient using such kits. Such kits and methods are useful for the management of CLL, and especially of the prognosis and treatment choice of CLL patients.

### CROSS-REFERENCE TO RELATED APPLICATIONS

This application is a U.S. National Stage Application pursuant to 35 U.S.C. § 371 of International Patent Application PCT/EP2022/087507, filed on Dec. 22, 2022, and published as WO 2023/118452 on Jun. 29, 2023, which claims priority to European Patent Application No. 21306914.9, filed on Dec. 23, 2021, all of which are incorporated herein by reference in their entireties for all purposes.

### REFERENCE TO SEQUENCE LISTING SUBMITTED ELECTRONICALLY

The Sequence Listing associated with this application is provided in XML format, and is hereby incorporated by reference into the specification. The name of the XML file containing the Sequence Listing is “Sequence Listing filed.” The XML file is 147,456 bytes, was created on Jun. 21, 2024, and is being submitted electronically, concurrent with the filing of the specification.

### TECHNICAL FIELD OF THE INVENTION

The present invention is in the field of the management of B-cell chronic lymphocytic leukemia (CLL), and especially of the prognosis and prediction of response to treatment of CLL patients. It relates to kits for determining the mutational status of a patient suffering from CLL from genomic DNA (gDNA) or complementary DNA (cDNA) extracted from a biological sample of said patient by NGS or Sanger sequencing, comprising forward and reverse amplification primers, and optionally an internal control containing a mixture of plasmids encoding productive clonal IGH rearrangement representative of all IGHV functional genes. It further relates to methods for determining the mutational status of a patient suffering from CLL from a biological sample of said CLL patient using such kits.

### BACKGROUND ART

Chronic lymphocytic leukemia (CLL) is the most common form of leukemia among adults in the Western world. It affects mainly middle-aged and elderly individuals, with a median age at diagnosis ranging from 67 to 72 years (Hallek M, et al. Lancet. 2018; 391(10129): 1524-1537). It is characterized by the proliferation and accumulation of monoclonal B cells in the blood, bone marrow and lymphoid organs. The diagnosis is based on immunophenotyping of leukemic cells which express a typical antigenic profile, including co-expression of CD19, CD5, and CD23 coupled with low levels of surface immunoglobulins (Moreau E J et al. Am J Clin Pathol. 1997; 108(4): 378-82). CLL has highly variable clinical course, with some patients experiencing a rapidly progressing form requiring early treatment, while others have a very indolent disease which may not necessitate therapy for many years, if at all (Hallek M, et al. Lancet. 2018; 391(10129): 1524-1537). It is also recommended in the European Society for Medical Oncology (ESMO) clinical practice guidelines for diagnosis, treatment and follow-up of CLL (Eichhorst B et al. Ann. Oncol. 2021; 32(1):23-33) and endorsed by the European Society of Hematology (Eichhorst B, Ghia P. HemaSphere. 2020; 5(1):e520).

In the past two decades, advances have been made to understand the basis of this heterogeneity and several biomarkers serving as prognosis indicators have been identified. Among them, the mutational status of the immunoglobulin heavy chain variable region (IGHV) genes, has emerged has one of the most robust prognostic markers, as patients with unmutated IGHV genes have a more aggressive disease course than patients with mutated IGHV genes (Damle R N, et al. Blood. 1999; 94(6):1840-7; Hamblin T J, et al. Blood. 1999; 94(6):1848-54). Importantly, it is independent of clinical stage or other biomarkers, and has the advantage of being identifiable at diagnosis and to remain stable over time (Sutton L A, et al. Haematologica. 2017; 102(6):968-971). Furthermore, the IGHV mutational status has also proven to have a strong predictive value for response to treatment, distinguishing patients who benefit from chemoimmunotherapy regimens (mutated IGHV genes) from those who will require targeted therapies such as BTK inhibitors (unmutated IGHV genes) (Chai-Adisaksopha C, Brown J R. Blood. 2017; 130(21):2278-2282). Therefore, determination of the IGHV genes mutational status is now recommended prior treatment initiation according to the most recent International Workshop on CLL guidelines (Hallek M, et al. Blood. 2018; 131(25):2745-2760).

The B-cell receptor (BcR) is an essential component expressed on the surface of all normal and malignant B cells (see FIG. 1). It is a complex formed by a transmembrane antigen-recognition unit, the immunoglobulin (abbreviated as “IG”), and a signaling unit (the intracellular-associated CD79a and CD79b molecules). IG is a heterodimer formed by 2 heavy (IGH) chains and 2 light (IGL) chains. Each chain comprises 2 parts: the variable (V) region, which recognizes and binds to target antigens, and the constant (C) region attached to the B cell's membrane. The extreme variability of the V regions, “matching” the huge diversity of antigens, results from complex genetics mechanisms. For IGH chains, the variable regions are encoded by 3 genes: IGHV (V for variable), IGHD (D for diversity) and IGHJ (J for joining). There is a potential reservoir of functional (or “useful”) 48-55 IGHV (as 7 genes are duplicated), 27 IGHD and 6 IGHJ genes (in addition there are also non-functional genes). The genes are separated on the genome and become juxtaposed during B-cell development. The process is (i) random, each gene having an equal probability of being “rearranged” (this 1^{st} type of diversity is referred to as “combinational diversity”), and (ii) imprecise, as a variable number of nucleotides are deleted and inserted at the IGHV-IGHD and IGHD-IGHJ junctions, thereby creating considerable diversity at the junction region called complementary determining region 3 (CDR3) (this 2^{nd} type of diversity is referred to as “junctional diversity”). Of note the same process occurs for the IGL chains with the difference that there are no D genes. Altogether this genetic process, called VDJ recombination, leads to an extreme diversity in the V regions of IG (Schatz D G. V(D)J recombination. Immunol Rev. 2004; 200:5-11).

In B cells, another mechanism further increases this diversity. After a first encounter with an antigen, the V regions undergo a high number of nucleotides changes, a process called somatic hypermutation (abbreviated as “SHM”), allowing the IG to acquire a better affinity for their cognate antigens (this 3^{rd} type of diversity is referred to as “SHM diversity”). In addition, this can be associated (but not systematically) with a change of constant region switching from native M-type (and D-type) to G-type, A-type or rarely E-type. The vast majority of CLLs express IgM or IgM+IgD molecules, and a minority (≈10%) IgG.

CLL are heterogeneous regarding the SHM process. Some have no or few mutations and are called “unmutated” (abbreviated as “U-CLL”), while others have a substantial number of mutations and are called “mutated” (abbreviated as “M-CLL”). As indicated above, this has major consequences in term of clinical behavior of the disease, with M-CLLs having a much better prognosis than U-CLLs. The consensus threshold between these 2 categories is the presence 2% of mutations within the IGHV gene: thus U-CLL correspond to CLL with 2% or less of mutations within the IGHV gene, while M-CLL correspond to CLL with more than 2% of mutations within the IGHV gene (Damle R N, et al. Blood. 1999; 94(6):1840-7; Hamblin T J, et al. Blood. 1999; 94(6):1848-54). The mutational load is determined by comparing the sequence of the CLL IGHV gene with that of the ancestral germline gene from which it derives from VDJ recombination and counting all the variant nucleotides. This is done by submitting the IGH V region sequence to the universally acknowledged IMGT website IMGT/V-QUEST software (Brochet X, Lefranc M P, Giudicelli V. Nucleic Acids Res. 2008; 36(Web Server issue):W503-8) which (i) has the repository collection of all IG genes and alleles sequences, (ii) allows recognition and identification of the IGH-V, D and J genes within a V region, and (iii) calculates the % of identity of the IGHV gene with its with closest germline version (see FIG. 2).

The process of IGHV mutational status assessment includes several steps:
1) nucleic acids extraction from leukemic cells (blood, bone marrow, lymph node or other tissue): these can be either genomic DNA (gDNA) or RNA (thereafter transformed into complementary DNA (cDNA));
2) polymerase chain reaction (PCR) amplification of their V region;
3) sequencing of the PCR products using either next-generation sequencing (NGS) or conventional Sanger techniques; and
4) bioinformatic analysis of the sequence(s).

A critical step is the PCR amplification of the IG V region. This is accomplished typically by using 5′ primers annealing to the V gene and 3′ primers annealing to the J gene. In the case of CLL, for a proper % identity calculation, it is necessary to obtain sequence information from the entire IGHV. For this purpose, 5′ primers localized upstream the IGHV gene should be used, e.g. in a part coding for the so-called leader peptide (a short sequence which allows proper trafficking of the IG from the cytoplasm to the cell surface). For IGH, this leader peptide is encoded in 2 parts: a short stretch of nucleotides at the 5′ end of the IGHV (the L2-part) and an upstream exon (the L1-part) separated from the former by a ≈100 base pairs (bp) intron (see FIG. 1).

The possibilities regarding the 3′ primers depend on the type of nucleic acid template used for amplification. When starting from gDNA, they are located on the IGHJ gene, while with RNA/cDNA one can use the IGHC gene. As the C region is never targeted by SHM, this is a useful alternative when mutations in the IGHJ gene prevent primer annealing, which results in absence of amplification of the target. Of note, this strategy is not possible on gDNA as the genes coding for the constant region (IGHC) are located too far away from the IGH-VDJ rearrangement to allow PCR amplification. In contrast the IGHC gene is brought in contiguity to the IGHJ gene on the RNA molecule. However, many published methods for determination of CLL mutational status (STAMATOPOULOS K: BLOOD, vol. 106, no. 10, 15 Nov. 2005 (2005 Nov. 15), pages 3575-3583) and commercial kits (e.g. the “IGH hypermutation assay 2.0” available from Invivoscribe used in Stamatopoulos B. et al, LEUKEMIA, vol. 31, no. 4, 31 Oct. 2016 (2016 Oct. 31), pages 837-845) still rely only on 3′ primers located in the IGHJ region, no matter which type of sample (gDNA or cDNA) is used, which results in non-negligible number of failures when the IGHJ region is mutated.

A popular method for PCR amplification of the V region relies on the use of the Biomed-2 primers which have been designed for clonality assessment of lymphoid proliferations (van Dongen et al. Leukemia. 2003; 17(12):2257-317). However, these primers anneal to the FR1 region of the IGHV genes. The same applies to other published methods (K. STAMATOPOULOS K: BLOOD, vol. 106, no. 10, 15 Nov. 2005 (2005 Nov. 15), pages 3575-3583) and to commercial kits, including the “IGH hypermutation assay 2.0” available from Invivoscribe used in Stamatopoulos B. et al, LEUKEMIA, vol. 31, no. 4, 31 Oct. 2016 (2016 Oct. 31), pages 837-845), which include primers in the FR1 region. However, using primers in the FR1 region leads to an incomplete IGHV sequence with a risk of inaccurate mutational status assessment. This is why the European Research Initiative on CLL (ERIC) experts strongly recommend the use of peptide leader primers (Rosenquist R, et al. Leukemia. 2017; 31(7):1477-1481).

Unfortunately, the previously published primers proved suboptimal with a substantial failure rate (up to 24%) as shown when tested on a relatively large cohort of CLL patients (Huet 5, et al. Leukemia. 2020; 34(8):2257-2259).

Some guidelines and recommendations on how to perform IGHV mutational status determination have been published (Ghia P, et al. Leukemia. 2007; 21(1):1-3; Langerak aw et al. Leukemia. 2011; 25 (6), 979-984; Rosenquist R, et al. Leukemia. 2017; 31(7):1477-1481;) which have been cited in a recent review on this topic (Gupta Sanjeev Kumar et al: “Evaluation of Somatic Hypermutation Status in Chronic Lymphocytic Leukemia (CLL) in the Era of Next Generation Sequencing”, FRONTIERS IN CELL AND DEVELOPMENTAL BIOLOGY, vol. 8, 19 May 2020 (2020 May 19)), but they are based on individual authors experience only with Sanger sequencing and have not been “cross-validated” between laboratories. Furthermore, nowadays an increasing number of laboratories are switching to NGS for this biological test, but there are no standardized methodology available for this technology. As IGHV mutational status assessment is now mandatory for CLL patient care including treatment choice, there is a clear need for efficient, reliable and standardized, methodology ensuring accurate determination of this biomarker in every diagnostic laboratory.

### SUMMARY OF THE INVENTION

In the context of the present invention, the inventors have developed a methodology resulting in a very high rate of success in determination of the IGHV mutational status in CLL. The methodology has been validated by 4 collaborating laboratories, thereby demonstrating its robustness. It relies on PCR-based assays which allow the amplification of the entire IGHV regions, starting from gDNA and/or cDNA templates. The PCR products can be subsequently sequenced by either traditional Sanger or NGS techniques.

As PCR amplification of IGHV regions is the crucial point for reliable determination of CLL mutational status, they have designed different sets of primer combinations able to address distinct situations. For instance, the location and complete sequence of the primers is dependent on the type of sequencing methodology and the type of nucleic acid used. Indeed, NGS may be used only for sequencing relatively short nucleic acids (lower than about 500 bp). In addition, while IGHJ and IGHC genes are close to each other in cDNA, they are separated by a large intron in gDNA (the same is true for the L1 and L2 parts of the IGHV genes, although the intron is smaller).

As a result, forward and reverse primers were selected as follows depending on the type of DNA analyzed and the type of sequencing technique used (see also FIGS. 1 and 3-4):
(i) For NGS
   from gDNA: forward primers located on the L2 part of the IGHV genes (as shorter PCR fragments are required for NGS, comprising respectively SEQ ID NO:1 to SEQ ID NO:23), a reverse primer on the 3′ part of the IGHJ genes (comprising SEQ ID NO:30),
   from cDNA: forward primers located on the L1 part of the IGHV genes (comprising SEQ ID NO:24 to SEQ ID NO:29, and optionally SEQ ID NO: 133), reverse primers on the 5′ part of the IGHC genes (comprising respectively SEQ ID NO:31 to SEQ ID NO:33, and optionally SEQ ID NO: 134);

The NGS primers further contain in 5′ of the above-mentioned sequences adapter sequences useful for NGS sequencing and multiplexing.
(ii) For Sanger sequencing
   from gDNA: forward primers located on the L1 part of the IGHV genes (comprising respectively SEQ ID NO:24 to SEQ ID NO:29, and optionally SEQ ID NO: 133), a reverse primer on the 3′ part of the IGHJ genes (comprising SEQ ID NO:30),
   from cDNA: same forward IGHV-L1 primers (comprising respectively SEQ ID NO:24 to SEQ ID NO:29, and optionally SEQ ID NO: 133), reverse primers on the 5′ part of the IGHC genes (comprising respectively SEQ ID NO:31 to SEQ ID NO:33, and optionally SEQ ID NO: 134).

In addition, while gDNA is the main type of DNA analyzed for determination of CLL mutational status, in a small proportion of cases it may lead to no result, in particular when somatic hypermutations (SHM) are present in the L2 IGHV or in the IGHJ genes. Therefore, a methodology resulting in a very high rate of success in determination of the IGHV mutational status in CLL requires the possibility of further analyzing cDNA of the same patient. The inventors therefore defined a methodology based on division of the CLL patient's biological sample into 2 parts, gDNA extracted from the first part being first analyzed using a first set of primers and, only if necessary, cDNA extracted from the second part is further analyzed using a second set of primers, wherein the first and second sets of primers are slightly different depending whether NGS or Sanger sequencing is used. In both cases, the complete kit containing both the first and second sets of primers for analysis of both gDNA and cDNA contains forward primers on the L1 part of the IGHV genes comprising respectively SEQ ID NO:24 to SEQ ID NO:29, and optionally SEQ ID NO: 133, and reverse primers on the 5′ part of the IGHC genes comprising respectively SEQ ID NO:31 to SEQ ID NO:33, and optionally SEQ ID NO: 134. In addition, while the first and second sets of primers for analysis of gDNA and cDNA, respectively, may be commercialized separately; both are needed (although not in the same amount, as gDNA will generally be analyzed more often than cDNA) as they are complementary in order to ensure a success rate as high as disclosed herein. Finally, kit versions need to be adapted to the sequencing methodology, e.g. NGS or Sanger, as the former requires specific primer modifications (adapters).

Furthermore, as a control to their method for determining the mutational status of a CLL patient, the inventors have also designed an internal control, comprising a mixture of plasmids encoding clonal productive rearranged heavy chain immunoglobulin genes using each of the distinct functional IGHV genes (comprising the sequences SEQ ID NO:76 to SEQ ID NO:122). This internal control is useful to ensure that all primer combinations work appropriately and to evaluate their amplification efficiency.

In a first aspect, the present invention thus relates to a kit for determining the mutational status of a patient suffering from CLL from gDNA or cDNA extracted from a biological sample of said patient, said kit comprising:
a) forward primers comprising respectively the sequences SEQ ID NO:1 to SEQ ID NO:23 and a reverse primer comprising SEQ ID NO:30;
b) forward primers comprising respectively the sequences SEQ ID NO:24 to SEQ ID NO:29, and optionally SEQ ID NO: 133, and a reverse primer comprising SEQ ID NO:30;
c) forward primers comprising respectively the sequences SEQ ID NO:24 to SEQ ID NO:29, and optionally SEQ ID NO: 133, and reverse primers comprising respectively the sequences SEQ ID NO:31 to SEQ ID NO:33, and optionally SEQ ID NO: 134; and/or
d) nucleic acid molecules comprising the sequences SEQ ID NO:76 to SEQ ID NO:122.

The present invention also relates to a method for determining the mutational status of a patient suffering from B-cell chronic lymphocytic leukemia (CLL) from a biological sample of said CLL patient, comprising the steps of:
a) obtaining genomic DNA (gDNA) and/or complementary DNA (cDNA) from the biological sample,
b) amplifying rearranged immunoglobulin heavy chain genes from gDNA and/or cDNA by multiplex polymerase chain reaction (PCR) using primers from the kit according to the invention;
c) sequencing amplified rearranged heavy chain immunoglobulin genes using either Sanger or NGS sequencing depending on the composition of the kit and identifying a clonal productive rearranged heavy chain immunoglobulin gene,
d) aligning the identified clonal productive rearranged heavy chain immunoglobulin gene to germline immunoglobulin IGHV, IGHD and IGHJ genes, determining the percentage of identity between the IGHV gene of the identified clonal productive rearranged heavy chain immunoglobulin gene and its closest germline immunoglobulin IGHV gene, and
e) determining the mutational status of the CLL patient, wherein the mutational status is:
   unmutated if the percentage of identity between the IGHV gene of the identified clonal productive rearranged heavy chain immunoglobulin gene and its closest germline immunoglobulin IGHV gene is equal to or higher than 98% (e.g. 2% or less mutations), or
   mutated if the percentage of identity between the IGHV gene of the identified clonal productive rearranged heavy chain immunoglobulin gene and its closest germline immunoglobulin IGHV gene is below 98% (e.g. more than 2% mutations)

### DESCRIPTION OF THE FIGURES

FIG. 1. Anatomy of a rearranged IGH gene. Structure of the BCR is presented, as well as the organization of IGH V, D and J germline genes, and the structure of a rearranged IGH gene (in this rearranged IGH gene, mutations compared to the closest germline genes are represented by *). Note that only mutations within the IGHV gene are taken into account for defining the mutational status in clinical use

FIG. 2. IGHV mutational status provided by IMGT/V-QUEST. After entering a specific rearranged IGH sequence into IMGT/V-QUEST software, the software provides the identity of the closest IGH V, D and J genes of the V part of the rearranged IGH sequence and determines the % of identity of the IGHV gene to the closest germline IGHV gene. The mutational status (U-CLL or M-CLL) is then determined depending on the % of identity of the IGHV gene to the closest germline IGHV gene: the analyzed CLL is an unmutated CLL (U-CLL) if the IGHV % of identity is higher or equal to 98%, and a mutated CLL (M-CLL) if the IGHV % of identity is lower than 98%.

FIG. 3. IGHV mutational status assessment (NGS). A scheme of an optimized method for determining the mutational status of a CLL patient using NGS sequencing is presented. The patient sample is divided into 2 parts. gDNA is extracted from the first part, the second part may be used for RNA extraction and cDNA conversion or preferably conserved for possible future use as a dry cell pellet or as a cell lysate after extraction with a solution comprising a chaotropic agent. IGH-VDJ rearrangements are amplified from extracted gDNA by PCR using a first set of primers comprising IGH-L2 forward primers and IGH-J reverse primers. The amplified IGH-VDJ rearrangements are then sequenced using NGS, a clonal CLL productive IGH-VDJ rearrangement is identified using ARRest/Interrogate or Vidjil or any other appropriate software and the identified clonal CLL productive IGH-VDJ rearrangement is then analyzed using IMGT/V QUEST software. If no clonal CLL productive IGH-VDJ rearrangement is identified after NGS sequencing, then IGH-VDJ rearrangements are amplified from cDNA obtained from the second part of the biological sample using a second set of primers comprising IGH-L1 forward primers and IGH-C reverse primers. The amplified IGH-VDJ rearrangements are then sequenced using NGS, a clonal CLL productive IGH-VDJ rearrangement is identified using ARRest/Interrogate or Vidjil or any other appropriate software and the identified clonal CLL productive IGH-VDJ rearrangement is then analyzed using IMGT/V QUEST software.

FIG. 4. IGHV mutational status assessment (Sanger). A scheme of an optimized method for determining the mutational status of a CLL patient using Sanger sequencing is presented. The patient sample is divided into 2 parts. gDNA is extracted from the first part, the second part may be used for RNA extraction and cDNA conversion or preferably conserved for possible future use as a dry cell pellet or as a cell lysate after extraction with a solution comprising a chaotropic agent. IGH-VDJ rearrangements are amplified from extracted gDNA by PCR using a first set of primers comprising IGH-L1 forward primers and IGH-J reverse primers. The amplified IGH-VDJ rearrangements are then sequenced by Sanger method, and the sequences are then analyzed using IMGT/V QUEST software for identification of the clonal CLL productive IGH-VDJ rearrangement. If no clonal CLL productive IGH-VDJ rearrangement is identified after Sanger sequencing, then IGH-VDJ rearrangements are amplified from cDNA obtained from the second part of the biological sample using a second set of primers comprising IGH-L1 forward primers and IGH-C reverse primers. The amplified IGH-VDJ rearrangements are then sequenced using Sanger sequencing, and then analyzed using IMGT/V QUEST software to identify the clonal CLL productive IGH-VDJ rearrangement

FIG. 5. Plasmid mix control. A unique pool of 47 plasmids at equimolar concentrations has been obtained after cloning CLL clonal productive IGH-VDJ rearrangements of 47 distinct CLL patients. Each of the rearrangements has a distinct IGHV gene corresponding to all but one functional human IGH genes (the one not included being very rarely used in CLL). The 47 distinct plasmids have then been mixed at equimolar concentration to provide an internal control for the method for determining the IGHV mutational status of CLL patients according to the invention.

FIG. 6. Impact of primer and PCR conditions optimization for 2 CLL cases with biallelic IGH-VDJ rearrangements. Illustration of the clonotype sizes and frequencies after NGS gDNA-based assay and data analysis using the Vidjil software. These two CLL cases display biallelic IGH-VDJ rearrangements with clear unbalanced proportions. The clonotype sizes are indicated in grey characters above each case.

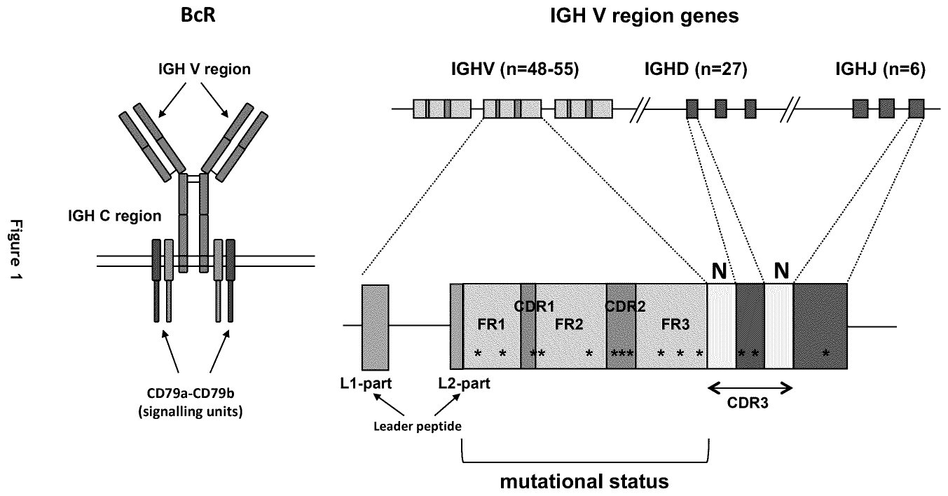

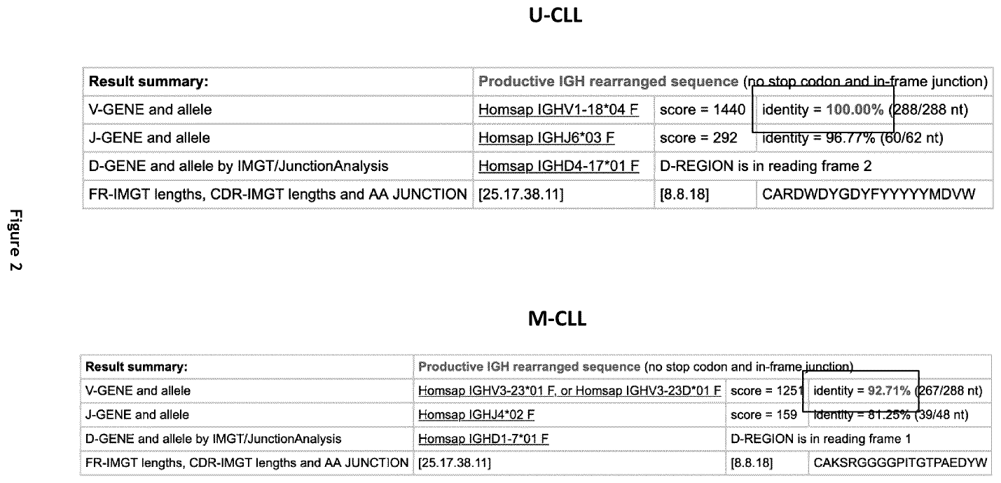

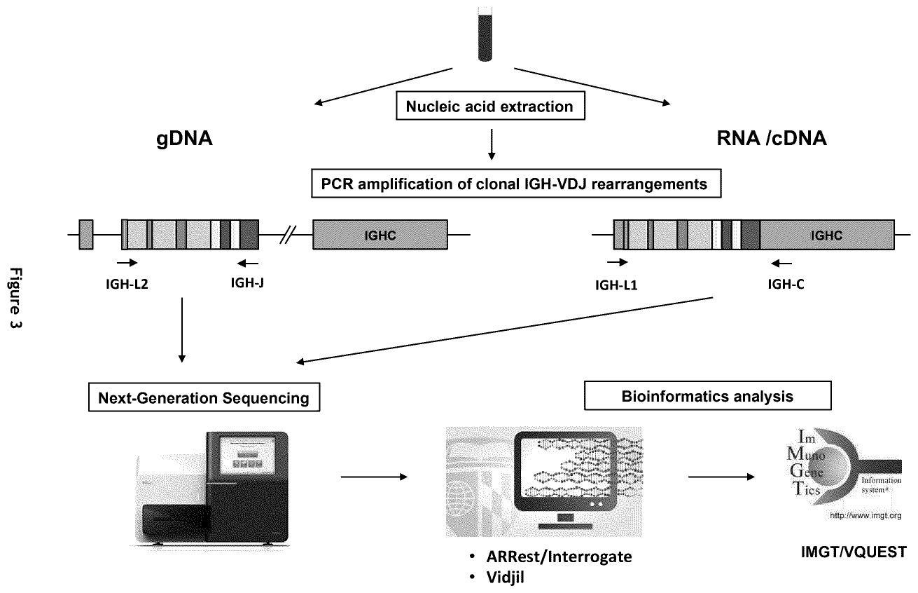

Case A: The productive (P) rearrangement using the IGHV1-69 gene on one allele was initially detected at a lower frequency than the unproductive (UP) rearrangement using the IGHV3-71 gene on the other allele allele (18.9% vs 64.3%). After protocol optimization the clonotypes frequencies became very similar (38.0% vs 39.5%).

Case B: The productive (P) rearrangement using the IGHV1-46 gene on one allele was initially detected at a much lower frequency than the unproductive (UP) rearrangement using the IGHV3-15 gene on the other allele (3.5% vs 72.2%). After protocol optimization the clonotypes frequencies became similar (32.9% vs 40.0%).

FIG. 7. Protocol optimization: evaluation of Mix #1 (top) and Mix #12 (bottom) on the 47 plasmid mix. Illustration of the clonotype frequencies of each IGH-VDJ rearrangement present in the 47 plasmid mix after NGS gDNA-based assay and data analysis using the Vidjil software. The plasmids and corresponding IGHV genes have been ranked according to their clonotype frequency. In absence of amplification bias, each IGH-VDJ rearrangement should be detected at a frequency close to 2%. The shaded zone indicates the 2%±1% range.

FIG. 8. Primers and PCR conditions changes during protocol optimization. The sequences of primers of Mix #1 (left) and Mix #12 (right) are indicated, as well as the concentration in Mg²⁺ ions and extension temperature used with each mix. Changes are highlighted (cases in grey in Mix 12 correspond to modifications compared to Mix 1, modified sequences and ratios are in bold). * These initial primers were either deleted (IGHL2-3.2*) or changed in their numbering (IGHL2-3.3* to IGHL2-3.6* becoming IGHL2-3.2 to IGHL2-3.5).

FIG. 9. NGS gDNA-based protocol (Validation cohort 2). The proportion of 564 gDNA samples of CLL patients analyzed by NGS for which IGHV mutational status was determined, and the proportions of gDNA samples analyzed by NGS for which mutational status could not be determined due to IGHV-L2 mutations, IGHJ mutations or undetermined reasons are indicated.

FIG. 10. Validation cohort 3: clonotypes frequencies. The clonotypes frequencies of main productive (P) rearrangements (top) and all productive (P) and unproductive (UP) rearrangements (bottom) obtained for 23 CLL cases by the reference laboratory and 4 distinct validation laboratories are presented. Note that one validation laboratory (#3) failed to detect productive P05 and P06 rearrangements.

FIG. 11. Sanger gDNA-based protocol (Validation cohort 4). The proportion of 647 gDNA samples of CLL patients analyzed by Sanger sequencing for which IGHV mutational status was determined, and the proportions of gDNA samples analyzed by NGS for which mutational status could not be determined due to IGHV-L2 mutations, IGHJ mutations or undetermined reasons are indicated.

FIG. 12. cDNA rescue for gDNA failures/suboptimal results. The results of IGHV mutational status assessment obtained using NGS sequencing on cDNA samples from 59 CLL problematic cases (41 cases for which no clonal productive rearrangement could be detected by gDNA NGS-based method and 18 additional cases for which results using the gDNA NGS-based method were uncertain due the detection of either a single minor clonal rearrangement, or an unbalance between a minor productive rearrangement and a major unproductive one, or more than 2 rearrangements) are presented. In all cases but one, a productive rearrangement could be sequenced based on cDNA sample and IGHV mutational status determined.

FIG. 13. Impact of optimization of IGHV3-64D gene detection by NGS from cDNA template. A CLL case having a clonal productive rearrangement bearing the IGHV3-64D gene (as determined from gDNA template) was analyzed at the cDNA level. Using the former version of the IGHL1 primers, the productive IGHV3-64D was very weakly amplified, appearing only as a minor clone on a polyclonal background (top panel). In contrast, with the optimized version of IGHL1 primers which includes a IGHV3-64D specific primer, the IGHV3-64D rearrangement appeared clearly as a dominant clonal one (bottom panel).

FIG. 14. Optimization of IGHC primers by inclusion of IGHC-alpha primer. A CLL case was analyzed first at the gDNA level but only an unproductive IGHV4-30-4—IGHJ4 rearrangement was obtained (left panel). A new attempt was made from cDNA, and again the same unproductive rearrangement (IGHV4-30-4—IGHC-gamma) was detected (middle panel). Upon addition of a IGHC-alpha primer in the IGHC primer mix, a productive IGHV3-30—IGHC-alpha rearrangement was obtained, allowing IGHV mutational status assessment of this case (right panel).

### DETAILED DESCRIPTION OF THE INVENTION

### Kits for Determining the Mutational Status of CLL Patients

The present invention first relates to a kit for determining the mutational status of a patient suffering from CLL from gDNA or cDNA extracted or obtained from a biological sample of said patient, said kit comprising:
a) forward primers comprising respectively the sequences SEQ ID NO:1 to SEQ ID NO:23 and a reverse primer comprising SEQ ID NO:30;
b) forward primers comprising respectively the sequences SEQ ID NO:24 to SEQ ID NO:29 and a reverse primer comprising SEQ ID NO:30;
c) forward primers comprising respectively the sequences SEQ ID NO:24 to SEQ ID NO:29 and reverse primers comprising respectively the sequences SEQ ID NO:31 to SEQ ID NO:33; and/or
d) plasmids comprising respectively the sequences SEQ ID NO:76 to SEQ ID NO:122.

In this kit:
the set of primers a) corresponds to amplification primers useful for determining the mutational status of a CLL patient from gDNA using NGS sequencing,
the set of primers b) corresponds to amplification primers useful for determining the mutational status of a CLL patient from gDNA using Sanger sequencing and may optionally further comprise a forward primer comprising the sequence SEQ ID NO: 133,
the set of primers c) corresponds to amplification primers useful for determining the mutational status of a CLL patient from cDNA using NGS or Sanger sequencing and may optionally further comprise a forward primer comprising the sequence SEQ ID NO: 133, a reverse primer comprising the sequence SEQ ID NO: 134, or both a forward primer comprising the sequence SEQ ID NO: 133 and a reverse primer comprising the sequence SEQ ID NO:134,
plasmids of d) corresponds to the internal control.

The sets of primers a) and c) may be commercialized together or separately, but both are necessary as they are complementary in order to use the high success rate methodology disclosed herein for NGS sequencing based on first analysis of gDNA, followed if necessary by secondary analysis of cDNA, disclosed herein.

Similarly, the sets of primers b) and c) may be commercialized together or separately, but both are necessary as they are complementary in order to use the high success rate methodology disclosed herein for Sanger sequencing based on first analysis of gDNA, followed, if necessary, by secondary analysis of cDNA, disclosed herein.

### Kits for Determination of CLL Mutational Status Using NGS Sequencing

A first type of kit is for determination of CLL mutational status using NGS sequencing.

Analysis of gDNA

As NGS is suitable only for nucleic acids of limited size, and the IGHV-L1 and IGHV-L2 regions, on the one hand, and the IGHJ and IGHC regions, on the other hand, are separated from one another by introns, forward primers for amplification of rearranged heavy chain immunoglobulin genes from gDNA before NGS sequencing have been designed in the IGHV-L2 region and a reverse primer has been designed in the 3′ part of the IGHJ region.

Therefore, in an embodiment directed to determination of CLL mutational status from gDNA using NGS sequencing, the kit according to the invention comprises forward primers comprising respectively the sequences SEQ ID NO:1 to SEQ ID NO:23 and a reverse primer comprising SEQ ID NO:30.

These primers contain the following target sequences:

TABLE 1
Target sequences of forward and reverse primers for amplification of
rearranged heavy chain immunoglobulin genes from gDNA before NGS
sequencing.

| Type of primer | Target | |
|---|---|---|
| (forward/reverse) | region | Target sequence (5′ to 3′) |
| Forward | IGHV-L2 | GTGTTCTCTCCACAGGAGCC (SEQ ID NO: 1) |
| Forward | IGHV-L2 | GTGTCTTCTCTACAGGTGCCCA (SEQ ID NO: 2) |
| Forward | IGHV-L2 | GTGTTCTCTCCACAGGTGCC (SEQ ID NO: 3) |
| Forward | IGHV-L2 | GTGTCCTCTCCACAGGTGCC (SEQ ID NO: 4) |
| Forward | IGHV-L2 | CTGTCCTCTCCACAGGCACC (SEQ ID NO: 5) |
| Forward | IGHV-L2 | GTGTCCCCTCCACAGATGC (SEQ ID NO: 6) |
| Forward | IGHV-L2 | GTGTCCTCTCCGCAGGTG (SEQ ID NO: 7) |
| Forward | IGHV-L2 | GTGTCCTCTCCACAGGTGTCCAGTCC (SEQ ID NO: 8) |
| Forward | IGHV-L2 | TTCTCTTCTCCACAGGCACC (SEQ ID NO: 9) |
| Forward | IGHV-L2 | CTTATGTCTTCTCCACAGGGGTC (SEQ ID NO: 10) |
| Forward | IGHV-L2 | CTTATGCTTTCTCCACAGGGGT (SEQ ID NO: 11) |
| Forward | IGHV-L2 | TGTGTTTGCAGGTGTCCAGTG (SEQ ID NO: 12) |
| Forward | IGHV-L2 | TCTGTTTGCAGGTGTCCAGTG (SEQ ID NO: 13) |
| Forward | IGHV-L2 | TTTGTTTGCAGGTGTCCAGTG (SEQ ID NO: 14) |
| Forward | IGHV-L2 | TGTGTTTGCAGGTGTCCAATG (SEQ ID NO: 15) |
| Forward | IGHV-L2 | CGTGTTTGCAGGTGTCCAGT (SEQ ID NO: 16) |
| Forward | IGHV-L2 | TTTGTTTGCAGATGTCCAGTG (SEQ ID NO:17) |
| Forward | IGHV-L2 | GTCTCTCTGTTCACAGGGGTCC (SEQ ID NO: 18) |
| Forward | IGHV-L2 | GTTTCTCTGTTCACAGGGGTCC (SEQ ID NO: 19) |
| Forward | IGHV-L2 | GTTTTTCTGTTCACAGGGGTCC (SEQ ID NO: 20) |
| Forward | IGHV-L2 | TCTCCCCCACAGGAGTCTGT (SEQ ID NO: 21) |
| Forward | IGHV-L2 | TCTTCCATACAGGAGTCTGTGC (SEQ ID NO: 22) |
| Forward | IGHV-L2 | TGTCTCCAGGTGTCCTGTCAC (SEQ ID NO: 23) |
| Reverse | IGHJ (3′ part) | CTTACCTGAGGAGACGGTGACC (SEQ ID NO: 30) |

Forward primers comprising respectively the sequences SEQ ID NO:1 to SEQ ID NO:23 are preferably mixed in a single solution.

When all forward primers comprising respectively the sequences SEQ ID NO:1 to SEQ ID NO:23 are mixed in a single solution, all forward primers may be mixed at equimolar or distinct ratios.

Based on the amplifying efficiency of primers comprising respectively the sequences SEQ ID NO:1 to SEQ ID NO:23 tested by the inventors, forward primers comprising respectively the sequences SEQ ID NO: 1 to SEQ ID NO:23 are however preferably mixed not at equimolar ratio but at the following ratios:

TABLE 2
Appropriate ratios of forward primers in a mixture of all forward
primers for amplification of gDNA before NGS sequencing. The
molar amount of the forward primer of sequence SEQ ID NO:
1 has been arbitrarily set to 1, and the ratio of the molar
amount of each forward primer to the molar amount of the forward
primer of sequence SEQ ID NO: 1 is indicated.

| Forward primer target | |
|---|---|
| sequence identifier | Ratio |
| SEQ ID NO: 1 | 1 |
| SEQ ID NO: 2 | 0.75 to 1.25 (preferably 1) |
| SEQ ID NO: 3 | 0.75 to 1.25 (preferably 1) |
| SEQ ID NO: 4 | 0.75 to 1.25 (preferably 1) |
| SEQ ID NO: 5 | 0.75 to 1.25 (preferably 1) |
| SEQ ID NO: 6 | 0.75 to 1.25 (preferably 1) |
| SEQ ID NO: 7 | 1.75 to 2.25 (preferably 2) |
| SEQ ID NO: 8 | 1.75 to 2.25 (preferably 2) |
| SEQ ID NO: 9 | 0.75 to 1.25 (preferably 1) |
| SEQ ID NO: 10 | 1.00 to 1.50 (preferably 1.25) |
| SEQ ID NO: 11 | 1.00 to 1.50 (preferably 1.25) |
| SEQ ID NO: 12 | 0.75 to 1.25 (preferably 1) |
| SEQ ID NO: 13 | 0.75 to 1.25 (preferably 1) |
| SEQ ID NO: 14 | 0.75 to 1.25 (preferably 1) |
| SEQ ID NO: 15 | 0.75 to 1.25 (preferably 1) |
| SEQ ID NO: 16 | 0.75 to 1.25 (preferably 1) |
| SEQ ID NO: 17 | 0.75 to 1.25 (preferably 1) |
| SEQ ID NO: 18 | 0.50 to 1.00 (preferably 0.75) |
| SEQ ID NO: 19 | 0.50 to 1.00 (preferably 0.75) |
| SEQ ID NO: 20 | 0.50 to 1.00 (preferably 0.75) |
| SEQ ID NO: 21 | 2.25 to 2.75 (preferably 2.5) |
| SEQ ID NO: 22 | 2.25 to 2.75 (preferably 2.5) |
| SEQ ID NO: 23 | 2.25 to 2.75 (preferably 2.5) |

In the PCR reaction mix, the ratio of the forward primer mix comprising SEQ ID NO:1 to SEQ ID NO:23 to the reverse primer comprising SEQ ID NO:30 is preferably between 8:1 and 4:1, more preferably between 7:1 and 5:1, such as 6:1.

Analysis of cDNA

When CLL mutational status is determined from cDNA obtained from RNA extracted from a CLL patient biological sample, the constraint of the presence of introns between the IGHV-L1 and IGHV-L2 regions, on the one hand, and the IGHJ and IGHC regions, on the other hand, are no more present.

As a result, forward primers for amplification of rearranged heavy chain immunoglobulin genes from cDNA before NGS sequencing have been designed in the IGHV-L1 region and three reverse primers have been designed in the 5′ part of the IGHC region. The 3 IGHC primers target simultaneously different classes of constant regions including M-type, D-type and G-type, e.g. all those possibly expressed by CLL cells.

Therefore, in another embodiment directed to determination of CLL mutational status from cDNA using NGS sequencing, the kit according to the invention comprises forward primers comprising respectively the sequences SEQ ID NO:24 to SEQ ID NO:29, and reverse primers comprising respectively the sequences SEQ ID NO:31 to SEQ ID NO:33. This kit may further comprise a forward primer comprising the sequence SEQ ID NO: 133, a reverse primer comprising the sequence SEQ ID NO:134 or both a forward primer comprising the sequence SEQ ID NO:133 and a reverse primer comprising the sequence SEQ ID NO:134, as these additional primers have been found to permit the detection of rare CLL rearrangements (see Example 3 below).

These primers contain the following target sequences:

TABLE 3
Target sequences of forward and reverse primers for amplification of
rearranged heavy chain immunoglobulin genes from cDNA before NGS
sequencing. B = C or G or T; K = G or T; M = A or C; R = A or G; S = C or
G; W = A or T; Y = C or T.

| Type of primer | Target | |
|---|---|---|
| (forward/reverse) | region | Target sequence (5′ to 3′) |
| Forward | IGHV-L1 | CTCACCATGGACTGSAYYTGGAG (SEQ ID NO: 24) |
| Forward | IGHV-L1 | ATGGACAYACTTTGYTMCACRCTCC (SEQ ID NO: 25) |
| Forward | IGHV-L1 | ATGGARTTKGGGCTKWGCTGGGTTT (SEQ ID NO: 26) |
| Forward | IGHV-L1 | CTGTGGTTCTTYCTBCTSCTGGTGG (SEQ ID NO: 27) |
| Forward | IGHV-L1 | CCTCCTCCTRGCTRTTCTCCAAG (SEQ ID NO: 28) |
| Forward | IGHV-L1 | CTGTCTCCTTCCTCATCTTCCTGCC (SEQ ID NO: 29) |
| Forward | IGHV-L1 | ATGGAGTTCTGGCTGAGCTGGGTTC (SEQ ID NO: 133) |
| Reverse | IGHC-mu | GGTTGGGGCGGATGCACT (SEQ ID NO: 31) |
| | (5′ part) | |
| Reverse | IGHC-delta | GGGAACACATCCGGAGCCTTG (SEQ ID NO: 32) |
| | (5′ part) | |
| Reverse | IGHC-gamma | CGATGGGCCCTTGGTGGA (SEQ ID NO: 33) |
| | (5′ part) | |
| Reverse | IGHC-alpha | CTTGGGGCTGGTCGGGGAT (SEQ ID NO: 134) |
| | (5′ part) | |

Forward primers comprising respectively the sequences SEQ ID NO:24 to SEQ ID NO:29 (and optionally SEQ ID NO: 133) are preferably mixed in a single solution. In this case, all forward primers may be mixed at equimolar or distinct ratios.

Based on the amplifying efficiency of primers comprising respectively the sequences SEQ ID NO:24 to SEQ ID NO:29 tested by the inventors, forward primers comprising respectively the sequences SEQ ID NO:24 to SEQ ID NO:29 are preferably mixed at the following ratios:

TABLE 4A
Appropriate ratios of forward primers in a mixture of all forward
primers (SEQ ID NO: 24 to SEQ ID NO: 29) for amplification of cDNA
before NGS sequencing. The molar amount of the forward primer of
sequence SEQ ID NO: 24 has been arbitrarily set to 1, and the ratio
of the molar amount of each forward primer to the molar amount of
the forward primer of sequence SEQ ID NO: 24 is indicated.

| Forward primer target | |
|---|---|
| sequence identifier | Ratio |
| SEQ ID NO: 24 | 1 |
| SEQ ID NO: 25 | 0.75 to 1.25 (preferably 1) |
| SEQ ID NO: 26 | 0.75 to 1.25 (preferably 1) |
| SEQ ID NO: 27 | 0.75 to 1.25 (preferably 1) |
| SEQ ID NO: 28 | 0.75 to 1.25 (preferably 1) |
| SEQ ID NO: 29 | 0.25 to 0.75 (preferably 0.5) |

When forward primers further comprise a forward primer comprising the sequence SEQ ID NO: 133, forward primers comprising respectively the sequences SEQ ID NO:24 to SEQ ID NO:29 and SEQ ID NO: 133 are preferably mixed at the following ratios:

TABLE 4B
Appropriate ratios of forward primers in a mixture of all
forward primers (SEQ ID NO: 24 to SEQ ID NO: 29 and SEQ ID
NO: 133) for amplification of cDNA before NGS sequencing.
The molar amount of the forward primer of sequence SEQ ID
NO: 24 has been arbitrarily set to 1, and the ratio of the
molar amount of each forward primer to the molar amount of
the forward primer of sequence SEQ ID NO: 24 is indicated.

| Forward primer target | |
|---|---|
| sequence identifier | Ratio |
| SEQ ID NO: 24 | 1 |
| SEQ ID NO: 25 | 0.75 to 1.25 (preferably 1) |
| SEQ ID NO: 26 | 0.75 to 1.25 (preferably 1) |
| SEQ ID NO: 27 | 0.75 to 1.25 (preferably 1) |
| SEQ ID NO: 28 | 0.75 to 1.25 (preferably 1) |
| SEQ ID NO: 29 | 0.25 to 0.75 (preferably 0.5) |
| SEQ ID NO: 133 | 0.1 to 0.3 (preferably 0.2) |

Similarly, reverse primers comprising respectively the sequences SEQ ID NO:31 to SEQ ID NO:33 are preferably mixed in a single solution (in particular when the kit does not contain the reverse primer of sequence SEQ ID NO:34). In this case, all reverse primers may be mixed at equimolar or distinct ratios.

Based on the amplifying efficiency of primers comprising respectively the sequences SEQ ID NO:31 to SEQ ID NO:33 tested by the inventors, reverse primers comprising respectively the sequences SEQ ID NO:31 to SEQ ID NO:33 are preferably mixed at the following ratios:

TABLE 5A
Appropriate ratios of reverse primers in a mixture of all reverse
primers (SEQ ID NO: 31 to SEQ ID NO: 33) for amplification of cDNA
before NGS sequencing. The molar amount of the forward primer of
sequence SEQ ID NO: 31 has been arbitrarily set to 1, and the ratio
of the molar amount of each reverse primer to the molar amount of
the reverse primer of sequence SEQ ID NO: 31 is indicated.

| Reverse primer target | |
|---|---|
| sequence identifier | Ratio |
| SEQ ID NO: 31 | 1 |
| SEQ ID NO: 32 | 0.75 to 1.25 (preferably 1) |
| SEQ ID NO: 33 | 0.75 to 1.25 (preferably 1) |

Alternatively, reverse primers comprising respectively the sequences SEQ ID NO:31 to SEQ ID NO:33 may preferably be divided into one mixture comprising reverse primers comprising respectively the sequences SEQ ID NO:31 and SEQ ID NO:33, and a single primer comprising the sequence SEQ ID NO:32. In this case, reverse primers comprising respectively the sequences SEQ ID NO:31 and SEQ ID NO:33 are preferably mixed in a SEQ ID NO:33/SEQ ID NO:31 ratio of 0.75 to 1.25 (preferably 1).

When reverse primers further comprise reverse primers comprising the sequences SEQ ID NO: 134, two distinct mixtures are preferably used, one comprising SEQ ID NO:31 and SEQ ID NO:33, and the other comprising SEQ ID NO:32 and SEQ ID NO:134 may be used in two distinct amplification reactions. In this case, reverse primers comprising respectively the sequences SEQ ID NO:31 and SEQ ID NO:33, on the one hand, and reverse primers comprising respectively SEQ ID NO:32 and SEQ ID NO: 134, on the other hand, are preferably mixed at the following ratios:

TABLE 5B
Appropriate ratios of reverse primers in two mixtures of reverse
primers (SEQ ID NO: 31 and SEQ ID NO: 33, on the one hand,
and SEQ ID NO: 32 and SEQ ID NO: 134, on the other hand)
for amplification of cDNA before NGS sequencing. For the
first mixture, the molar amount of the reverse primer of
sequence SEQ ID NO: 31 has been arbitrarily set to 1, and
the ratio of the molar amount of the other reverse primer
to the molar amount of the reverse primer of sequence SEQ
ID NO: 31 is indicated. For the second mixture, the molar
amount of the reverse primer of sequence SEQ ID NO: 32 has
been arbitrarily set to 1, and the ratio of the molar amount
of the other reverse primer to the molar amount of the reverse
primer of sequence SEQ ID NO: 32 is indicated.

| | |
|---|---|
| Reverse primer target | |
| sequence identifier | Ratio |

First reverse primer mixture (IGHC-mu + IGHC-gamma)

| | |
|---|---|
| SEQ ID NO: 31 | 1 |
| SEQ ID NO: 33 | 0.75 to 1.25 (preferably 1) |

Second reverse primer mixture (IGHC-delta + IGHC-alpha)

| | |
|---|---|
| SEQ ID NO: 32 | 1 |
| SEQ ID NO: 134 | 0.25 to 0.75 (preferably 0.5) |

In the PCR reaction mix, the ratio of the forward primer mix comprising respectively the sequences SEQ ID NO:24 to SEQ ID NO:29 to the reverse primer mix comprising respectively the sequences SEQ ID NO:31 to SEQ ID NO:33 is preferably between 4:2 to 1:1, more preferably between 7:4 and 5:4, such as 3:2.

Analysis of gDNA and cDNA

In some cases, it may be necessary to analyze both gDNA and cDNA obtained from a CLL patient in order to be able to determine its CLL mutational status.

This will be the case when a clonal productive IGVH rearrangement is not sequenced when analyzing first gDNA (most of the time) or cDNA (rare). Absence of sequencing of a clonal productive IGVH rearrangement when analyzing gDNA may happen when somatic hypermutations (SHM) are present in the L2 IGHV region and/or in the IGHJ region targeted by the forward and reverse primers, respectively.

Therefore, in another embodiment directed to determination of CLL mutational status from gDNA and cDNA using NGS sequencing, the kit according to the invention may comprise both:
a) forward primers comprising respectively the sequences SEQ ID NO:1 to SEQ ID NO:23,
b) a reverse primer comprising SEQ ID NO:30,
c) forward primers comprising respectively the sequences SEQ ID NO:24 to SEQ ID NO:29 (and optionally SEQ ID NO: 133), and
d) reverse primers comprising respectively the sequences SEQ ID NO:31 to SEQ ID NO:33 (and optionally SEQ ID NO: 134).
a) and b) are intended for analysis of gDNA, while c) and d) are intended for analysis of cDNA.

Preferably, in such a kit:
Forward primers comprising respectively the sequences SEQ ID NO:1 to SEQ ID NO:23 are mixed in a single solution;
Forward primers comprising respectively the sequences SEQ ID NO:24 to SEQ ID NO:29 (and optionally SEQ ID NO: 133) are mixed in a single solution;
Reverse primers comprising respectively the sequences SEQ ID NO:31 to SEQ ID NO:33 are mixed in a single solution;
Reverse primers comprising respectively the sequences SEQ ID NO:31 and SEQ ID NO:33 are mixed in a first single solution and the reverse primer comprising (or preferably consisting of) the sequence SEQ ID NO:32 is in another second solution;
Forward primers comprising respectively the sequences SEQ ID NO:24 to SEQ ID NO:29 (and optionally SEQ ID NO: 133) and reverse primers comprising respectively the sequences SEQ ID NO:31 and SEQ ID NO:33 are mixed in a first single solution and forward primers comprising respectively the sequences SEQ ID NO:24 to SEQ ID NO:29 (and optionally SEQ ID NO: 133) and the reverse primer comprising the sequence SEQ ID NO:32 are mixed in a second single solution;
When the kit further comprises the reverse primer comprising the sequence SEQ ID NO: 134, then the reverse primers comprising respectively the sequences SEQ ID NO:31 and SEQ ID NO:33 are mixed in a first single solution, and the reverse primers comprising respectively the sequences SEQ ID NO:32 and SEQ ID NO:134 are mixed in a second single solution.

When forward or reverse primers are mixed in a single solution, they are preferably mixed in the ratios described above for kits intended for gDNA analysis or cDNA analysis by NGS sequencing (see notably Tables 2, and 4A, 4B, 5A and 5B above). In the PCR reaction mix, the ratio of forward primers mix to reverse primer or reverse primer mix is preferably as described above for kits intended for gDNA analysis or cDNA analysis by NGS sequencing.

Such a kit is useful for performing the high success rate methodology developed by the inventors based on primary gDNA analysis, followed if necessary by secondary cDNA analysis. However, for practical purposes, primers intended for analysis of gDNA and cDNA using NGS may be provided in separated kits, as described above. Indeed, while the amounts of a) and b), on the one hand, and c) and d), on the other hand, may be selected based on the probability that the first analysis (generally gDNA) does not lead to the sequencing of a clonal productive IGVH rearrangement (for instance, primers for gDNA analysis and primers for cDNA analysis may be provided in a 8/1 to 10/1 ratio), it may be simpler for users to order kits intended for gDNA analysis and kits intended for cDNA analysis separately.

### Adapter Sequences

NGS sequencing requires the use of adapter sequences in 5′ and 3′ of each DNA strand, as defined above.

As a result, in addition to the target sequences presented above, the primers present in any kit directed to determination of the CLL mutational status by NGS sequencing disclosed above (gDNA, cDNA or gDNA+cDNA) may contain appropriate adapter sequences.

In this case, each primer will preferably consist, from 5′ to 3′, of an adapter sequence fused to the target sequence.

For instance, a kit for determination of the CLL mutational status by NGS sequencing from gDNA will preferably comprise:
forward primers consisting, from 5′ to 3′, of a first adapter sequence fused to one of the sequences SEQ ID NO:1 to SEQ ID NO:23, and
a reverse primer consisting, from 5′ to 3′, of a second adapter sequence fused to SEQ ID NO:30.

The first adapter sequence and the second adapter sequence included in the forward and reverse primers, respectively, are referred to as an adapter pair. Preferably the two adapter sequences of an adapter pair are distinct.

Such primers may be mixed as disclosed above for any kit directed to determination of the CLL mutational status by NGS sequencing (see in particular above-disclosed mixtures and preferred ratios).

The adapter sequences present in amplification primers intended for NGS sequencing may be selected from any adapter sequence disclosed as suitable for NGS sequencing by the manufacturer.

For instance, for sequencing on an Illumina MiSeq platform, suitable adapter sequences for forward primers have the following structure: 5′-Forward flow cell binding adapter-Index i5-Forward sequencing primer site-3′, wherein:
the Forward flow cell binding adapter is of sequence AATGATACGGCGACCACCGAGATCTACAC (SEQ ID NO:34),
the Forward sequencing primer site is of sequence ACACTCTTTCCCTACACGACGCTCTTCCGATCT (SEQ ID NO:35), and
the Index i5 is selected from one of the sequences SEQ ID NO: 36 to 53 of Table 6 below:

| Index i5 | |
|---|---|
| name | Sequence |
| D501 | TATAGCCT (SEQ ID NO: 36) |
| D502 | ATAGAGGC (SEQ ID NO: 37) |
| D503 | CCTATCCT (SEQ ID NO: 38) |
| D504 | GGCTCTGA (SEQ ID NO: 39) |
| D505 | AGGCGAAG (SEQ ID NO: 40) |
| D506 | TAATCTTA (SEQ ID NO: 41) |
| D507 | CAGGACGT (SEQ ID NO: 42) |
| D508 | GTACTGAC (SEQ ID NO: 43) |
| D509 | TTCGGATG (SEQ ID NO: 44) |
| D510 | ACTCATAA (SEQ ID NO: 45) |
| D511 | GCGCCTCT (SEQ ID NO: 46) |
| D512 | CGCGGCTA (SEQ ID NO: 47) |
| D513 | TTATTCGT (SEQ ID NO: 48) |
| D514 | CCTACGAA (SEQ ID NO: 49) |
| D515 | AGCAGATC (SEQ ID NO: 50) |
| D516 | GCGGAGCG (SEQ ID NO: 51) |
| D517 | TACTTACT (SEQ ID NO: 52) |
| D518 | AGGAAGTC (SEQ ID NO: 53) |

For sequencing on an Illumina MiSeq platform, suitable adapter sequences for forward primers have the following structure: 5′-Reverse flow cell binding adapter-Index i7—Reverse sequencing primer site-3′, wherein:
the Reverse flow cell binding adapter is of sequence CAAGCAGAAGACGGCATACGAGAT (SEQ ID NO:54),
the Reverse sequencing primer site is of sequence GTGACTGGAGTTCAGACGTGTGCTCTTCCGATCT (SEQ ID NO:55), and
the Index i7 is selected from one of the sequences of Table 7 below:

| Index i7 | |
|---|---|
| name | Sequence |
| D701 | ATTACTCG (SEQ ID NO: 56) |
| D702 | TCCGGAGA (SEQ ID NO: 57) |
| D703 | CGCTCATT (SEQ ID NO: 58) |
| D704 | GAGATTCC (SEQ ID NO: 59) |
| D705 | ATTCAGAA (SEQ ID NO: 60) |
| D706 | GAATTCGT (SEQ ID NO: 61) |
| D707 | CTGAAGCT (SEQ ID NO: 62) |
| D708 | TAATGCGC (SEQ ID NO: 63) |
| D709 | CGGCTATG (SEQ ID NO: 64) |
| D710 | TCCGCGAA (SEQ ID NO: 65) |
| D711 | TCTCGCGC (SEQ ID NO: 66) |
| D712 | AGCGATAG (SEQ ID NO: 67) |
| D713 | GAATAATC (SEQ ID NO: 68) |
| D714 | ATGCGGCT (SEQ ID NO: 69) |
| D715 | TTAATCAG (SEQ ID NO: 70) |
| D716 | ACTGCTTA (SEQ ID NO: 71) |
| D717 | CGTAGCTC (SEQ ID NO: 72) |
| D718 | GCCTCTCT (SEQ ID NO: 73) |
| D719 | GCCGTAGG (SEQ ID NO: 74) |
| D721 | CATCGAGG (SEQ ID NO: 75) |

In addition, for multiplexing purposes, distinct adapter pairs may be used when amplifying the rearranged heavy chain immunoglobulin genes of distinct patients. The amplified rearranged heavy chain immunoglobulin genes of several CLL patients may then be pooled and sequences by NGS simultaneously in a single reaction. According to the Illumina platform, with 18 available 5′ indexes and 20 available 3′ indexes, it is possible to sequence simultaneously up to 360 samples, each being identified by a unique dual index combination.

For instance, adapters using a 5′ index 501 of Table 6 above and a 3′ index 701 of Table 7 above may be used for a first patient, adapters using a 5′ index 501 of Table 6 above and a 3′ index 702 of Table 7 above may be used for a second patient, etc.

As explained above, each patient's sample is identified by using a distinct pair of 5′ (forward primers) and 3′ (reverse primers) indexes (specific index i5 for forward primers and specific index i7 for reverse primers). However, the same index i5 is used for all forward primers and the same index i7 is used for all reverse primers of a single patient.

A kit according to the invention may however comprise several primer mixtures varying only by the specific index (preferably index i5 for forward primers and index i7 for reverse primers) comprised in the forward or reverse primers sequences. In particular, a kit according to the invention may comprise:
For determination of CLL mutational status from gDNA using NGS sequencing:
   up to 18 distinct forward primer mixtures comprising forward primers consisting respectively, from 5′ to 3′, of a first adapter sequence (preferably of structure 5′-Forward flow cell binding adapter-Index i5-Forward sequencing primer site-3′, wherein the Forward flow cell binding adapter is of sequence SEQ ID NO:34, the Forward sequencing primer site is of sequence SEQ ID NO:35, and the Index i5 is one of the sequences SEQ ID NO:36 to 53 of Table 6 above) fused to one of the sequences SEQ ID NO:1 to SEQ ID NO:23, each forward primer mixture using a distinct Index i5, and
   up to 20 distinct reverse primers consisting, from 5′ to 3′, of a second adapter sequence (preferably of structure 5′-Reverse flow cell binding adapter-Index i7—Reverse sequencing primer site-3′, wherein the Reverse flow cell binding adapter is of sequence SEQ ID NO:54, the Reverse sequencing primer site is of sequence SEQ ID NO:55, and the Index i7 is one of the sequences SEQ ID NO:56 to 75 of Table 7 above) fused to SEQ ID NO:30
For determination of CLL mutational status from cDNA using NGS sequencing:
   up to 18 distinct forward primer mixtures comprising forward primers consisting respectively, from 5′ to 3′, of a first adapter sequence (preferably of structure 5′-Forward flow cell binding adapter-Index i5-Forward sequencing primer site-3′, wherein the Forward flow cell binding adapter is of sequence SEQ ID NO:34, the Forward sequencing primer site is of sequence SEQ ID NO:35, and the Index i5 is one of the sequences SEQ ID NO:36 to 53 of Table 6 above) fused to one of the sequences SEQ ID NO:24 to SEQ ID NO:29 (and optionally SEQ ID NO: 133), each forward primer mixture using a distinct Index i5, and
   up to 20 distinct reverse primer mixtures comprising reverse primers consisting, from 5′ to 3′, of a second adapter sequence (preferably of structure 5′-Reverse flow cell binding adapter-Index i7—Reverse sequencing primer site-3′, wherein the Reverse flow cell binding adapter is of sequence SEQ ID NO:54, the Reverse sequencing primer site is of sequence SEQ ID NO:55, and the Index i7 is one of the sequences SEQ ID NO:56 to 75 of Table 7 above) fused to one of the sequences SEQ ID NO:31 to SEQ ID NO:33 (and optionally SEQ ID NO:134).

By combining distinct pairs of forward primers mixture and reverse primer mixture differing by their index, up to 18×20=360 distinct patients' samples may be analyzed simultaneously. The numbers of primer mixtures differing by their index provided in a kit according to the invention may be varied depending on the user's need. For instance, kits may be provided with numbers of primer mixtures permitting simultaneous analysis of 6, 12, 18, 24, 36, 48, 60, 72, 84, or 96 distinct patients samples, by using;

TABLE 8
Possible combinations of numbers of distinct forward primer
mixtures and distinct reverse primer mixtures depending
on the total number of distinct patients' samples.

| | Examples of suitable (number of distinct |
|---|---|
| Number of distinct | forward primer mixtures) × (number of distinct |
| patients' samples | reverse primer mixtures) |
| 6 | 1 × 6, 2 × 3, 3 × 2 or 6 × 1 |
| 12 | 1 × 12, 2 × 6, 3 × 4, 4 × 3, 6 × 2, or 12 × 1 |
| 18 | 1 × 18, 2 × 9, 3 × 6, 6 × 3, 9 × 2, or 18 × 1 |
| 24 | 2 × 12, 3 × 8, 4 × 6, 6 × 4, 8 × 3, or 12 × 2 |
| 36 | 2 × 18, 3 × 12, 4 × 9, 6 × 6, 9 × 4, 12 × 3, |
| | or 18 × 2 |
| 48 | 3 × 16, 4 × 12, 6 × 8, 8 × 6, 12 × 4, or 16 × 3 |
| 60 | 4 × 15, 5 × 12, 6 × 10, 10 × 6, 12 × 5, or 15 × 4 |
| 72 | 4 × 18, 6 × 12, 12 × 6, or 18 × 4 |
| 84 | 7 × 12 or 12 × 7 |
| 96 | 8 × 12 or 12 × 8 |

(e.g. 1 forward primer mixture and 6 distinct reverse primer mixtures, 2 distinct forward primer mixtures and 3 distinct reverse primer mixtures, 3 distinct forward primer mixtures and 2 distinct reverse primer mixtures, or 6 distinct forward primer mixtures and 1 reverse primer mixture), 12 (e.g. 1 forward primer mixture and 12 distinct reverse primer mixtures, 2 distinct forward primer mixtures and 6 distinct reverse primer mixtures, 3 distinct forward primer mixtures and 4 distinct reverse primer mixtures, 4 distinct forward primer mixtures and 3 distinct reverse primer mixtures, 6 distinct forward primer mixtures and 2 distinct reverse primer mixtures, or 6 distinct forward primer mixtures and 1 reverse primer mixture), 18 (e.g. 2 distinct forward primer mixtures and 9 distinct reverse primer mixtures, 3 distinct forward primer mixtures and 6 distinct reverse primer mixtures, 6 distinct forward primer mixtures and 3 distinct reverse primer mixtures, or 6 distinct forward primer mixtures and 2 distinct reverse primer mixtures),

In such case, the kit according to the invention may contain several containers (e.g. as many containers as the number of CLL patients to be analyzed simultaneously), each container comprising a mixture of forward and reverse primers with the same target sequences but distinct adapter pairs sequences. Preferred mixtures may be any preferred mixtures disclosed above for any kit directed to determination of the CLL mutational status by NGS sequencing (see in particular above-disclosed mixtures and preferred ratios).

### Kits for Determination of CLL Mutational Status Using Sanger Sequencing

A second type of kit is for determination of CLL mutational status using Sanger sequencing (or any other sequencing technique allowing sequencing of nucleic acids of a size similar to Sanger).

Analysis of gDNA

While Sanger sequencing is suitable for nucleic acids of higher size than NGS, the intronic regions between the IGHJ and IGHC regions is too long for amplification of rearranged heavy chain immunoglobulin genes from gDNA before Sanger sequencing. Therefore, while it has been possible to design forward primers for amplification of rearranged heavy chain immunoglobulin genes from gDNA before Sanger sequencing in the IGHV-L1 region (less prone to be targeted by SHM than the IGHV-L2 region), the reverse primer has still been designed in the 3′ part of the IGHJ region.

Therefore, in an embodiment directed to determination of CLL mutational status from gDNA using Sanger sequencing, the kit according to the invention comprises forward primers comprising respectively the sequences SEQ ID NO:24 to SEQ ID NO:29 and a reverse primer comprising SEQ ID NO:30 (see Table 3 and Table 1 above, respectively, for definition of these sequences). This kit may further comprise a forward primer comprising the sequence SEQ ID NO:133 (see Example 3 below).

As no adapter sequences are necessary for Sanger sequencing, such a kit preferably comprises forward primers consisting respectively of the sequences SEQ ID NO:24 to SEQ ID NO:29 and a reverse primer consisting of SEQ ID NO:30.

Preferably, in such a kit:
i) Forward primers comprising (or preferably consisting of) respectively the sequences SEQ ID NO:24 to SEQ ID NO:29 (and optionally SEQ ID NO: 133) are mixed in a single solution; or
ii) Forward primers comprising (or preferably consisting of) respectively the sequences SEQ ID NO:24 to SEQ ID NO:29 (and optionally SEQ ID NO: 133) and reverse primer comprising (or preferably consisting of) SEQ ID NO:30 are mixed in a single solution.
   Embodiment ii) is preferred for Sanger sequencing.

A mixture of forward primers comprising (or preferably consisting of) respectively the sequences SEQ ID NO:24 to SEQ ID NO:29 (and optionally SEQ ID NO: 133) may use any appropriate ratios for the primers, including the preferred ratios disclosed in Table 4A or 4B above.

A mixture of forward primers comprising (or preferably consisting of) respectively the sequences SEQ ID NO:24 to SEQ ID NO:29 (and optionally SEQ ID NO: 133) and of reverse primer comprising (or preferably consisting of) SEQ ID NO:30 may use any appropriate ratios for the primers, including the preferred ratios disclosed in Table 4A or 4B above for SEQ ID NO:24 to SEQ ID NO:29 (and optionally SEQ ID NO: 133), and a ratio between 0.75 and 1.25 (preferably 1) for SEQ ID NO:30 (this ratio is expressed as the amount of SEQ ID NO:30 to the amount of SEQ ID NO:24 fixed to 1).

Analysis of cDNA

When CLL mutational status is determined from cDNA obtained from RNA extracted from a CLL patient biological sample, the constraint of the presence of a large intronic region between the IGHJ and IGHC regions is no more present, and forward primers for amplification of rearranged heavy chain immunoglobulin genes from cDNA before Sanger sequencing have been designed in the IGHV-L1 region and three reverse primers have been designed in the 5′ part of the IGHC region, similarly as for amplification of rearranged heavy chain immunoglobulin genes before NGS sequencing when starting from cDNA.

Therefore, in another embodiment directed to determination of CLL mutational status from cDNA using Sanger sequencing, the kit according to the invention comprises forward primers comprising respectively the sequences SEQ ID NO:24 to SEQ ID NO:29, and reverse primers comprising respectively the sequences SEQ ID NO:31 to SEQ ID NO:33. This kit may further comprise a forward primer comprising the sequence SEQ ID NO: 133, a reverse primer comprising the sequence SEQ ID NO:134 or both a forward primer comprising the sequence SEQ ID NO:133 and a reverse primer comprising the sequence SEQ ID NO:134, as these additional primers have been found to permit the detection of rare CLL rearrangements (see Example 3 below). These primers are disclosed in Table 3 above.

As no adapter sequences are necessary for Sanger sequencing, such a kit preferably comprises forward primers consisting respectively of the sequences SEQ ID NO:24 to SEQ ID NO:29 and reverse primers consisting respectively of the sequences SEQ ID NO:31 to SEQ ID NO:33. This kit may further comprise a forward primer consisting of the sequence SEQ ID NO:133, a reverse primer consisting of the sequence SEQ ID NO:134 or both a forward primer consisting of the sequence SEQ ID NO: 133 and a reverse primer consisting of the sequence SEQ ID NO:134.

Preferably, in such a kit:
i) Forward primers comprising (or preferably consisting of) respectively the sequences SEQ ID NO:24 to SEQ ID NO:29 (and optionally SEQ ID NO: 133) are mixed in a single solution;
ii) Reverse primers comprising (or preferably consisting of) respectively the sequences SEQ ID NO:31 to SEQ ID NO:33 are mixed in a single solution;
iii) Forward primers comprising (or preferably consisting of) respectively the sequences SEQ ID NO:24 to SEQ ID NO:29 (and optionally SEQ ID NO:133) and reverse primers comprising (or preferably consisting of) respectively the sequences SEQ ID NO:31 to SEQ ID NO:33 are mixed in a single solution;
iv) Reverse primers comprising (or preferably consisting of) respectively the sequences SEQ ID NO:31 and SEQ ID NO:33 are mixed in a first single solution and the reverse primer comprising (or preferably consisting of) the sequence SEQ ID NO:32 is in another second solution;
v) Forward primers comprising (or preferably consisting of) respectively the sequences SEQ ID NO:24 to SEQ ID NO:29 (and optionally SEQ ID NO: 133) and reverse primers comprising (or preferably consisting of) respectively the sequences SEQ ID NO:31 and SEQ ID NO:33 are mixed in a first single solution and forward primers comprising (or preferably consisting of) respectively the sequences SEQ ID NO:24 to SEQ ID NO:29 (and optionally SEQ ID NO: 133) and the reverse primer comprising (or preferably consisting of) the sequence SEQ ID NO:32 are mixed in a second single solution;
vi) When the kit further comprises the reverse primer comprising (or preferably consisting of) the sequence SEQ ID NO: 134, then the reverse primers comprising respectively the sequences SEQ ID NO:31 and SEQ ID NO:33 are mixed in a first single solution, and the reverse primers comprising respectively the sequences SEQ ID NO:32 and SEQ ID NO:134 are mixed in a second single solution;
vii) When the kit further comprises the reverse primer comprising (or preferably consisting of) the sequence SEQ ID NO: 134, then forward primers comprising (or preferably consisting of) respectively the sequences SEQ ID NO:24 to SEQ ID NO:29 (and optionally SEQ ID NO:133) and reverse primers comprising respectively the sequences SEQ ID NO:31 and SEQ ID NO:33 are mixed in a first single solution, and forward primers comprising (or preferably consisting of) respectively the sequences SEQ ID NO:24 to SEQ ID NO:29 (and optionally SEQ ID NO:133) and reverse primers comprising respectively the sequences SEQ ID NO:32 and SEQ ID NO:134 are mixed in a second single solution.

Embodiments iii) and v) are preferred for Sanger sequencing.

A mixture of forward primers comprising (or preferably consisting of) respectively the sequences SEQ ID NO:24 to SEQ ID NO:29 (and optionally SEQ ID NO:133) may use any appropriate ratios for the primers, including the preferred ratios disclosed in Tables 4A and 4B above.

A mixture of reverse primers comprising (or preferably consisting of) respectively the sequences SEQ ID NO:31 to SEQ ID NO:33 may use any appropriate ratios for the primers, including the preferred ratios disclosed in Table 5A above.

When the kit further comprises the reverse primer (or preferably consisting of) sequence SEQ ID NO: 134, then:
a mixture of forward primers comprising (or preferably consisting of) respectively the sequences SEQ ID NO:24 to SEQ ID NO:29 (and optionally SEQ ID NO:133) and of reverse primers comprising (or preferably consisting of) the sequences SEQ ID NO:31 and SEQ ID NO:33 may use any appropriate ratios for the primers, including the preferred ratios disclosed in Table 5B above; and
a mixture of forward primers comprising (or preferably consisting of) respectively the sequences SEQ ID NO:24 to SEQ ID NO:29 (and optionally SEQ ID NO:133) and of reverse primers comprising (or preferably consisting of) the sequences SEQ ID NO:32 and SEQ ID NO:134 may use any appropriate ratios for the primers, including the preferred ratios disclosed in Table 5B above.

A mixture of forward primers comprising (or preferably consisting of) respectively the sequences SEQ ID NO:24 to SEQ ID NO:29 (and optionally SEQ ID NO:133) and of reverse primers comprising (or preferably consisting of) respectively the sequences SEQ ID NO:31 to SEQ ID NO:33 may use any appropriate ratios for the primers. In particular, forward primers comprising (or preferably consisting of) respectively the sequences SEQ ID NO:24 to SEQ ID NO:29 (and optionally SEQ ID NO: 133) should preferably use the ratios disclosed in Tables 4A and 4B above, reverse primers comprising (or preferably consisting of) respectively the sequences SEQ ID NO:31 to SEQ ID NO:33 should preferably use the ratios disclosed in Table 5A above, and the ratio of the forward primer mix comprising (or preferably consisting of) respectively the sequences SEQ ID NO:24 to SEQ ID NO:29 (and optionally SEQ ID NO:133) to the reverse primer mix comprising (or preferably consisting of) respectively the sequences SEQ ID NO:31 to SEQ ID NO:33 (and optionally SEQ ID NO:134) is preferably between 4:2 to 1:1, more preferably between 7:4 and 5:4, such as 3:2.

When the kit further comprises the reverse primer (or preferably consisting of) sequence SEQ ID NO: 134, then a mixture of forward primers comprising (or preferably consisting of) respectively the sequences SEQ ID NO:24 to SEQ ID NO:29 (and optionally SEQ ID NO:133) and of reverse primers comprising (or preferably consisting of) respectively the sequences SEQ ID NO:31 and SEQ ID NO:33, on the one hand, and the sequences SEQ ID NO:32 and 134, on the other hand, may use any appropriate ratios for the primers. In particular, forward primers comprising (or preferably consisting of) respectively the sequences SEQ ID NO:24 to SEQ ID NO:29 (and optionally SEQ ID NO: 133) should preferably use the ratios disclosed in Tables 4A and 4B above, reverse primers comprising (or preferably consisting of) respectively the sequences SEQ ID NO:31 and SEQ ID NO:33, on the one hand, and the sequences SEQ ID NO:32 and 134, on the other hand, should preferably use the ratios disclosed in Table 5B above, and the ratio of the forward primer mix comprising (or preferably consisting of) respectively the sequences SEQ ID NO:24 to SEQ ID NO:29 (and optionally SEQ ID NO:133) to the reverse primer mix comprising (or preferably consisting of) respectively the sequences SEQ ID NO:31 and SEQ ID NO:33, on the one hand, and the sequences SEQ ID NO:32 and 134, on the other hand, is preferably between 4:2 to 1:1, more preferably between 7:4 and 5:4, such as 3:2.

Analysis of gDNA and cDNA

For Sanger sequencing too, it may be necessary in some cases (for instance when SHM are present in the IGHJ region targeted by the reverse primers, respectively) to analyze both gDNA and cDNA obtained from a CLL patient in order to be able to determine its CLL mutational status.

Therefore, in another embodiment directed to determination of CLL mutational status from gDNA and cDNA using NGS sequencing, the kit according to the invention may comprise both:
a) forward primers comprising respectively the sequences SEQ ID NO:24 to SEQ ID NO:29 (and optionally SEQ ID NO:133), preferably forward primers consisting respectively of the sequences SEQ ID NO:24 to SEQ ID NO:29 (and optionally SEQ ID NO:133);
b) a reverse primer comprising SEQ ID NO:30, preferably a reverse primer consisting of SEQ ID NO:30; and
c) reverse primers comprising respectively the sequences SEQ ID NO:31 to SEQ ID NO:33 (and optionally SEQ ID NO:134), preferably reverse primers consisting respectively of the sequences SEQ ID NO:31 to SEQ ID NO:33 (and optionally SEQ ID NO:134).
a) and b) are intended for analysis of gDNA, while a) and c) are intended for analysis of cDNA.

Preferably, in such a kit:
i) Forward primers comprising (or preferably consisting of) respectively the sequences SEQ ID NO:24 to SEQ ID NO:29 (and optionally SEQ ID NO: 133) are mixed in a single solution;
ii) Reverse primers comprising (or preferably consisting of) respectively the sequences SEQ ID NO:31 to SEQ ID NO:33 are mixed in a single solution;
iii) Forward primers comprising (or preferably consisting of) respectively the sequences SEQ ID NO:24 to SEQ ID NO:29 (and optionally SEQ ID NO:133) and reverse primer comprising (or preferably consisting of) SEQ ID NO:30 are mixed in a single solution;
iv) Forward primers comprising (or preferably consisting of) respectively the sequences SEQ ID NO:24 to SEQ ID NO:29 (and optionally SEQ ID NO:133) and reverse primers comprising (or preferably consisting of) respectively the sequences SEQ ID NO:31 to SEQ ID NO:33 are mixed in a single solution;
v) When the kit further comprises the reverse primer comprising the sequence SEQ ID NO: 134, then the reverse primers comprising (or preferably consisting of) respectively the sequences SEQ ID NO:31 and SEQ ID NO:33 are mixed in a first single solution, and the reverse primers comprising (or preferably consisting of) respectively the sequences SEQ ID NO:32 and SEQ ID NO: 134 are mixed in a second single solution; or
vi) When the kit further comprises the reverse primer comprising the sequence SEQ ID NO: 134, then forward primers comprising (or preferably consisting of) respectively the sequences SEQ ID NO:24 to SEQ ID NO:29 (and optionally SEQ ID NO:133) and the reverse primers comprising (or preferably consisting of) respectively the sequences SEQ ID NO:31 and SEQ ID NO:33 are mixed in a first single solution, and forward primers comprising (or preferably consisting of) respectively the sequences SEQ ID NO:24 to SEQ ID NO:29 (and optionally SEQ ID NO:133) and the reverse primers comprising (or preferably consisting of) respectively the sequences SEQ ID NO:32 and SEQ ID NO: 134 are mixed in a second single solution.

Embodiments iii), iv), v) and vi) are preferred for Sanger sequencing.

A mixture of forward primers comprising (or preferably consisting of) respectively the sequences SEQ ID NO:24 to SEQ ID NO:29 (and optionally SEQ ID NO:133) may use any appropriate ratios for the primers, including the preferred ratios disclosed in Tables 4A and 4B above.

A mixture of reverse primers comprising (or preferably consisting of) respectively the sequences SEQ ID NO:31 to SEQ ID NO:33 may use any appropriate ratios for the primers, including the preferred ratios disclosed in Table 5A above.

A first mixture of reverse primers comprising (or preferably consisting of) respectively the sequences SEQ ID NO:31 and SEQ ID NO:33 or a second mixture of reverse primers comprising (or preferably consisting of) respectively the sequences SEQ ID NO:32 and SEQ ID NO:134 may use any appropriate ratios for the primers, including the preferred ratios disclosed in Table 5B above.

A mixture of forward primers comprising (or preferably consisting of) respectively the sequences SEQ ID NO:24 to SEQ ID NO:29 (and optionally SEQ ID NO:133) and of reverse primer comprising (or preferably consisting of) SEQ ID NO:30 ay use any appropriate ratios for the primers, including the preferred ratios disclosed in Table 4A or 4B above for SEQ ID NO:24 to SEQ ID NO:29 (and optionally SEQ ID NO:133), and a ratio between 0.75 and 1.25 (preferably 1) for SEQ ID NO:30 (this ratio is expressed as the amount of SEQ ID NO:30 to the amount of SEQ ID NO:24 fixed to 1).

A mixture of forward primers comprising (or preferably consisting of) respectively the sequences SEQ ID NO:24 to SEQ ID NO:29 (and optionally SEQ ID NO:133) and of reverse primers comprising (or preferably consisting of) respectively the sequences SEQ ID NO:31 to SEQ ID NO:33 may use any appropriate ratios for the primers. In particular, forward primers comprising (or preferably consisting of) respectively the sequences SEQ ID NO:24 to SEQ ID NO:29 (and optionally SEQ ID NO: 133) should preferably use the ratios disclosed in Table 4A or 4B above, reverse primers comprising (or preferably consisting of) respectively the sequences SEQ ID NO:31 to SEQ ID NO:33 should preferably use the ratios disclosed in Table 5A above, and the ratio of the forward primer mix comprising (or preferably consisting of) respectively the sequences SEQ ID NO:24 to SEQ ID NO:29 (and optionally SEQ ID NO:133) to the reverse primer mix comprising (or preferably consisting of) respectively the sequences SEQ ID NO:31 to SEQ ID NO:33 is preferably between 4:2 to 1:1, more preferably between 7:4 and 5:4, such as 3:2.

When the kit further comprises the reverse primer comprising the sequence SEQ ID NO: 134, then a mixture of forward primers comprising (or preferably consisting of) respectively the sequences SEQ ID NO:24 to SEQ ID NO:29 (and optionally SEQ ID NO:133) and of reverse primers comprising (or preferably consisting of) respectively the sequences SEQ ID NO:31 and SEQ ID NO:33 and a mixture of forward primers comprising (or preferably consisting of) respectively the sequences SEQ ID NO:24 to SEQ ID NO:29 (and optionally SEQ ID NO:133) and of reverse primers comprising (or preferably consisting of) respectively the sequences SEQ ID NO:32 and SEQ ID NO:134 may use any appropriate ratios for the primers. In particular, forward primers comprising (or preferably consisting of) respectively the sequences SEQ ID NO:24 to SEQ ID NO:29 (and optionally SEQ ID NO:133) should preferably use the ratios disclosed in Table 4A or 4B above, reverse primers comprising (or preferably consisting of) respectively the sequences SEQ ID NO:31 and SEQ ID NO:33 and reverse primers comprising (or preferably consisting of) respectively the sequences SEQ ID NO:32 and SEQ ID NO:134 should preferably use the ratios disclosed in Table 5B above, and the ratio of the forward primer mix comprising (or preferably consisting of) respectively the sequences SEQ ID NO:24 to SEQ ID NO:29 (and optionally SEQ ID NO:133) to each reverse primer mix comprising (or preferably consisting of) respectively the sequences SEQ ID NO:31 and SEQ ID NO:33, on the one hand, or SEQ ID NO:32 and SEQ ID NO:134, on the other hand, is preferably between 4:2 to 1:1, more preferably between 7:4 and 5:4, such as 3:2.

### Internal Control

Any kit according to the invention as described above may further contain an internal control comprising nucleic acid molecules P01 to P47 comprising the sequences SEQ ID NO:76 to SEQ ID NO:122.

Each of SEQ ID NO:76 to SEQ ID NO:122 corresponds to a clonal productive rearranged heavy chain immunoglobulin gene from a CLL patient, each of SEQ ID NO:76 to SEQ ID NO:122 containing a distinct IGHV gene, all functional IGHV genes being represented in SEQ ID NO:76 to SEQ ID NO:122.

The IGHV gene and sequence of each of SEQ ID NO:76 to SEQ ID NO:122 are as follows:

TABLE 9
IGHV genes and sequences of the clonal productive rearranged heavy chain
immunoglobulin genes PO1 to P47 corresponding to SEQ ID NO: 76 to SEQ ID NO: 122.

| Internal | | |
|---|---|---|
| control | IGVH | |
| name | gene | Sequence |
| P01 | IGHV1- | Caggtgcagctggtgcagtctggggctgaggtgaagaagcctggggcctcagtgaaggtctcct |
| | 2 | gcaaggcttctggatacaccttcaccggctactatatgcactgggtgcgacaggcccctggacaa |
| | | gggcttgagtggatgggatggatcaaccctaacagtggtggcacaaactatgcacagaagtttca |
| | | gggcagggtcaccatgaccagggacacgtccatcagcacagcctacatggagctgagcaggct |
| | | gagatctgacgacacggccgtgtattactgtgcgaggtggcagtggctggtactaggatactttg |
| | | actactggggccagggaaccctggtcaccgtctcctcag (SEQ ID NO: 76) |
| P02 | IGHV1- | caggtccagcttgtgcagtctggggctgaggtgaagaagcctggggcctcagtgaaggtttcctg |
| | 3 | caaggcttctggatacaccttcactagctatgctatgcattgggtgcgccaggcccccggacaaa |
| | | ggcttgagtggatgggatggatcaacgctggcaatggtaacacaaaatattcacagaagttcca |
| | | gggcagagtcaccattaccagggacacatccgcgagcacagcctacatggagctgagcagcct |
| | | gagatctgaagacacggctgtgtattactgtgcgagaatgtatagtgggagatcttactactacta |
| | | ctacggtatggacgtctggggccaagggaccacggtcaccgtctcctca (SEQ ID NO: 77) |
| P03 | IGHV1- | caggtgcagctggtgcagtctggggctgaggtgaagaagcctggggcctcagtgaaggtctcctg |
| | 8 | caaggcttctggatacaccttcaccagttatgatatcaactgggtgcgacaggccactggacaag |
| | | ggcttgagtggatgggatggatgaaccctaacagtggtaacacaggctatgcacagaagttcca |
| | | gggcagagtcaccatgaccaggaacacctccataagcacagcctacatggagctgagcagcct |
| | | gagatctgaggacacggccgtgtattactgtgcgagaggcctccggattgtagtagtaccagctg |
| | | catacaacggggattactactactactacggtatggacgtctggggccaagggaccacggtcac |
| | | cgtctcctcag (SEQ ID NO: 78) |
| P04 | IGHV1- | caggttcagctggtgcagtctggagctgaggtgaagaagcctggggcctcagtgaaggtctcctg |
| | 18 | caaggcttctggttacacctttaccagctacggtatcagctgggtgcgacaggcccctggacaag |
| | | ggcttgagtggatgggatggatcagcgcttacaatggtaacacaaactatgcacagaagctcca |
| | | gggcagagtcaccatgaccacagacacatccacgagcacagcctacatggagctgaggagcct |
| | | gagatctgacgacacggccgtgtattactgtgcgagagtgaatgggtgggatgtagtagtaccag |
| | | ccactatacccgccgagagctactactactactacggtatggacgtctggggccaagggaccac |
| | | ggtcaccgtctcctca (SEQ ID NO: 79) |
| P05 | IGHV1- | caggtccagctggtacagtctggggctgaggtgaagaagcctggggcctcagtgaaggtctcctg |
| | 24 | caaggtttccggatacaccctcactgaattatccatgcactgggtgcgacaggctcctggaaaag |
| | | ggcttgagtggatgggaggttttgatcctgaagatggtgaaacaatctacgcacagaagttccag |
| | | ggcagagtcaccatgaccgaggacacatctacagacacagcctacatggagctgagcagcctg |
| | | agatctgaggacacggccgtgtattactgtgcaacagcaggctattttgactggttccattttgac |
| | | tactggggccagggaaccctggtcaccgtctcctcag (SEQ ID NO: 80) |
| P06 | IGHV1- | cagatgcagctggtgcagtctggggctgaggtgaagaagactgggtcctcagtgaaggtttcctg |
| | 45 | caaggcttccggatacaccttcacctaccgctacctgcactgggtgcgacaggcccccggacaa |
| | | gcgcttgagtggatgggatggatcacacctttcaatggtaacaccaactacgcacagaaattcca |
| | | ggacagagtcaccattaccagggacaggtctatgagcacagcctacatggagctgagcagcctg |
| | | agatctgaggacacagccatgtattactgtgcaagcgatataggggggagggctattactatggt |
| | | tcggggagttaactactactacggtatggacgtctggggccaagggaccacggtcaccgtctcct |
| | | ca (SEQ ID NO: 81) |
| P07 | IGHV1- | caggtgcagctggtgcagtctggggctgaggtgaagaagcctggggcctcagtgaaggtttcctg |
| | 46 | caaggcatctggatacaccttcaccagctactatatgcactgggtgcgacaggcccctggacaa |
| | | gggcttgagtggatgggaataatcaaccctagtggtggtagcacaagctacgcacagaagttcc |
| | | agggcagagtcaccatgaccagggacacgtccacgagcacagtctacatggagctgagcagcc |
| | | tgagatctgaggacacggccgtgtattactgtgcgagagatattgtagtggtggtagctgcgctag |
| | | aggcggggtggttcgacccctggggccagggaaccctggtcaccgtctcctcag (SEQ ID |
| | | NO: 82) |
| P08 | IGHV1- | Caaatgcagctggtgcagtctgggcctgaggtgaagaagcctgggacctcagtgaaggtctcct |
| | 58 | gcaaggcttccggattcacttttactgactctgctgtgcagtgggtgcgtcaggctcgtggacaac |
| | | gccttgagtggataggatggatcgtcgttggcagtgctaacacaaactacgcacagaagttccag |
| | | gagagagtcaccattaccagggacatgtccacaagcacagcctacatggagctgagcagcctg |
| | | agatccgaggacacggccgtctattactgtgcggcagggggtgtgggtggattctggacttcttac |
| | | tactactactacatggacgtctggggcaaagggaccacggtcaccgtctcctca (SEQ ID |
| | | NO: 83) |
| P09 | IGHV1- | Caggtgcagctggtgcagtctggggctgaggtgaagaagcctgggtcctcggtgaaggtctcctg |
| | 69/ | caaggcttctggaggcaccttcagcagctatgctatcagctgggtgcgacaggcccctggacaa |
| | IGHV1- | gggcttgagtggatgggagggatcatccctatctttggtacagcaaactacgcacagaagttcca |
| | 69D | gggcagagtcacgattaccgcggacgaatccacgagcacagcctacatggagctgagcagcct |
| | | gagatctgaggacacggccgtgtattactgtgcgacgacgtattactatgatagtagtggttatta |
| | | ctgggttgatgactactactacggtatggacgtctggggccaagggaccacggtcaccgtctcct |
| | | ca (SEQ ID NO: 84) |
| P10 | IGHV1- | gaggtccggctggtacaatctggggctgaggtgaagaagcctggggctacagtgaaaatctcctg |
| | 69-2 | caaggtttctggatacaccttcaccgactactacatccactgggtgcgacaggcccctggaaaag |
| | | ggcttgagtggatgggacttgttgatcctgaagatggtgaaacaacatacgcagagaagttccag |
| | | ggcagagtcaccataaccgcggacacgtctacagacacagcctatatggaggtgcgcagcctga |
| | | gatctgaagacacggccgtgtattattgtgcaaccattttgagtgattatttggttgacttctgggg |
| | | ccagggaaccct (SEQ ID NO: 85) |
| P11 | IGHV2- | cagatcaccttgaaggagtctggtcctacgctggtgaaacccacacagaccctcacgctgacct |
| | 5 | gcaccgtctctgggttctcagtcagcgctagtggagtgagtgtgggctggatccgtcagccccca |
| | | ggaaaggccctggaatggcttgcactcatttattgggatgatgataagcgctacagcccgtctct |
| | | gaaaagcaggctcaccatcaccaaggacatctccaaaaaccacgtggtccttacaatgaccaa |
| | | catggaccctatggacacagccacatattattgtgcacgcagggcggggtatgactggaattacg |
| | | agcgtgcctggttcgacccctggggccagggaatcccggtcaccgtctcctcag (SEQ ID |
| | | NO: 86) |
| P12 | IGHV2- | caggtcaccttgagggagtctggtcctgtgctggtgaaacccacagagaccctcacgctgacctg |
| | 26 | caccgtctctgggttctcactcagcaatgttggaatgggtgtgagctggatccgtcagcccccag |
| | | ggaaggccctggaatggcttgcacacattttttcgaatgacgaaaaatcctgcagcacatctctg |
| | | aagagcaggctcaccatctccaaggacacctccaaaagccacgtggtccttatcatgaccaaca |
| | | tggaccctgtggacacagccacatattactgtgcacggagtagtagtatcagcgatgatgcttttg |
| | | atgtctggggccaagggacaatggtcaccgtctcctcag (SEQ ID NO: 87) |
| P13 | IGHV2- | caggtcaccttgagggagtctggtcctgcgctggtgaaacccacacagaccctcacactgacct |
| | 70/ | gcaccttctctgggttctcactcagcactagtggaatgtgtgtgagctggatccgtcagcccccag |
| | IGHV2- | ggaaggccctggagtggcttgcactcattgattgggatgatgataaatactacagcacatctctg |
| | 70D | aagaccaggctcaccatctccaaggacacctccaaaaaccaggtggtccttacaatgaccaac |
| | | atggaccctgtggacacagccacgtattactgtgcacggatacggtatagcagcagctggtcccc |
| | | agggccttggtactactactacggtatggacgtctggggccaagggaccacggtcaccgtctcct |
| | | cag (SEQ ID NO: 88) |
| P14 | IGHV3- | gaggtgcagctggtggagtctgggggaggcttggtccagcctggggggtccctgagactctcctg |
| | 7 | tgcagcctctggattcacctttagtagctattggatgagctgggtccgccaggctccagggaagg |
| | | ggctggagtgggtggccaacataaagcaagatggaagtgagaaatactatgtggactctgtgaa |
| | | gggccgattcaccatctccagagacaacgccaagaactcactgtatctgcaaatgaacagcctg |
| | | agagccgaggacacggctgtgtattactgtgcgagagaacccgattggggaatcagatcattact |
| | | atggttcggggagttatttgggcccgatgcttttgatatctggggccaagggacaatggtcaccgt |
| | | ctcctcag (SEQ ID NO: 89) |
| P15 | IGHV3- | gaagtgcagctggtggagtctgggggaggcttggtacagcctggcaggtccctgagactctcctg |
| | 9 | tgcagcctctggattcacctttgatgattatgccatgcactgggtccggcaagctccagggaagg |
| | | gcctggagtgggtctcaggtattagttggaatagtggtagcataggctatgcggactctgtgaagg |
| | | gccgattcaccatctccagagacaacgccaagaactccctgtatctgcaaatgaacagtctgag |
| | | agctgaggacacggccttgtattactgtgcaaaagcggccccggaagaccactattgtagtggtg |
| | | gtagctgctaccccaattactactactactacggtatggacgtctggggccaagggaccacggtc |
| | | accgtctcctca (SEQ ID NO: 90) |
| P16 | IGHV3- | caggtgcagctggtggagtctgggggaggcttggtcaagcctggagggtccctgagactctcctg |
| | 11 | tgcagcctctggattcaccttcagtgactactacatgagctggatccgccaggctccagggaagg |
| | | ggctggagtgggtttcatacattagtagtagtggtagtaccatatactacgcagactctgtgaagg |
| | | gccgattcaccatctccagggacaacgccaagaactcactgtatctgcaaatgaacagcctgag |
| | | agccgaggacacggccgtgtattactgtgcgagagcccactttcccctctccgtttgggagtggt |
| | | cccatagctactactacggtatggacgtctggggccaagggaccacggtcaccgtctcctca |
| | | (SEQ ID NO: 91) |
| P17 | IGHV3- | Gaggtgcagctggtggagtctgggggaggcttggtacagcctggggggtccctgagactctcctg |
| | 13 | tgcagcctgtggattcaccttcagtagctacgacatgcactgggtccgccaagctacaggaaaa |
| | | ggtctggagtgggtctcagctattggtactgctggtgacacatactatccaggctccgtgaagggc |
| | | caattcaccatctccagagaaaatgccaagaactccttgtatcttcaaatgaacagcctgagag |
| | | ccggggacacggctgtgtattactgtgcaagagatggacatgatatggggaactactggggccg |
| | | gggaaccctggtcaccctctcctca (SEQ ID NO: 92) |
| P18 | IGHV3- | gaggtgcagctggtggagtctgggggaggcttggtaaagcctggggggtcccttagactctcctg |
| | 15 | tgcagcctctggtttcactttcagtaacgcctggatgaactgggtccgccaggctccagggaagg |
| | | ggctggagtgggtcggccgtattaaaagcaaaactgatggtgggacaacagactacgctgcacc |
| | | cgtgaaaggcagattcaccatctcaagagatgattcaaaaaacacgctgtatctgcaaatgaac |
| | | agcctgaaaaccgaggacacagccgtgtattactgtaccacagtacgtttaaacgagaacgatt |
| | | tttggagtggtaattttgaggtccactactactacggtatggacgtctggggccaagggaccacg |
| | | gtcaccgtctcctca (SEQ ID NO: 93) |
| P19 | IGHV3- | gaggtgcagctggtggagtctgggggaggtgtggtacggcctggggggtccctgagactctcctg |
| | 20 | tgcagcctctggattcacctttgatgattatgccatgagctgggtccgccaagctccaggggcgg |
| | | ggctggagtgggtctctggtattcattggaacggtggtggcacaggttatgcagactctgtgcagg |
| | | gccgattcaccatctccagagacaacgccaagaactccctgtatttgcaaatgaacagtctgag |
| | | agtcgaagacacggccttctattactgtgcgagagttggcggtgggggtatagcagttgctggta |
| | | caaactggctcgacccctggggccagggaatcctggtcaccgtctcctcag (SEQ ID |
| | | NO: 94) |
| P20 | IGHV3- | gaggtgcagctggtggagtctgggggaggcctggtcaagcctggggggtccctgagactctcctg |
| | 21 | tgcagcctctggattcaccttcagtagctatagcatgaactgggtccgccaggctccagggaagg |
| | | ggctggagtgggtctcatccattagtagtagtagtagttacatatactacgcagactcagtgaag |
| | | ggccgattcaccatctccagagacaacgccaagaactcactgtatctgcaaatgaacagcctga |
| | | gagccgaggacacggctgtgtattactgtgcgaggaaatactatgatagtagtggttattactact |
| | | ggcaagactactactacggtatggacgtctggggccaagggaccacggtcaccgtctcctca |
| | | (SEQ ID NO: 95) |
| P21 | IGHV3- | gaggtgcagctgttggagtctgggggaggcttggtacagcctggggggtccctgagactctcctg |
| | 23/ | tgcagcctctggattcacctttagcagctatgccatgagctgggtccgccaggctccagggaagg |
| | IGHV3- | ggctggagtgggtctcagctattagtggtagtggtggtagcacatactacgcagactccgtgaag |
| | 23D | ggccggttcaccatctccagagacaattccaagaacacgctgtatctgcaaatgaacagcctga |
| | | gagccgaggacacggccgtatattactgtgcgaaagattcggggatgcagtattacgatttttgg |
| | | agtggttatcaccccgcctactactactactacggtatggacgtctggggccaagggaccacggt |
| | | caccgtctcctca (SEQ ID NO: 96) |
| P22 | IGHV3- | caggtgcagctggtggagtctgggggaggcgtggtccagcctgggaggtccctgagactctcctg |
| | 30/ | tgcagcctctggattcaccttcagtagctatggcatgcactgggtccgccaggctccaggcaagg |
| | IGHV3- | ggctggagtgggtggcagttatatcatatgatggaagtaataaatactatgcagactccgtgaag |
| | 30-5 | ggccgattcaccatctccagagacaattccaagaacacgctgtatctgcaaatgaacagcctga |
| | | gagctgaggacacggctgtgtattactgtgcgaaagggattcgaaggcttccttattactatgata |
| | | gtagtggtccgttcgacccctggggccagggaaccctggtcaccgtctcctcag (SEQ ID |
| | | NO: 97) |
| P23 | IGHV3- | caggtgcagctggtggagtctgggggaggcgtggtccagcctgggaggtccctgagactctcctg |
| | 30-3 | tgcagcctctggattcaccttcagtagctatgctatgcactgggtccgccaggctccaggcaagg |
| | | ggctggagtgggggcagttatatcatatgatggaagcaataaatactacgcagactccgtgaag |
| | | ggccgattcaccatctccagagacaattccaagaacacgctgtatctgcaaatgaacagcctga |
| | | gagctgaggacacggctgtgtattactgtgcgagagattttttgcagtggctggtacctggtgcat |
| | | attactactactactacggtatggacgtctggggccaagggaccacggtcaccgtctcctcag |
| | | (SEQ ID NO: 98) |
| P24 | IGHV3- | caggtgcagctggtggagtctgggggaggcgtggtccagcctgggaggtccctgagactctcctg |
| | 33 | tgcagcgtctggattcaccttcagtagctatggcatgcactgggtccgccaggctccaggcaagg |
| | | ggctggagtgggtggcagttatatggtatgatggaagtaataaatactatgcagactccgtgaag |
| | | ggccgattcaccatctccagagacaattccaagaacacgctgtatctgcaaatgaacagcctga |
| | | gagccgaggacacggctgtgtattactgtgcgagagatccgggtttgtacggttgcctggctagt |
| | | agtaccagctgctatccctttgactactggggccagggaaccctggtcaccgtctcctcag |
| | | (SEQ ID NO: 99) |
| P25 | IGHV3- | gaggtgcagctggtggagtctcggggagtcttggtacagcctggggggtccctgagactctcctg |
| | 38-3 | tgcagcctctggattcaccgtcagtagcaatgagatgagctgggtccgccaggctccagggaag |
| | | ggtctggagtgggtctcatccattagtggtggtagcacatactacgcagactccaggaagggcag |
| | | attcaccatctccagagacaattccaagaacacgctgcatcttcaaatgaacagcctgagagct |
| | | gaggacacggctgtgtattactgtacggggtggttcgggaggatactccaatatggctactgggg |
| | | ccagggaaccctggtcaccgtctcctcag (SEQ ID NO: 100) |
| P26 | IGHV3- | gaagtgcagatggtggaatctggaggaggcgtggagcagcctggggggtccctgagactctcct |
| | 43/ | gcgcaacgtctggattcaactttgatgattattccatgcactgggtccgtcaagctccagggaag |
| | IGHV3- | ggtctggagtggatctctcttattactggggacggtgataccacatcctatgcagactctgtgaag |
| | 43D | gacagattcatcatctccagagacaaccgcaaaaactccctgtatctgcaaatgtacagtctga |
| | | caattgaggacaccgccttctattattgtacacgaggcggaattaaaaaggccgatagtgggag |
| | | ctactatcgatggctcggcccctggggccagggaaccctggtcaccgtctcctca (SEQ ID |
| | | NO: 101) |
| P27 | IGHV3- | gaggtgcagctggtggagtctgggggaggcttggtacagcctggagggtccctgagactctcctg |
| | 48 | tgcagcctctggattcaccttcagtagttatgaaatgaactgggtccgccaggctccagggaagg |
| | | ggctggagtgggtttcatacattagtagtagtggtagtaccatatactacgcagactctgtgaagg |
| | | gccgattcaccatctccagagacaacgccaagaactcactgtatctgcaaatgaacagcctgag |
| | | agccgaggacacggctgtttattactgtgcgagaggctacgatttttggagtggttacctctcata |
| | | ctactactactacggtatggacgtctggggccaagggaccacggtcaccgtctcctca (SEQ |
| | | ID NO: 102) |
| P28 | IGHV3- | gaggtgcagctggtggagtctgggggaggcttggtaaagccagggcggtccctgagactctcctg |
| | 49 | tacagcttctggattcacctttggtgattatgctatgagctggttccgccaggctccagggaaggg |
| | | gctggagtgggtaggtttcattagaagcaaagcttatggtgggacaacagaatacgccgcgtctg |
| | | tgaaaggcagattcaccatctcaagagatgattccaaaagcatcgcctatctgcaaatgaacag |
| | | cctgaaaaccgaggacacagccgtgtattactgtactatatcggatatggttcattacgatttttg |
| | | gagtggttactattcggggggctggttcgacccctggggccagggaaccctggtcaccgtctcct |
| | | ca (SEQ ID NO: 103) |
| P29 | IGHV3- | gaggtgcagctggtggagtctggaggaggcttgatccaccctggggggtccctgagactctcttg |
| | 53 | tgcagcctctgggttcaccgtcagtcacaacgacatgagctgggtccgccaggctccagggaag |
| | | gggctggagtgggtctcagttatttatagcggtggtaacacatactacgcagactccgtgaaggg |
| | | ccgattcaccatctccagagacaattccaagaacacgctggatcttcaaatgaacagcctgaga |
| | | gccgaggacacggccgtgtattactgtgcgagaacgcctgggggtagtggttatattgactactg |
| | | gggccagggaaccctggtcaccgtctcctcag (SEQ ID NO: 104) |
| P30 | IGHV3- | gaggtgcagctggtggagtctgggggaggcttggtccagcctggggggtccctgagactctcttgt |
| | 64/ | tcagcctctggattccccttcagtacctatgctatccactgggtccgccaggctccagggaaggg |
| | IGHV3- | actggagtatgtttcagctattaatagtgatgggggtagcacctactacgcagactccgtgaagg |
| | 64D | acagattcaccatctccagagacaattccaagaacactctgtatcttcaaatgaacagtctgaga |
| | | actgaggacacggctgtctattactgtgttaaaggtgacatcggggaattatcaatattatactac |
| | | tactacggtttgcacgtctggggccaggggaccgcggtcaccgtctcctcag (SEQ ID |
| | | NO: 105) |
| P31 | IGHV3- | Gaggtgcagctggtggagtctgggggaggcttggtccagcctggggggtccctgagactctcctg |
| | 66 | tgcagcctctggattcaccgtcagcagcaactacatgaactgggtccgccaggctccagggaag |
| | | gggctggagtgggtctcagttatttatagcggtggtagcacatactacgcagactccgtgaaggg |
| | | cagattcaccatctccagagacaattccaacaacacgctgtatcttcaaatgaacagcctgaga |
| | | gccgaggacacggctgtgtatttctgtgcgagtgtcctcaccgagaactggtacttcgatctctgg |
| | | ggccgtggcaccctggtcaccgtctcctcag (SEQ ID NO: 106) |
| P32 | IGHV3- | gaggtgcaactggtggagtctgggggaggcttggtccagcctggagggtccctgatactctcctg |
| | 72 | tgcttcctctggattcaccttcagtgaccactacatggactgggtccgccaggctccagggaagg |
| | | ggctggagtgggttggccgtagtggaaacaaagataatcgttctaccacaagatacgccgcgtct |
| | | gtggagggcagattcaccatctcaagagatgaatcgaagaacttagtatatttgcaaatgaaca |
| | | acctgaaaagcgaagacacggccatatattactgtactagagataaaggtcatctctgctgggg |
| | | ccagggaaccctggtcaccgtctcctcag (SEQ ID NO: 107) |
| P33 | IGHV3- | gaggtgcagctggtggagtccgggggaggcttggtccagcctggggggtccctgaaactctcctg |
| | 73 | tgcagcctctgggttcaccttcagtggctctgctatgcactgggtccgccaggcttccgggaaagg |
| | | gctggagtgggttggccgtattagaagcaaagctaacagttacgcgacagcatatgctgcgtcgg |
| | | tgaaaggcaggttcaccatctccagagatgattcaaagaacacggcgtatctgcaaatgaacag |
| | | cctgaaaaccgaggacacggccgtgtattactgtacatacgatttttggagtggttattgggtggg |
| | | tgactactactactacggtatggacgtctggggccaagggaccacggtcaccgtctcctcag |
| | | (SEQ ID NO: 108) |
| P34 | IGHV3- | gaggtgcagctggtggagtccgggggaggcttagttcagcctggggggtccctgagactctcctg |
| | 74 | tgcagcctctggattcaccttcagtagctactggatgcactgggtccgccaagctccagggaagg |
| | | ggctggtgtgggtctcacgtattaatagtgatgggagtagcacaagctacgcggactccgtgaag |
| | | ggccgattcaccatctccagagacaacgccaagaacacgctgtatctgcaaatgaacagtctga |
| | | gagccgaggacacggctgtgtattactgtgcaagccccggagagtattatgtgccgtcagactac |
| | | tactactacggtatggacgtctggggccaagggaccacggtcaccgtctcctca (SEQ ID |
| | | NO: 109) |
| P35 | IGHV4- | caggtgcagatgcaggagtcgggcccaggaatggtgaagccttcggagaccctgtccctcactt |
| | 4 | gcactgtctctggtggctccatcagtagttactactggagctggatccggcagcccgccgggaag |
| | | ggactggagtggattgggcgtatctataccagtgggagcaccaactacaacccctccctcaaga |
| | | gtcgagtcaccatgtcagtagacacgtccaagaaccagttctccctgaagctgagctctgtgacc |
| | | gccgcggacacggccgtgtattactgtgcgagagatctcaggttgggggtagctgctacgatccc |
| | | atacggtatggacgtctggggccaagggacc (SEQ ID NO: 110) |
| P36 | IGHV4- | Cagctgcagctgcaggagtccggctcaggactggtgaagccttcacagaccctgtccctcacct |
| | 30-2 | gcgctgtctctggtggctccatcagcagtggtggttactcctggagctggatccggcagccacca |
| | | gggaagggcctggagtggattgggtacatctatcatagtgggagcacctactacaacccgtccct |
| | | caagagtcgagtcaccatatcagtagacaggtccaagaaccagttctccctgaagctgagctct |
| | | gtgaccgccgcggacacggccgtgtattactgtgccagaggccaatacaattactatgatagtag |
| | | tggttattactacgatagacagtactactttgactactggggccagggaaccctggtcaccgtctc |
| | | ctcag (SEQ ID NO: 111) |
| P37 | IGHV4- | caggtgcagctgcaggagtcgggcccaggactggtgaagccttcacagaccctgtccctcacct |
| | 30-4 | gcactgtctctggtggctccatcagcagtggtgattactactggagttggatccgccagccccca |
| | | gggaagggcctggagtggattgggtacatctattacagtgggagcacctactacaacccgtccct |
| | | caagagtcgagttaccatatcagtagacacgtccaagaaccagttctccctgaagctgagctctg |
| | | tgactgccgcagacacggccgtgtattactgtgccagagaggggggggggtattacgatttttgg |
| | | agtggttattacggtatggacgtctggggccaagggaccacggtcaccgtctcctca (SEQ ID |
| | | NO: 112) |
| P38 | IGHV4- | caggtgcagctgcaggagtcgggcccaggactggtgaagccttcacagaccctgtccctcacct |
| | 31/ | gcactgtctctggtggctccatcagcagtggtggttactactggagctggatccgccagcaccca |
| | IGHV4- | gggaagggcctggagtggattgggtacatctattacagtgggagcacctactacaacccgtccct |
| | 30-1 | caagagtcgagttaccatatcagtagacacgtctaagaaccagttctccctgaagctgagctctg |
| | | tgactgccgcggacacggccgtgtattactgtgcgagagtgggggattactatgatagtagtggtt |
| | | attaccccggatcgtactactttgactactggggccagggaaccctggtcaccgtctcctcag |
| | | (SEQ ID NO: 113) |
| P39 | IGHV4- | caggtgcagctacagcagtggggcgcaggactgttgaagccttcggagaccctgtccctcacctg |
| | 34 | cgctgtctatggtgggtccttcagtggttactactggagctggatccgccagcccccagggaagg |
| | | ggctggagtggattggggaaatcaatcatagtggaagcaccaactacaacccgtccctcaagag |
| | | tcgagtcaccatatcagtagacacgtccaagaaccagttctccctgaagctgagctctgtgaccg |
| | | ccgcggacacggctgtgtattactgtgcgagacgcctacgctattacgatattttgactggttacc |
| | | cgactcttgactactggggccagggaaccctggtcaccgtctcctcag (SEQ ID NO: 114) |
| P40 | IGHV4- | caggtgcagctgcaggagtcgggcccaggactggtgaagccttcggagaccctgtccctcacct |
| | 38-2 | gcgctgtctctggttactccatcagcagtggttactactggggctggatccggcagcccccaggg |
| | | aaggggctggagtggattgggagtatctatcatagtgggagcacctactacaacccgtccctcaa |
| | | gagtcgagtcaccatatcagtagacacgtccaagaaccagttctccctgaagctgagctctgtga |
| | | ccgccgcagacacggccgtgtattactgtgcgagacttaaggattacgatttttggagtggttatt |
| | | atacggggtatggtgcttttgatatctggggccaagggacaatggtcaccgtctcctcag (SEQ |
| | | ID NO: 115) |
| P41 | IGHV4- | cagctgcagctgcaggagtcgggcccaggactggtgaagccttcggagaccctgtccctcacct |
| | 39 | gcactgtctctggtggctccatcagcagtagtagttactactggggctggatccgccagccccca |
| | | gggaaggggctggagtggattgggagtatctattatagtgggagcacctactacaacccgtccct |
| | | caagagtcgagtcaccatatccgtagacacgtccaagaaccagttctccctgaagctgagctct |
| | | gtgaccgccgcagacacggctgtgtattactgtgcgagccaaaccgggtatagcagcagctggt |
| | | acacccggagctggttcgacccctggggccagggaaccctggtcaccgtctcctcag (SEQ ID |
| | | NO: 116) |
| P42 | IGHV4- | caggtgcagctgcaggagtcgggcccaggactggtgaagccttcggagaccctgtccctcacct |
| | 59 | gcactgtctctggtggctccatcagtagttactactggagctggatccggcagcccccagggaag |
| | | ggactggagtggattgggtatatctattacagtgggagcaccaactacaacccctccctcaagag |
| | | tcgagtcaccatatcagtagacacgtccaagaaccagttctccctgaagctgagctctgtgaccg |
| | | ctgcggacacggccgtgtattactgtgcgagagcccgcgaccccatgtattacgatttttggagtg |
| | | gttattataccggtatcttgggctttgactactggggccagggaaccctggtcaccgtctcctcag |
| | | (SEQ ID NO: 117) |
| P43 | IGHV4- | caggtgcagctgcaggagtcgggcccaggactggtgaagccttcggagaccctgtccctcacct |
| | 61 | gcactgtctctggtggctccgtcagcagtggtagttactactggagctggatccggcagccccca |
| | | gggaagggactggagtggattgggtatatctattacagtgggagcaccaactacaacccctccct |
| | | caagagtcgagtcaccatatcagtagacacgtccaagaaccagttctccctgaagctgagctct |
| | | gtgaccgctgcggacacggccgtgtattactgtgcgagaaccctatattacgatttttggagtggg |
| | | cctcactactacggtatggacgtctggggccaagggaccacggtcaccgtctcctca (SEQ ID |
| | | NO: 118) |
| P44 | IGHV5- | Gaagtgcagctggtgcagtctggagcagaggtgaaaaagcccggggagtctctgaggatctcct |
| | 10-1 | gtaagggttctggatacagctttaccagctactggatcagctgggtgcgccagatgcccgggaaa |
| | | ggcctggagtggatggggaggattgatcctagtgactcttataccaactacagcccgtccttcca |
| | | aggccacgtcaccatctcagctgacaagtccatcagcactgcctacctgcagtggagcagcctg |
| | | aaggcctcggacaccgccatgtattactgtgcgatcccggtagtaccagctgctatggggggagt |
| | | actggggccagggaaccctggtcaccgtctcctcag (SEQ ID NO: 119) |
| P45 | IGHV5- | gatgtgcagctggtgcagtctggagcagaggtgaaaaagcccggggagtctctaaggatctcctg |
| | 51 | taaggcttctggacacagcttgaccgattactgggtcacctgggtgcgccagatgcccgggaaag |
| | | gcctggagtggatgggggtcatctatcctgctgactctactacaacatacaacccgtccttccaa |
| | | ggccaggtcagtatctcagccgacaagtccatcagcaccgcctacctgcagtggagcagcctga |
| | | aggcctcggacagcgccatgtattactgtgcgagggttcgaggtggaaacagtgactggtaccac |
| | | cagggctactttgactactggggccagggagccctggtcaccgtctcctcag (SEQ ID |
| | | NO: 120) |
| P46 | IGHV6- | caggtacagctgcagcagtcaggtccaggactggtgacgccctcgcagaccctctcactcacct |
| | 1 | gtgccatctccggggacagtgtctctagcaatagtgttgtttggaactggatcaggcagtccccat |
| | | cgagaggccttgagtggctgggaaggacatactacaggtccaagtggtatagtgattatgcagtg |
| | | gctgtgagaagtcgaatatccatcaacccagacacatccaagaaccagttctctctgcagctga |
| | | actctgtgactcccgaagacacggctgtttattactgtgcaagatacattggttactactactttg |
| | | accagtgggcccagggaaccctggtcaccgtctcctcag (SEQ ID NO: 121) |
| P47 | IGHV7- | caggtgcagctggtgcaatctgggtctgagttgaagaagcctggggcctcagtgaaggtttcctg |
| | 4-1 | caaggcttctggatacaccttcactagctatgctatgaattgggtgcgacaggcccctggacaag |
| | | ggcttgagtggatgggatggatcaacaccaacactgggaacccaacgtatgcccagggcttcac |
| | | aggacggtttgtcttctccttggacacctctgtcagcacggcatatctgcagatcagcagcctaa |
| | | aggctgaggacactgccgtgtattactgtgcgagagagcagtggctgccccagggcaactttgac |
| | | tactggggccagggaaccctggtcaccgtctcctcag (SEQ ID NO: 122) |

Each of SEQ ID NO:76 to SEQ ID NO:122 is preferably included in a distinct plasmid, the internal control then comprising plasmids comprising respectively the sequences SEQ ID NO:76 to SEQ ID NO: 122.

Any suitable plasmid may be used, such as pCR 2.1-TOPO (TOPO TA Cloning Kit, ThermoFischer Scientific).

Such an internal control is useful to check that the amplification has been efficient for all possible functional IGHV genes and that all primers included in the kits work correctly (see FIG. 5).

### Preferred Kits According to the Invention

Preferred kits according to the invention include:
a) A kit for determination of CLL mutational status from gDNA using NGS sequencing, comprising:
   i) forward primers consisting respectively, from 5′ to 3′, of a first adapter sequence (preferably of structure 5′-Forward flow cell binding adapter-Index i5-Forward sequencing primer site-3′, wherein the Forward flow cell binding adapter is of sequence SEQ ID NO:34, the Forward sequencing primer site is of sequence SEQ ID NO:35, and the Index i5 is selected from one of the sequences SEQ ID NO:36 to 53 of Table 7 above) fused to one of the sequences SEQ ID NO:1 to SEQ ID NO:23, preferably provided as one or more separated forward primers mixtures differing by their index part (preferably by index i5),
   ii) one or more reverse primer(s) each consisting, from 5′ to 3′, of a second adapter sequence (preferably of structure 5′-Reverse flow cell binding adapter-Index i7—Reverse sequencing primer site-3′, wherein the Reverse flow cell binding adapter is of sequence SEQ ID NO:54, the Reverse sequencing primer site is of sequence SEQ ID NO:55, and the Index i7 is selected from one of the sequences SEQ ID NO:56 to 75 of Table 8 above) fused to SEQ ID NO:30, preferably provided as one or more separated reverse primers mixtures differing by their index part (preferably by index i7), and
   iii) optionally, plasmids comprising respectively sequences SEQ ID NO:76 to SEQ ID NO:122;
b) A kit for determination of CLL mutational status from cDNA using NGS sequencing, comprising:
   i) forward primers consisting respectively, from 5′ to 3′, of a first adapter sequence (preferably of structure 5′-Forward flow cell binding adapter-Index i5-Forward sequencing primer site-3′, wherein the Forward flow cell binding adapter is of sequence SEQ ID NO:34, the Forward sequencing primer site is of sequence SEQ ID NO:35, and the Index i5 is selected from one of the sequences SEQ ID NO:36 to 53 of Table 7 above) fused to one of the sequences SEQ ID NO:24 to SEQ ID NO:29 (and optionally SEQ ID NO:133), preferably provided as one or more separated forward primers mixtures differing by their index part (preferably by index i5),
   ii) reverse primers consisting respectively, from 5′ to 3′, a second adapter sequence (preferably of structure 5′-Reverse flow cell binding adapter-Index i7—Reverse sequencing primer site-3′, wherein the Reverse flow cell binding adapter is of sequence SEQ ID NO:54, the Reverse sequencing primer site is of sequence SEQ ID NO:55, and the Index i7 is selected from one of the sequences SEQ ID NO:56 to 75 of Table 8 above) fused to one of the sequences SEQ ID NO:31 to SEQ ID NO:33 (and optionally SEQ ID NO:134), preferably provided as one or more separated reverse primers mixtures differing by their index part (preferably by index i7), and
   iii) optionally, plasmids comprising respectively sequences SEQ ID NO:76 to SEQ ID NO:122;
c) A kit for determination of CLL mutational status from gDNA and cDNA using NGS sequencing, comprising:
   i) forward primers consisting respectively, from 5′ to 3′, of a first adapter sequence (preferably of structure 5′-Forward flow cell binding adapter-Index i5-Forward sequencing primer site-3′, wherein the Forward flow cell binding adapter is of sequence SEQ ID NO:34, the Forward sequencing primer site is of sequence SEQ ID NO:35, and the Index i5 is selected from one of the sequences SEQ ID NO:36 to 53 of Table 7 above) fused to one of the sequences SEQ ID NO:1 to SEQ ID NO:23, preferably provided as one or more separated forward primers mixtures differing by their index part (preferably by index i5),
   ii) one or more reverse primer(s) each consisting, from 5′ to 3′, of a second adapter sequence (preferably of structure 5′-Reverse flow cell binding adapter-Index i7—Reverse sequencing primer site-3′, wherein the Reverse flow cell binding adapter is of sequence SEQ ID NO:54, the Reverse sequencing primer site is of sequence SEQ ID NO:55, and the Index i7 is selected from one of the sequences SEQ ID NO:56 to 75 of Table 8 above) fused to SEQ ID NO:30, preferably provided as one or more separated reverse primers mixtures differing by their index part (preferably by index i7),
   iii) forward primers consisting respectively, from 5′ to 3′, of a third adapter sequence (preferably of structure 5′-Forward flow cell binding adapter-Index i5-Forward sequencing primer site-3′, wherein the Forward flow cell binding adapter is of sequence SEQ ID NO:34, the Forward sequencing primer site is of sequence SEQ ID NO:35, and the Index i5 is selected from one of the sequences SEQ ID NO:36 to 53 of Table 7 above) fused to one of the sequences SEQ ID NO:24 to SEQ ID NO:29 (and optionally SEQ ID NO:133), preferably provided as one or more separated forward primers mixtures differing by their index part (preferably by index i5),
   iv) reverse primers consisting respectively, from 5′ to 3′, a fourth adapter sequence (preferably of structure 5′-Reverse flow cell binding adapter-Index i7—Reverse sequencing primer site-3′, wherein the Reverse flow cell binding adapter is of sequence SEQ ID NO:54, the Reverse sequencing primer site is of sequence SEQ ID NO:55, and the Index i7 is selected from one of the sequences SEQ ID NO:56 to 75 of Table 8 above) fused to one of the sequences SEQ ID NO:31 to SEQ ID NO:33 (and optionally SEQ ID NO:134), preferably provided as one or more separated reverse primers mixtures differing by their index part (preferably by index i7), and
   v) optionally, plasmids comprising respectively sequences SEQ ID NO:76 to SEQ ID NO:122;
d) A kit for determination of CLL mutational status from gDNA using Sanger sequencing, comprising:
   i) forward primers consisting respectively of the sequences SEQ ID NO:24 to SEQ ID NO:29 (and optionally SEQ ID NO: 133), preferably provided as a forward primers mixture,
   ii) a reverse primer consisting of SEQ ID NO:30, and
   iii) optionally, plasmids comprising respectively sequences SEQ ID NO:76 to SEQ ID NO:122;
e) A kit for determination of CLL mutational status from cDNA using Sanger sequencing, comprising:
   i) forward primers consisting respectively of the sequences SEQ ID NO:24 to SEQ ID NO:29 (and optionally SEQ ID NO:133), preferably provided as a forward primers mixture,
   ii) reverse primers consisting respectively of the sequences SEQ ID NO:31 to SEQ ID NO:33 (and optionally SEQ ID NO:134), preferably provided as a reverse primers mixture, and
   iii) optionally, plasmids comprising respectively sequences SEQ ID NO:76 to SEQ ID NO:122;
f) A kit for determination of CLL mutational status from gDNA and cDNA using Sanger sequencing, comprising:
   i) forward primers consisting respectively of the sequences SEQ ID NO:24 to SEQ ID NO:29 (and optionally SEQ ID NO:133), preferably provided as a forward primers mixture,
   ii) a reverse primer comprising SEQ ID NO:30, preferably a reverse primer consisting of SEQ ID NO:30,
   iii) reverse primers consisting respectively of the sequences SEQ ID NO:31 to SEQ ID NO:33 (and optionally SEQ ID NO:134), preferably provided as a reverse primers mixture, and
   iv) optionally, plasmids comprising respectively sequences SEQ ID NO:76 to SEQ ID NO:122.

### Further Optional Elements of the Kits According to the Invention

Any kit according to the invention as described above may optionally further contain:
a) instructions of use for determining the mutational status of a patient suffering from CLL,
b) a high fidelity DNA polymerase (such as the Platinum® HighFidelity Taq Polymerase from Invitrogen available from Thermo Fisher Scientific), and its buffer (preferably 5× to 15×, more preferably 10×)
c) a dNTP mix (preferably 5 to 15 mM, 8 to 12 mM, more preferably 10 mM),
d) a MgSO4 solution (preferably 25 to 75 mM, 40 to 60 mM, more preferably 50 mM), and/or
e) nuclease-free water.

### Instructions of Use

The kits according to the invention are intended for determining the mutational status of a patient suffering from CLL, and may thus further contain instructions of use for determining the mutational status of a patient suffering from CLL.

Such instructions may contain:
Instructions for amplifying rearranged IGVH genes using the primers included in the kit according to the invention, including other reagents to be used and optimal amplification conditions;
Instructions for sequencing the amplified rearranged IGVH genes using NGS or Sanger sequencing;
Instructions for bioinformatic analysis of the obtained sequences; and/or
Instructions for determination of the CLL mutational status based on bioinformatic analysis of the obtained sequences.

### High Fidelity DNA Polymerase

The kits according to the invention are intended for determination of CLL mutational status, which is based on detection of the presence of somatic hypermutations in the IGHV gene compared to the closest germline IGHV gene.

As a result, amplification should preferably be performed with a “high fidelity DNA polymerase”, which is herein defined as a DNA polymerase with a low error rate, and more precisely as a DNA polymerase with an error rate at least 5 times lower than, preferably at least 10 times lower than conventional Thermus aquaticus DNA polymerase (referred to as “Taq polymerase”, see for instance U.S. Pat. No. 6,127,155, available from Roche). Preferably, a high fidelity DNA polymerase has an error rate (defined as the number of mutations per base pair per template duplication, preferably measured by direct sequencing of cloned PCR products) lower than 5×10⁻⁶, preferably lower than 4×10⁻⁶ or lower than 3×10⁻⁶.

Such high fidelity DNA polymerases are known in the art and commercially available. For instance, McInerney et al (McInerney Peter et al. Molecular Biology International. Volume 2014, Article ID 287430, 8 pages, http://dx.doi.org/10.1155/2014/287430) showed that Pfu (a high-fidelity, thermostable enzyme of approximately 90 kDa isolated from Pyrococcus furiosus strain Vc1 DSM3638, see Lundberg, K. S. et al. (1991) Gene 108, 1-6), Phusion Hot start (a Pyrococcus-like DNA polymerase proofreading enzyme extremely processive available from Finnzymes), and Pwo (a DNA polymerase which was originally isolated from Pyrococcus woesei, a hyperthermophilic archaebacterium, available from Roche) polymerases have an error rate (defined as the number of mutations per base pair per template duplication and measured by direct sequencing of cloned PCR products) lower than 3×10⁻⁶, which is more than 10 times lower than conventional Taq polymerase. High Fidelity Platinum Taq polymerase (Invitrogen, available from Thermo Fisher Scientific) was found to have a 4-fold error reduction when evaluated by sensitive NGS assay as compared to the standard Platinum Taq polymerase (Filges S. et al. (2019) Sci Rep 9, 3503).

### Methods for Determining the Mutational Status of CLL Patients Using the Kits According to the Invention

The present invention also relates to a method for determining the mutational status of a patient suffering from B-cell chronic lymphocytic leukemia (CLL) from a biological sample of said CLL patient, comprising the steps of:
a) obtaining genomic DNA (gDNA) and/or complementary DNA (cDNA) from the biological sample,
b) amplifying rearranged immunoglobulin heavy chain genes from gDNA and/or cDNA by multiplex polymerase chain reaction (PCR) using primers from the kit according to the invention;
c) sequencing amplified rearranged heavy chain immunoglobulin genes using either Sanger or NGS sequencing depending on the composition of the kit and identifying a clonal productive rearranged heavy chain immunoglobulin gene,
d) aligning the identified clonal productive rearranged heavy chain immunoglobulin gene to germline immunoglobulin IGHV, IGHD and IGHJ genes, determining the percentage of identity between the IGHV gene of the identified clonal productive rearranged heavy chain immunoglobulin gene and its closest germline immunoglobulin IGHV gene, and
e) determining the mutational status of the CLL patient, wherein the mutational status is:
   unmutated if the percentage of identity between the IGHV gene of the identified clonal productive rearranged heavy chain immunoglobulin gene and its closest germline immunoglobulin IGHV gene is equal or higher to 98% (e.g. 2% or less mutations), and
   mutated if the percentage of identity between the IGHV gene of the identified clonal productive rearranged heavy chain immunoglobulin gene and its closest germline immunoglobulin IGHV gene is below 98% (e.g. more than 2% mutations).

### Step a)

In step a), genomic DNA (gDNA) and/or complementary DNA (cDNA) is obtained from the CLL patient's biological sample.

### Biological Sample

In the context of the invention, the biological sample may be any biological sample containing CLL cells. Suitable biological samples include a blood sample, a bone marrow sample, a lymph node sample or any tissue sample infiltrated by CLL cells. Preferably, the biological sample is a blood sample.

Preferably, mononuclear cells of the biological sample are purified using conventional methods before extraction of gDNA or RNA.

gDNA may be directly extracted from the biological sample (preferably from mononuclear cells of the biological sample) using conventional methods. Schematically, extraction of gDNA comprises:
i) lysing cells of the biological sample using a lysis solution comprising a proteinase, and
ii) isolating gDNA on a silica-based matrix

Alternatively, cDNA may be obtained by:
i) Extracting RNA from the biological sample by:
   a. lysing cells of the biological sample using a lysis solution comprising a chaotropic agent,
   b. isolating RNA on a silica-based matrix, and
ii) Converting RNA to cDNA using a reverse transcriptase.

The above methods are conventional and well-known to those skilled in the art.

Preferred Division of the Biological Sample into 2 Parts

As already explained above, the inventors have developed a methodology resulting in a very high rate of success in determination of the IGHV mutational status in CLL, which relies on PCR-based assays allowing the amplification of the entire IGHV regions, starting from gDNA and, only if no clonal productive rearranged heavy chain immunoglobulin gene is sequenced from gDNA, further analyzing cDNA of the same patient. This necessarily involves dividing the CLL patient's biological sample into 2 parts, one for extraction of gDNA and the other for extraction of RNA and conversion to cDNA. However, as absence of sequencing of a clonal productive rearranged heavy chain immunoglobulin gene from gDNA occurs only in a low proportion of cases (about 10%), directly extracting gDNA from the first part and cDNA from the second part would be useless in many cases.

Therefore, in a preferred embodiment of the method according to the invention, step a) comprises extracting gDNA from a first part of the biological sample and freezing the second part of the biological sample. In this embodiment, gDNA is extracted for analysis from a first part of the biological sample, and the second part of the biological sample is maintained in frozen state for possible further extraction of RNA and conversion to cDNA. In this embodiment, the second part of the biological sample may be frozen in any suitable state maintaining the integrity of RNA comprised in the sample. In particular, some of the extraction steps may or not have been performed before freezing.

For instance, the second part of the biological sample may be frozen as a dry cells' pellet or as a cell lysate in a solution comprising a chaotropic agent.

### Step b)

In step b), rearranged immunoglobulin heavy chain genes are amplified from gDNA and/or cDNA by multiplex polymerase chain reaction (PCR) using primers from a kit according to the invention for determination of the CLL mutational status, as described above.

An appropriate kit is selected depending on the sequencing technique (NGS or Sanger) used in step c).

### Primers to be Used for Further NGS Sequencing

If NGS is used as sequencing technique in step c), then rearranged immunoglobulin heavy chain genes may be amplified from gDNA by multiplex polymerase chain reaction (PCR) with the following primers:
Forward primers comprising respectively the target sequences SEQ ID NO:1 to SEQ ID NO:23, and a reverse primer comprising SEQ ID NO:30; or
Preferably, forward primers consisting respectively, from 5′ to 3′, of a first adapter sequence (preferably of structure 5′-Forward flow cell binding adapter-Index i5-Forward sequencing primer site-3′, wherein the Forward flow cell binding adapter is of sequence SEQ ID NO:34, the Forward sequencing primer site is of sequence SEQ ID NO:35, and the Index i5 is selected from one of the sequences SEQ ID NO:36 to 53 of Table 7 above) fused to one of the target sequences SEQ ID NO:1 to SEQ ID NO:23, and the reverse primer consisting, from 5′ to 3′, of a second adapter sequence (preferably of structure 5′-Reverse flow cell binding adapter-Index i7—Reverse sequencing primer site-3′, wherein the Reverse flow cell binding adapter is of sequence SEQ ID NO:54, the Reverse sequencing primer site is of sequence SEQ ID NO:55, and the Index i7 is selected from one of the sequences SEQ ID NO:56 to 75 of Table 8 above) fused to SEQ ID NO:30.

If NGS is used as sequencing technique in step c), then rearranged immunoglobulin heavy chain genes may be amplified from cDNA by multiplex polymerase chain reaction (PCR) with the following primers:
Forward primers comprising respectively the target sequences SEQ ID NO:24 to SEQ ID NO:29 (and optionally SEQ ID NO: 133), and reverse primer comprising respectively the target sequences SEQ ID NO:31 to SEQ ID NO:33 (and optionally SEQ ID NO: 134); or
Preferably, forward primers consisting respectively, from 5′ to 3′, of a first adapter sequence (preferably of structure 5′-Forward flow cell binding adapter-Index i5-Forward sequencing primer site-3′, wherein the Forward flow cell binding adapter is of sequence SEQ ID NO:34, the Forward sequencing primer site is of sequence SEQ ID NO:35, and the Index i5 is selected from one of the sequences SEQ ID NO:36 to 53 of Table 7 above) fused to one of the target sequences SEQ ID NO:24 to SEQ ID NO:29 (and optionally SEQ ID NO: 133), and reverse primers consisting, from 5′ to 3′, of a second adapter sequence (preferably of structure 5′-Reverse flow cell binding adapter-Index i7—Reverse sequencing primer site-3′, wherein the Reverse flow cell binding adapter is of sequence SEQ ID NO:54, the Reverse sequencing primer site is of sequence SEQ ID NO:55, and the Index i7 is selected from one of the sequences SEQ ID NO:56 to 75 of Table 8 above) fused to one of the target sequences SEQ ID NO:31 to SEQ ID NO:33 (and optionally SEQ ID NO: 134).

In routine determination of CLL mutational status, the reverse primer comprising SEQ ID NO: 134 (or consisting, from 5′ to 3′, of a second adapter sequence (preferably of structure 5′-Reverse flow cell binding adapter-Index i7—Reverse sequencing primer site-3′, wherein the Reverse flow cell binding adapter is of sequence SEQ ID NO:54, the Reverse sequencing primer site is of sequence SEQ ID NO:55, and the Index i7 is selected from one of the sequences SEQ ID NO:56 to 75 of Table 8 above) fused to SEQ ID NO: 134) may not be used, as two separated amplification reactions are then preferred (one using reverse primers comprising respectively SEQ ID NO:31 and SEQ ID NO:33 as reverse primers and the other using reverse primers comprising respectively SEQ ID NO:32 and SEQ ID NO:134 as reverse primers).

In case no clonal productive rearrangement is detected (neither when using gDNA nor when using cDNA), then two new amplifications further using reverse primers comprising respectively SEQ ID NO:31 and SEQ ID NO:33 as reverse primers, on the one hand, and reverse primers comprising respectively SEQ ID NO:32 and SEQ ID NO:134 as reverse primers, on the other hand, may then be performed using cDNA.

### Primers to be Used for Further Sanger Sequencing

If Sanger sequencing is used as sequencing technique in step c), then rearranged immunoglobulin heavy chain genes may be amplified from gDNA by multiplex polymerase chain reaction (PCR) with the following primers:
Forward primers comprising respectively the target sequences SEQ ID NO:24 to SEQ ID NO:29 (and optionally SEQ ID NO: 133), and a reverse primer comprising SEQ ID NO:30; or
Preferably, forward primers consisting respectively of the target sequences SEQ ID NO:24 to SEQ ID NO:29 (and optionally SEQ ID NO: 133), and the reverse primer consisting of SEQ ID NO:30.

If Sanger sequencing is used as sequencing technique in step c), then rearranged immunoglobulin heavy chain genes may be amplified from cDNA by multiplex polymerase chain reaction (PCR) with the following primers:
Forward primers comprising respectively the target sequences SEQ ID NO:24 to SEQ ID NO:29 (and optionally SEQ ID NO: 133), and reverse primer comprising respectively the target sequences SEQ ID NO:31 to SEQ ID NO:33 (and optionally SEQ ID NO: 134); or
Preferably, forward primers consisting respectively of SEQ ID NO:24 to SEQ ID NO:29 (and optionally SEQ ID NO: 133), and reverse primers consisting respectively of the target sequences SEQ ID NO:31 to SEQ ID NO:33 (and optionally SEQ ID NO: 134).

In routine determination of CLL mutational status, the reverse primer comprising (or preferably consisting of) SEQ ID NO:134 may not be used, as two separated amplification reactions are then preferred (one using reverse primers comprising or preferably consisting of respectively SEQ ID NO:31 and SEQ ID NO:33 as reverse primers and the other using reverse primers comprising or preferably consisting of respectively SEQ ID NO:32 and SEQ ID NO:134 as reverse primers).

In case no clonal productive rearrangement is detected (neither when using gDNA nor when using cDNA), then two new amplifications further using reverse primers comprising respectively SEQ ID NO:31 and SEQ ID NO:33 as reverse primers, on the one hand, and reverse primers comprising respectively SEQ ID NO:32 and SEQ ID NO:134 as reverse primers, on the other hand, may then be performed using cDNA.

Preferred Amplification Based on gDNA

Preferably, step b) comprises amplifying rearranged immunoglobulin heavy chain genes from gDNA by multiplex polymerase chain reaction (PCR) using primers from a kit according to the invention for determination of the CLL mutational status from gDNA, as described above.

### Amplification Conditions

As the methods according to the invention are intended for determination of CLL mutational status, which is based on detection of the presence of somatic hypermutations in the IGHV gene compared to the closest germline IGHV gene, amplification should preferably be performed with any “high fidelity DNA polymerase”, as defined and described in the section relating to kits.

In addition, the inventors found that amplification is optimal when using a concentration of Mg²⁺ ions of about 3.5 mM and an extension step temperature of about 68° C. As a result, step b) is preferably performed using a concentration of Mg²⁺ ions between 3 and 4 mM (preferably between 3.3 and 3.7 mM, between 3.4 and 3.6 mM, such as about 3.5 mM) and/or using an extension step temperature between 65 and 70° C. (preferably between 66° C. and 70° C., between 67 and 69° C., between 67.5 and 68.5° C., such as about 68° C.).

### Optional Further Amplification of Internal Control

Step b) may further comprise amplifying rearranged immunoglobulin heavy chain genes from an internal control comprising nucleic acid molecules (preferably plasmids) comprising respectively the sequences SEQ ID NO:76 to SEQ ID NO:122.

Optional Simultaneous Amplification of Samples from Distinct Patients

Step b) may be performed in parallel for several distinct gDNA or cDNA (preferably gDNA) samples from distinct patients.

This embodiment is particularly useful when NGS sequencing is used, as this sequencing technique is particularly suitable for simultaneous sequencing of many samples.

### Step c)

After amplification of the CLL clonal IGH-VDJ rearrangements, PCR products may be purified and are thereafter sequenced.

In step c), amplified rearranged heavy chain immunoglobulin genes are sequenced using either Sanger or NGS sequencing depending on the composition of the kit and a clonal productive rearranged heavy chain immunoglobulin gene is identified.

### Ngs Sequencing (See FIG. 3)

“Next generation sequencing” or “New generation sequencing” or “NGS” refers to high throughput sequencing technologies in which clonally amplified DNA templates, or single DNA molecules, are sequenced in a massively parallel fashion in a flow cell.

NGS sequencing typically contains the following substeps:
i) Preparing a library;
ii) Sequencing; and
iii) Bioinformatics analysis of short reads.

In substep i), sequencing libraries are typically created by fragmenting DNA and adding specialized adapters to both ends. An “adapter” is a short double-stranded nucleotide sequence that serves for the sequencing step. Adapters comprise platform-specific sequences for fragment recognition by the sequencer, including a “flow cell binding sequence” for binding to the flow cells of the sequencing instrument, and a “sequencing primer site” for binding of sequencing primers. Each NGS instrument provider uses a specific set of sequences for this purpose. An adapter preferably further comprises an “index” or “barcode” (used interchangeable as synonymous), i.e. a short nucleotide sequence that serves to distinguish the DNA fragments from the biological sample to which they are added from the DNA fragments from another biological sample (to which adapters with distinct index(es) will have been added). This allows for multiple samples to be mixed or pooled together and sequenced at the same time. For example, barcodes 1-20 can be used to individually label 20 samples and then analyze them in a single sequencing run. This approach, called “pooling” or “multiplexing”, saves time and money during sequencing experiments and controls for workflow variation, as pooled samples are processed together. Preferably indexes are used on both 5′ and 3′ primers: such a double indexing increases the ability to analyze simultaneously larger number of samples. In this case, pooling may be performed at the end of library preparation substep i). Subsequent bioinformatics analysis allows to sort out sequences and attribute them to a given sample without ambiguity. Alternatively, the adapters may not contain indexes. In this case, either pooling is not possible, or indexes may be added to the DNA fragments later in the process before sequencing substep ii).

In the context of the methods of the invention, substep i) of preparing a library will not be necessary when the forward and reverse primers used in step b) already contained adapter sequences in 5′ (preferred embodiment). In this case, step c) directly starts by substep ii) of sequencing. Alternatively, if the forward and reverse primers used in step b) do not contain adapter sequences in 5′ (less preferred embodiment), adapter sequences may be ligated to the amplified products of step b).

Before substep ii), multiple libraries can be pooled together and sequenced in the same run—a process known as multiplexing, when adapters contain an index sequence. These indexes are used to distinguish between the libraries during data analysis. In the context of the methods of the invention, when the forward and reverse primers used in step b) already contained adapter sequences in 5′ (preferred embodiment), primers with distinct adapter pairs (preferably each comprising an index) will then be used for distinct patients. Alternatively, when the forward and reverse primers used in step b) do not contain adapter sequences in 5′ (less preferred embodiment), distinct adapter pairs are ligated to amplification products of distinct patients.

In substep ii), sequencing of the library is performed. While various NGS sequencing techniques are known in the art, amplified rearranged heavy chain immunoglobulin genes have a size of about 350-450 bp. As a result, Illumina MiSeq platform with bidirectional 300 bp-long reads is preferred as it is the most suitable in the context of the invention.

In substep iii), sequences are analyzed on dedicated bioinformatics platforms, such as ARResT/interrogate (Bystry V, Reigl T, Krejci A, et al. ARResT/Interrogate: an interactive immunoprofiler for IG/TR NGS data. Bioinformatics. 2017; 33(3):435-437) or Vidjil (Duez M, Giraud M, Herbert R, et al. Vidjil: A Web Platform for Analysis of High-Throughput Repertoire Sequencing. PLoS One. 2016; 11(11):e0166126), although other tools are also possible which determine the CLL “clonotype(s)”, i.e. the sequence(s) of the clonal rearranged heavy chain immunoglobulin gene(s) present in each patient's sample. Indeed, a biological sample from a CLL sample may potentially contain several clonal rearranged heavy chain immunoglobulin gene(s), most of the time because both alleles of heavy chain immunoglobulin genes of CLL cells have been rearranged (generally one is a non-productive rearrangement, i.e. does not encode a functional immunoglobulin heavy chain, for instance because of a stop codon due to a substitution or a change of reading frame due to insertions/deletions). When two distinct clonotypes are found, one productive and the other non-productive, only the sequence of the clonal productive rearranged heavy chain immunoglobulin gene is further analyzed in step d).

### Sanger Sequencing (see FIG. 4)

Sanger sequencing is performed using reagents and methods well-known in the art.

Briefly, Sanger sequencing is a method of DNA sequencing based on the selective incorporation of chain-terminating dideoxynucleotides by DNA polymerase during in vitro DNA replication. This conventional chain-termination method requires a single-stranded DNA template, a DNA primer, a DNA polymerase, normal deoxynucleotide triphosphates (dNTPs), and modified di-deoxynucleotide triphosphates (ddNTPs), the latter of which terminate DNA strand elongation. ddNTPs are usually fluorescently labeled, each distinct ddNTP being labeled by a distinct fluorescent label, thus permitting easy sequence reading.

Sanger sequencing may be performed using any suitable apparatus, including sequencers from Applied Biosystems (ABI 3730 for instance).

Sanger sequencing is performed in both directions to ensure optimal quality. After alignment of both strands, the resulting consensus sequence is used for further analysis.

When only one clonal rearranged heavy chain immunoglobulin gene is present in the amplified products, PCR products may be sequenced directly, without previous cloning.

In rare instances, if the PCR products contain two clonal rearranged heavy chain immunoglobulin genes (one productive and the other non-productive), cloning of PCR products may be necessary to obtain sequences of the two clonal rearranged heavy chain immunoglobulin genes and identify the clonal productive rearranged heavy chain immunoglobulin gene. In this case, only the sequence of the clonal productive rearranged heavy chain immunoglobulin gene is further analyzed in step d). Alternatively, switching to cDNA template might be helpful as the unproductive rearranged heavy chain immunoglobulin gene tend to be much less transcribed than the productive ones due to the mechanism of nonsense-mediated RNA decay (Delpy L et al. Proc Natl Acad Sci USA. 2004 May 11; 101(19):7375-80).

### Optional Steps c1) to c3)

In some cases, no clonal productive rearranged heavy chain immunoglobulin gene will be identified in step c). This may happen when somatic hypermutations (SHM) are present in the L2 IGHV region or in the IGHJ region, most of the time when SHM are present in the IGHJ region, in the sequence targeted by the reverse primer comprising the sequence SEQ ID NO:30.

In this case, in order to still be able to determine the CLL mutational status of the patient, the method further comprises between step c) and step d) the additional steps of:
c1) extracting RNA from the second part of the biological sample and converting it to complementary DNA (cDNA) or providing cDNA previously obtained from the second part of the biological sample,
c2) amplifying rearranged immunoglobulin heavy chain genes from cDNA by multiplex polymerase chain reaction (PCR) with the following primers:
   i) Forward primers comprising respectively the target sequences SEQ ID NO:24 to SEQ ID NO:29, (and optionally SEQ ID NO: 133) and
   ii) Reverse primers comprising respectively the target sequences SEQ ID NO:31 to SEQ ID NO:33, (and optionally SEQ ID NO: 134) and
c3) sequencing amplified rearranged heavy chain immunoglobulin genes using either Sanger or NGS sequencing and identifying a clonal productive rearranged heavy chain immunoglobulin gene,
   wherein step d) is performed on the clonal productive rearranged heavy chain immunoglobulin gene identified in step c3).

When NGS sequencing is used in step c3), amplification of rearranged immunoglobulin heavy chain genes from cDNA by multiplex polymerase chain reaction (PCR) is step c2) is preferably with the following primers:
i) Forward primers consisting respectively, from 5′ to 3′, of a first adapter sequence fused to one of the target sequences SEQ ID NO:24 to SEQ ID NO:29, (and optionally SEQ ID NO: 133) and
ii) Reverse primers consisting respectively, from 5′ to 3′, of a second adapter sequence fused to one of the target sequences SEQ ID NO:31 to SEQ ID NO:33 (and optionally SEQ ID NO: 134).

When Sanger sequencing is used in step c3), amplification of rearranged immunoglobulin heavy chain genes from cDNA by multiplex polymerase chain reaction (PCR) is step c2) is preferably with the following primers:
i) Forward primers consisting respectively of the target sequences SEQ ID NO:24 to SEQ ID NO:29, (and optionally SEQ ID NO: 133) and
ii) Reverse primers consisting respectively of the target sequences SEQ ID NO:31 to SEQ ID NO:33 (and optionally SEQ ID NO: 134).

### Step d)

In step d), the clonal productive rearranged heavy chain immunoglobulin gene identified in step c) or c3) is aligned to germline immunoglobulin IGH variable region genes, the percentage of identity between the IGHV gene of the identified clonal productive rearranged heavy chain immunoglobulin gene and its closest germline IGHV gene is determined.

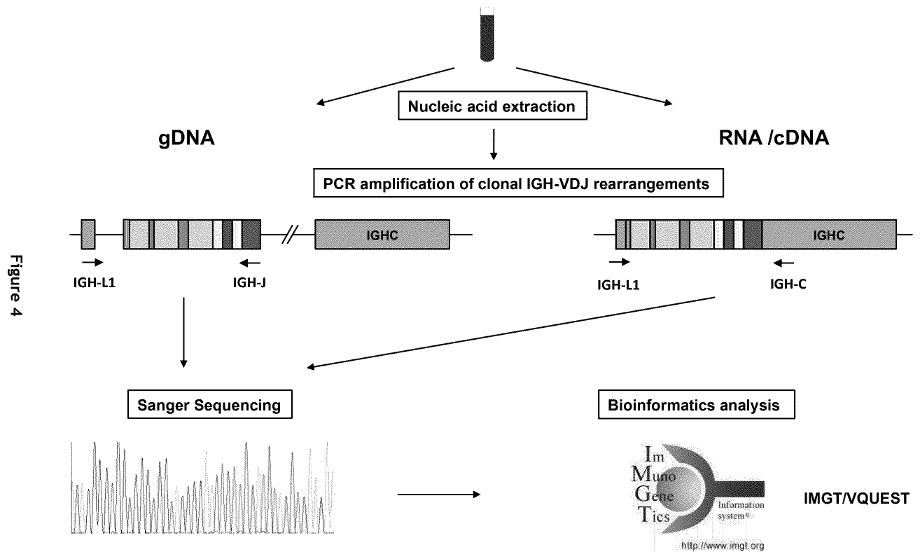

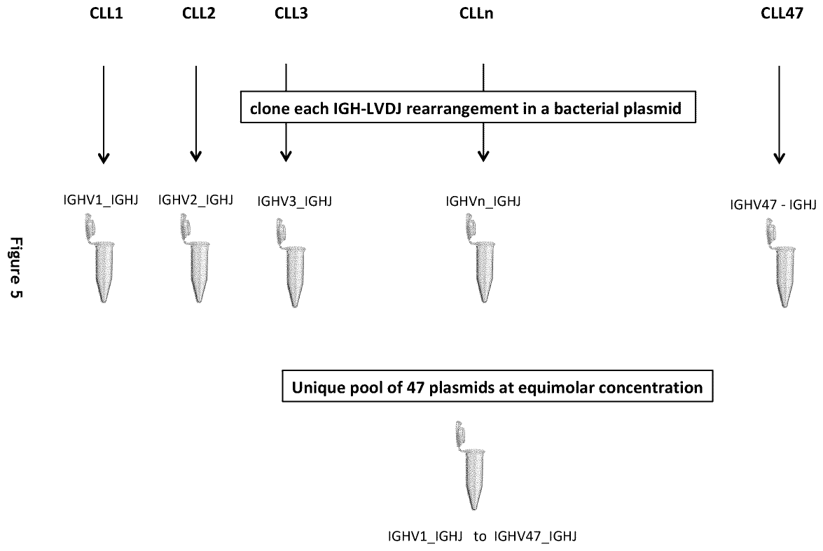

The recommended way to do it is to use the IMGT/V-QUEST software (Lefranc M P, Giudicelli V, Ginestoux C, et al. IMGT, the international ImMunoGeneTics information system. Nucleic Acids Res. 2009; 37(Database issue):D1006-12), which automatically determines percentage of identity of the IGHV part of the identified clonal productive rearranged heavy chain immunoglobulin gene compared to its closest germline counterpart (see FIG. 2).

### Step e)

In step e), the mutational status of the CLL patient is determined as follows:
Unmutated (U-CLL) if the percentage of identity between the IGHV gene of the identified clonal productive rearranged heavy chain immunoglobulin gene and its closest germline immunoglobulin IGHV gene is equal or higher than 98% (corresponding to 2% or less mutations), and
mutated (M-CLL) if the percentage of identity between the IGHV gene of the identified clonal productive rearranged heavy chain immunoglobulin gene and its closest germline immunoglobulin IGHV gene is below 98% (corresponding to more than 2% mutations).

The following examples merely intend to illustrate the present invention.

### EXAMPLES

### Example 1: Design of Suitable Sets of Primer for Determination of CLL Mutational Status from gDNA and cDNA by NGS or Sanger Sequencing

### Material & Methods

### 1) Obtention of Germline IGH Sequences

Germline sequences of all functional human IGHV, IGHJ and IGHC genes and their available alleles were retrieved from the IMGT database (http://www.imgt.org/genedb/). These included 192 IGHV sequences, 13 IGHJ sequences and 66 IGHC sequences. More specifically, for IGHV genes, only the upstream region corresponding to the Leader part 1 (L1)—intron—Leader part 2 (L2) region was obtained. This corresponded to a mean 142 nucleotide-long sequence. In some instances, a few upstream extra nucleotides were also incorporated.

### 2) Location of Primers

The locations of the primers were chosen in order to obtain the most informative sequences for optimal IGH mutational status assessment e.g. containing the entire V region. They also depended on constraints linked to the amplicon length capacity of either Sanger or NGS technologies (longer sequences possible with Sanger sequencing). The IGHV-L1 primers were positioned at the very beginning of the L1 sequence or a few nucleotides downstream. For IGHV-L2 primers, a 41 bp region corresponding to the L2 coding sequence+30 upstream nucleotides was selected for primer positioning. IGHJ primers were located in the 3′ part of the gene, while the 5′ part was chosen for IGHC genes.

### 3) General Features

Sequences from each available allele for each gene were aligned and when nucleotide variations occurred a consensus sequence determined. Depending on the target and the sequencing methodology (NGS or Sanger), either gene specific primers or consensus primers for a set of genes belonging to the same subgroup were chosen. Depending on the gene characteristics (such as the GC content) a 18-25 long stretch of nucleotides was selected for primer positioning.

### 4) in Silico Design

Several tools were used for in silico primer design and evaluation, such as Primer 3 (https://primer3.org), MFE Primer 2.0 (http://biocompute.bmi.ac.cn/CZlab/MFEprimer-2.0), OLIGO 7 (www.oligo.net) and CLUSTAL (https://www.ebi.ac.uk/Tools/msa/clustalo).

Parameters taken in consideration included primer length, GC content, annealing temperature (57-63° C.), priming efficiency, probability to form homodimers and hairpins, ability to be combined for multiplexing.

### 6) Initial Testing

Several levels of testing were performed.

### Structure of Primers

For NGS-based assays primers were first evaluated in an unmodified form, e.g. without the adapter sequences, thereby focusing on their target binding specificity. The exception was the 3′ primers which incorporated a fluorescent tag (FAM) allowing to analyse the PCR products by capillary electrophoresis (Genescan). In a second phase, full size primers with adapter sequences were re-evaluated with optimal PCR conditions as defined during the first test phase. Here again, the 3′primers had a fluorescent tag for Genescan analysis of the resulting PCR products. For Sanger-based assay, only the first phase testing was performed as no further modification was needed.

### Number of Primers

Tests were first performed using pairs of individual primers, and then combining them in a multiplex PCR.

### PCR Conditions

Various parameters were evaluated during the PCR tests including annealing temperature, magnesium concentrations, primer concentration and relative amount of each of them, number of cycles, type of Taq polymerase (standard vs high-fidelity).

### Targets

Initial testing was performed on both polyclonal (blood from healthy individuals, reactive lymph node biopsies) and monoclonal samples. The latter consisted in CLL cases for which the IGH-VDJ sequences had previously been determined using FR1 Biomed-2 primers (van Dongen et al. Leukemia. 2003; 17(12):2257-317). For initial testing only one representative sample from each of the 7 IGHV subgroups was used, but latter on evaluation was performed on an extended series of cases including all functional IGHV and IGHJ genes (respectively 48-55 and 6). In addition, the 47 plasmid mix was used to further evaluate and optimize the primer mixes and PCR conditions.

### Results

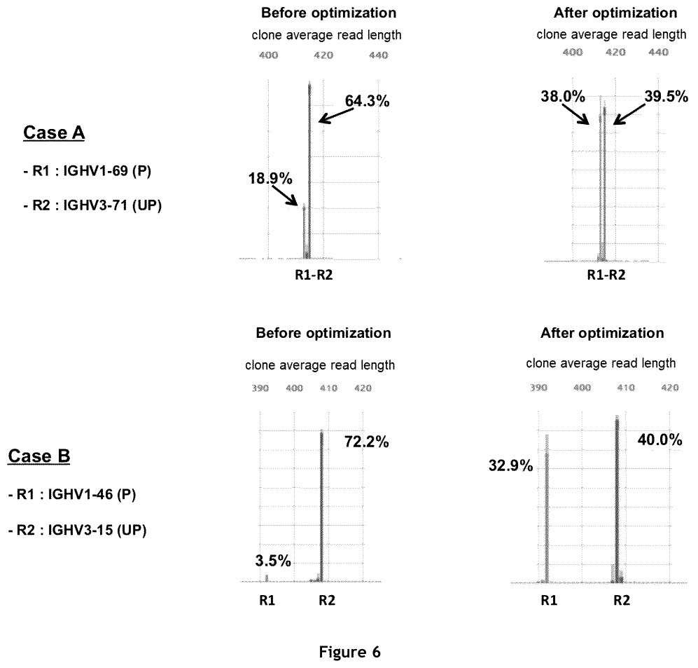

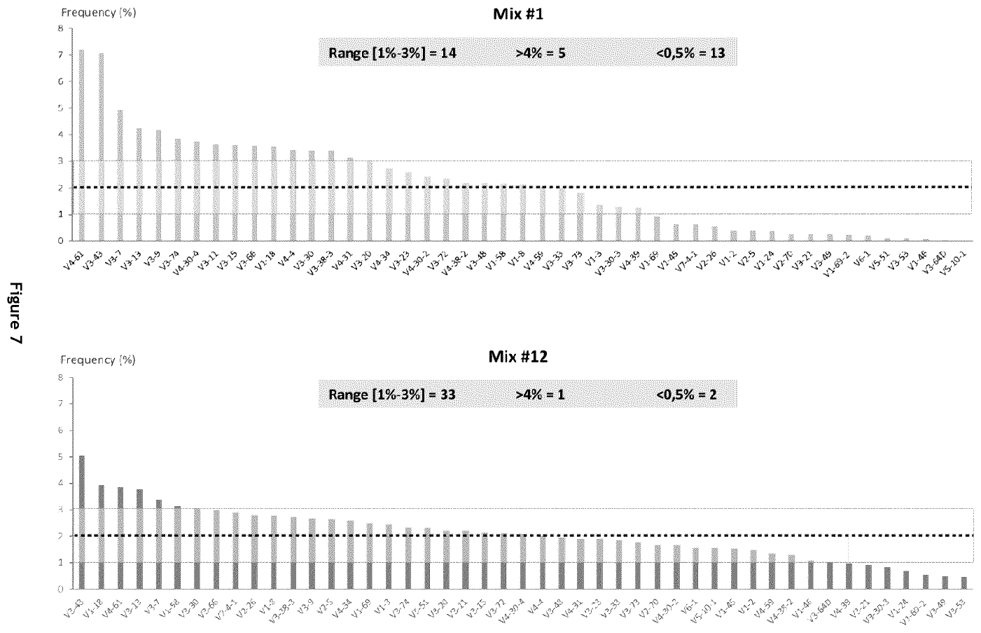

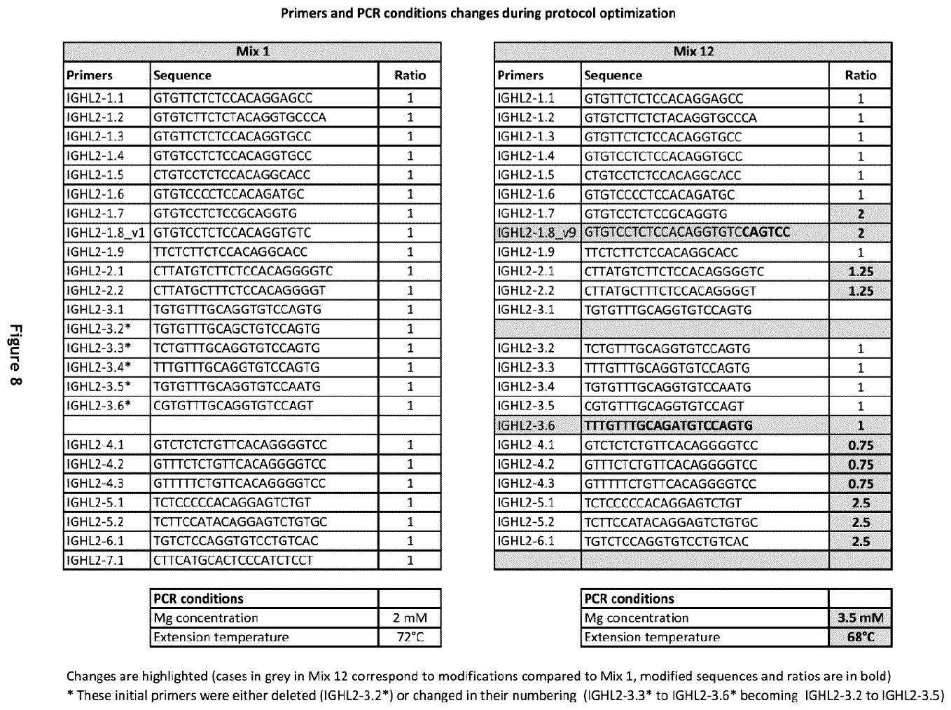

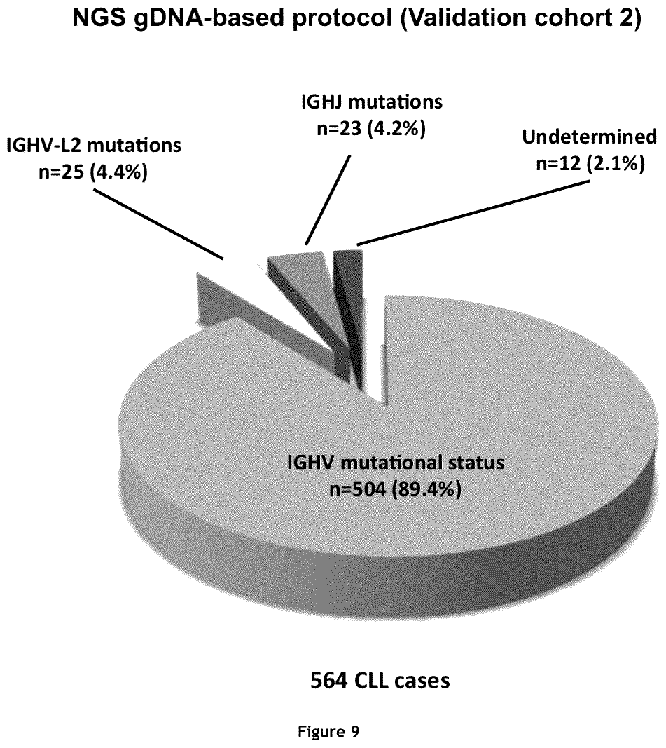

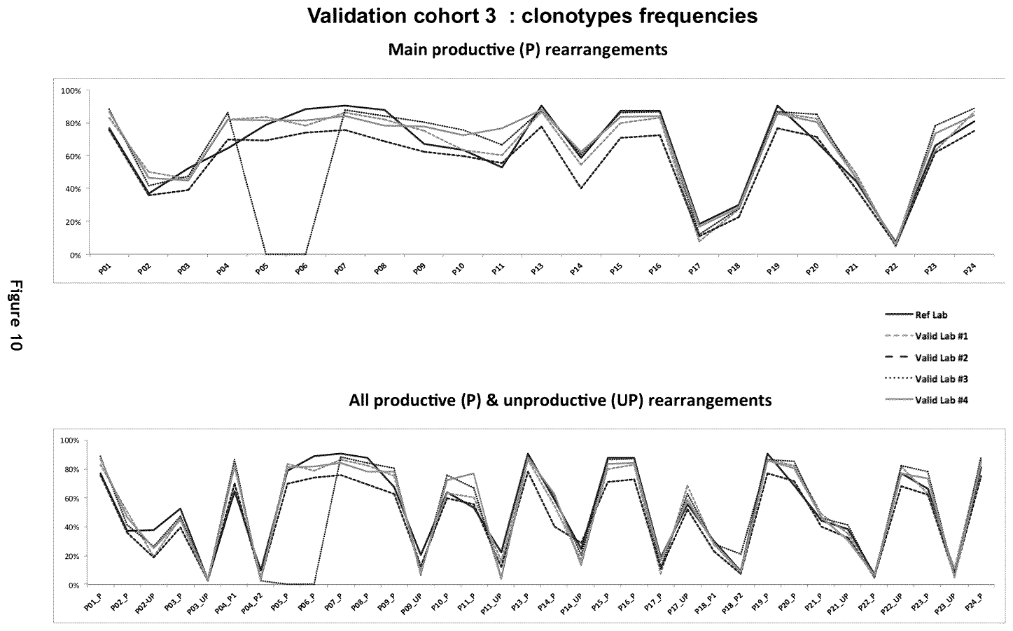

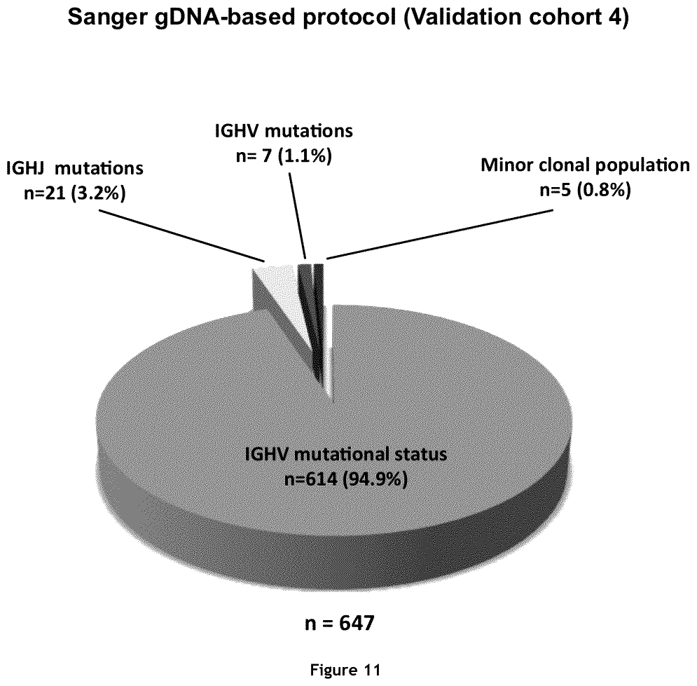

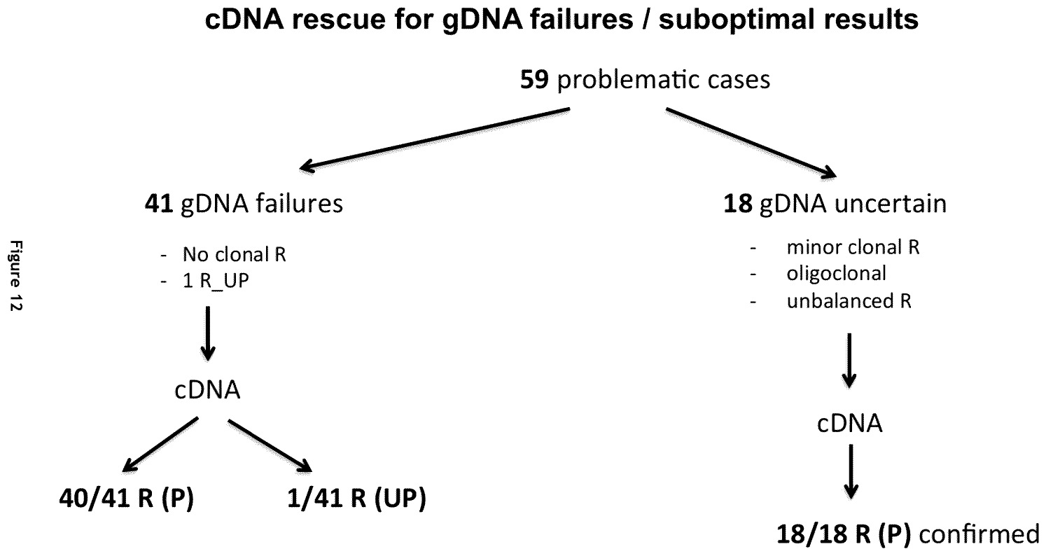

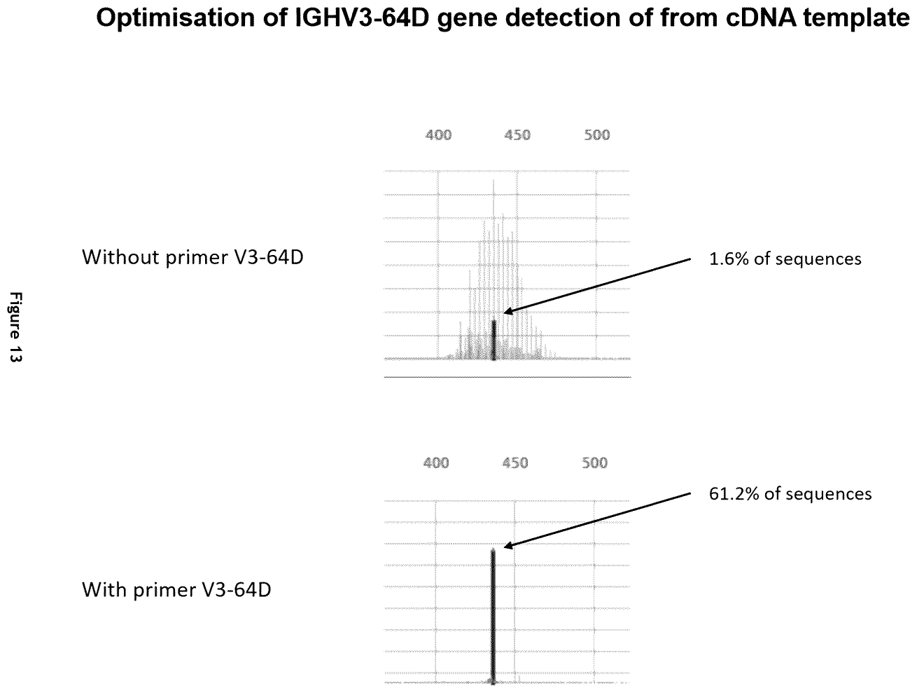

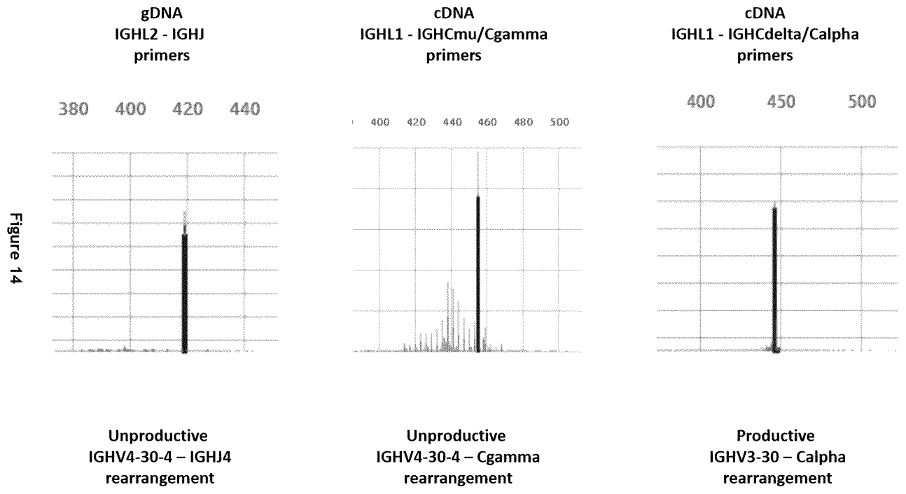

Once the initial PCR conditions were determined, the assays were evaluated on a larger series of CLL cases collected from routine practice. As the NGS methodology provides quantitative values for each rearrangement clonotype present in a given sample, it became clear that the protocols designed initially could benefit from further optimization. This was particularly true for the NGS-gDNA protocol employing the largest number of IGHV primers (n=24 initially) and for which amplification biases could clearly be seen. For some cases with biallelic rearrangements this resulted in important imbalances between the clonotype frequencies from each allele (FIG. 6).

Further appreciation of this bias was evaluated using the 47 plasmid mix (P01 to P47, SEQ ID NO:76 to 122), which contains an equimolar concentration of IGH-VDJ rearrangements accounting for all but one of the 48 human functional IGHV genes (the omitted one being very rarely used in CLL). Therefore, in case of similar amplification efficiency from each IGHV-L2 primer, the frequency of each rearrangement within the 47 plasmid mix should be roughly 2%. As can be seen from FIG. 7 (top), this was not the case when using the first designed mix of primers (Mix #1) as some rearrangements were over-amplified while other were clearly under-amplified.

Further changes were thus implemented including varying PCR conditions (magnesium concentration, extension step temperature) and primers relative concentration as well as primer sequence in some instances.

In particular:
11 versions of the IGHL2-1.8 primer (recognizing the IGHV1-69 gene) were tested (Table 10):

TABLE 10
11 tested versions of the IGHL2-1.8 primer. The version used in Mix #1 of
FIG. 7 (top) is IGHL2-1.8_v1, while the version used in final Mix #12 of
FIG. 7 (bottom) is IGHL2-1.8_v9 (in bold).

| | | SEQ ID NO: |
|---|---|---|
| Primer | Sequence | (comments) |
| IGHL2-1.8_v1 | GTGTCCTCTCCACAGGTGTC | SEQ ID NO: 123 (Mix #1) |
| IGHL2-1.8_v2 | GTGTCCTCTCCACAGGTGTCC | SEQ ID NO: 124 |
| IGHL2-1.8_v3 | GTCCTCTCCACAGGTGTCCA | SEQ ID NO: 125 |
| IGHL2-1.8_v4 | CTGTGTCCTCTCCACAGGTGTC | SEQ ID NO: 126 |
| IGHL2-1.8_v5 | GTTTCCTCTCCACAGGTGTC | SEQ ID NO: 127 |
| IGHL2-1.8_v6 | GGGTCCTCTCCACAGGTGTC | SEQ ID NO: 128 |
| IGHL2-1.8_v7 | GTGTCCTCTCCACAGGTGTCCA | SEQ ID NO: 129 |
| IGHL2-1.8_v8 | GTGTCCTGTCCACAGGTGTC | SEQ ID NO: 130 |
| IGHL2-1.8_v9 | GTGTCCTCTCCACAGGTGTCCAGTCC | SEQ ID NO: 8 (Mix #12) |
| IGHL2-1.8_v10 | GTGTCCTGTCCACAGGTGTCCAGTCC | SEQ ID NO: 131 |
| IGHL2-1.8_v11 | GTGTCCTSTCCACAGGTGTCCAGTCC | SEQ ID NO: 132 |

Two primers (initial primer IGHL2-3.2* and IGHL2-7.1) of the first designed mix of primers (Mix #1) were found to be unnecessary while a new primer (IGHL2-3.6) was added to the final mix (Mix #12).
   FIG. 8 shows differences between the composition of Mix #1 and Mix #12, and also between PCR conditions used when assessing Mix #1 and Mix #12 on the 47 plasmid mix (P01 to P47, SEQ ID NO:76 to 122).

A total of 12 mixes were tested, resulting for the last one (Mix #12) in a substantial improvement of results. This can be seen in FIG. 7: when considering the theoretical expected frequency for each plasmid IGH-VDJ rearrangement clonotype, 14 were found in a 2%±1% range in the Mix #1 but 33 in Mix #12. The number of extreme outliers was also substantially reduced in Mix #12 as compared to Mix #1 with 1 vs 5 plasmid IGH-VDJ rearrangement being detected with a frequency>4%, and 2 vs 13 with a frequency<0.5%. The improvement was also clearly seen for cases with biallelic rearrangements. FIG. 6 shows two examples where modification of primer sequence and primer ratio had a clear impact on the balance of the 2 rearrangements clonotype frequencies.

Following design and validation procedures, the final number of primers was the following:

TABLE 11
Final number of primers.

| Assay | 5′ primers | 3′ primers |
|---|---|---|
| Sanger gDNA | IGHV-L1: 6 | IGHJ: 1 |
| Sanger cDNA | IGHV-L1: 6 | IGHC: 3 |
| NGS gDNA | IGHV-L2: 23 | IGHJ: 1 |
| NGS cDNA | IGHV-L1: 6 | IGHC: 3 |

### Example 2: Validation of the Developed Sets of Primer for Determination of CLL Mutational Status from gDNA and cDNA by NGS or Sanger Sequencing

Validation of Primer Sets for NGS Methodology from gDNA

### Validation on an Extended Panel of CLL Cases (Validation Cohort 1)

In order to validate our method and to check whether all IGHV and IGHJ genes could be amplified with our primer combinations, a series of 95 CLL (74.7% monoallelic and 25.3% biallelic) whose IGH-VDJ rearrangement sequences had been previously obtained with Sanger sequencing were tested. In total they accounted for 126 IGH-VDJ sequences, including mutated (34.9%) as well as unmutated (65.1%), productive (80.2%) and unproductive (19.8%) rearrangements. As can be seen from Table 12, the selected cases used all possible IGHJ and IGHV genes, excepted for 4 IGHV genes rarely used in CLL (less than 0.2% in a cohort of ≈30 000 sequences, as reported by Agathangelidis et al., Blood 2021: 137(10):1365-1376. doi:10.1182/blood. 2020007039) and for which no case was available. However, cases expressing these IGHV genes were later detected with this methodology during laboratory routine practice (excepted for IGHV3-NL1).

TABLE 12
Validation cohort 1.

| IGHV | Nb | Mono- | Additional | Bi- | | | IGHV % | | | | | | | | |
|---|---|---|---|---|---|---|---|---|---|---|---|---|---|---|---|
| Gene | R | allelic | R | allelic | M | UM | identity range | P | UP | IGHJ1 | IGHJ2 | IGHJ3 | IGHJ4 | IGHJ5 | IGHJ6 |
| V1-2 | 6 | 5 | | 1 | 1 | 5 | 92-100 | 6 | | | | | 2 | 1 | 3 |
| V1-3 | 4 | 3 | | 1 | 1 | 3 | 92.4-100 | 3 | 1 | | | | 2 | 1 | 1 |
| V1-8 | 2 | 1 | 1 | | 2 | | 94.4-95.8 | 2 | | 1 | | | 1 | | |
| V1-18 | 2 | 1 | | 1 | 1 | 1 | 95.5-99 | 2 | | | | | 1 | 1 | |
| V1-24 | 1 | 1 | | | | 1 | 100 | 1 | | | | | | 1 | |
| V1-45 | 1 | 1 | | | | 1 | 100 | 1 | | | | | | | 1 |
| V1-46 | 1 | | | 1 | 1 | | 91.6 | | 1 | | | | 1 | | |
| V1-58 | 1 | 1 | | | 1 | | 97.2 | 1 | | | | | | | 1 |
| V1-69/ | 16 | 6 | 3 | 7 | 1 | 15 | 88.2-100 | 14 | 2 | | 1 | 4 | 5 | 1 | 5 |
| V1-69D | | | | | | | | | | | | | | | |
| V1-69-2 | | | | | | | | | | | | | | | |
| V2-5 | 1 | 1 | | | 1 | | 95.2 | 1 | | | | | 1 | | |
| V2-26 | 1 | 1 | | | 1 | | 95.2 | 1 | | | | 1 | | | |
| V2-70/ | 1 | | | 1 | | 1 | 100 | | 1 | | | | 1 | | |
| V2-70D | | | | | | | | | | | | | | | |
| V3-7 | 1 | 1 | | | | 1 | 100 | 1 | | | | | | | 1 |
| V3-9 | 4 | 2 | | 2 | | 4 | 99.7-100 | 4 | | | | | 2 | | 2 |
| V3-11 | 2 | 1 | 1 | | 1 | 1 | 97.6-99 | 2 | | | | | 2 | | |
| V3-13 | 2 | | | 2 | | 2 | 98.9-100 | 1 | 1 | | | | 1 | 1 | |
| V3-15 | 3 | 2 | | 1 | 1 | 2 | 96.6-100 | 3 | | | 1 | 1 | | | 1 |
| V3-20 | 1 | 1 | | | 1 | | 96.2 | 1 | | | | | | 1 | |
| V3-21 | 7 | 5 | | 2 | 1 | 6 | 94.1-100 | 6 | 1 | | | | 1 | 2 | 4 |
| V3-23/ | 7 | 3 | | 4 | 3 | 4 | 87.5-100 | 5 | 2 | | | | 4 | 2 | 1 |
| V3-23D | | | | | | | | | | | | | | | |
| V3-30/ | 6 | 2 | 1 | 3 | 4 | 2 | 91.3-100 | 3 | 3 | 1 | | | 3 | | 2 |
| V3-30-5 | | | | | | | | | | | | | | | |
| V3-30-3 | 2 | 2 | | | 1 | 1 | 95.5-100 | 2 | | | | | | | 2 |
| V3-33 | 7 | 2 | | 5 | 2 | 5 | 86.8-100 | 4 | 3 | | | | 5 | | 2 |
| V3-43/ | 2 | | 1 | 1 | | 2 | 100 | | 2 | | | | 2 | | |
| V3-43D | | | | | | | | | | | | | | | |
| V3-48 | 6 | 4 | | 2 | 2 | 4 | 91.7-100 | 5 | 1 | | | 1 | 3 | 2 | |
| V3-49 | 1 | | | 1 | | 1 | 100 | | 1 | | | | 1 | | |
| V3-53 | 1 | 1 | | | 1 | | 94.4 | 1 | | | | | | | 1 |
| V3-64/ | 2 | | | 2 | | 2 | 100 | 1 | 1 | | | | 2 | | |
| V3-64D | | | | | | | | | | | | | | | |
| V3-66 | 2 | 2 | | | 1 | 1 | 95.4-100 | 2 | | | | | 1 | 1 | |
| V3-72 | 2 | 1 | | 1 | | 2 | 92.2-100 | 1 | 1 | | | | 2 | | |
| V3-73 | 1 | 1 | | | 1 | | 91.5 | 1 | | | | | | 1 | |
| V3-74 | 5 | 3 | | 2 | 3 | 2 | 94.8-100 | 5 | | | | 2 | 3 | | |
| V3-NL1 | | | | | | | | | | | | | | | |
| V4-4 | 2 | 1 | | 1 | 2 | | 63.5-93.3 | 1 | 1 | | | | 1 | 1 | |
| V4-28 | | | | | | | | | | | | | | | |
| V4-30-2 | | | | | | | | | | | | | | | |
| V4-30-4 | 1 | 1 | | | | 1 | 100 | 1 | | | | | | | 1 |
| V4-31/ | 1 | 1 | | | 1 | | 93.1 | 1 | | | | | | 1 | |
| V4-30-1 | | | | | | | | | | | | | | | |
| V4-34 | 4 | 2 | | 2 | 1 | 3 | 93.3-100 | 3 | 1 | | | 1 | 2 | | 1 |
| V4-38-2 | 2 | 1 | | 1 | 1 | 1 | 97.2-99.7 | 1 | 1 | | | 1 | | | 1 |
| V4-39 | 2 | 2 | | | 1 | 1 | 93.5-100 | 2 | | | 1 | | | | 1 |
| V4-59 | 5 | 2 | | 3 | 2 | 3 | 90.2-100 | 5 | | | | 1 | 2 | 1 | 1 |
| V4-61 | 2 | 2 | | | 1 | 1 | 96.9-100 | 2 | | | | | 1 | | 1 |
| V5-10-1 | 1 | 1 | | | | 1 | 100 | 1 | | | | | | | 1 |
| V5-51 | 2 | 2 | | | 1 | 1 | 94.1-100 | 2 | | | | 1 | 1 | | |
| V6-1 | 2 | 1 | | 1 | 1 | 1 | 93.3-100 | 1 | 1 | | | 1 | 1 | | |
| V7-4-1 | 1 | 1 | | | 1 | | 96.9 | 1 | | | | | | 1 | |
| Total | 126 | 71 | 7 | 48 | 44 | 82 | | 101 | 25 | 2 | 3 | 14 | 54 | 19 | 34 |

Abbreviations: R, rearrangement; M mutated; UM, unmutated; P, productive; UP, unproductive.

Sequences obtained by NGS were identical to the original ones. Of note, additional rearrangements, not detected initially by Sanger, were found in 7 cases.

Altogether these results show that the methodology developed herein for determination of IGHV mutational status from gDNA by NGS is able to detect virtually all combinations of functional IGHV and IGHJ genes within IGH-VDJ rearrangements from CLL cells.

### Validation on “Real Life” Routine Laboratory Activity (Validation Cohort 2)

After a long phase of optimization, the PCR assay using gDNA as template was used for routine practice on 564 cases of CLL obtained during a 3-year period. As can be seen on FIG. 9, the mutational IGHV status could be determined without ambiguity in 504 (89.4%) of them.

For the remaining cases, additional analyses using cDNA-NGS sequencing and/or gDNA-Sanger sequencing with IGHV-L1 primers showed that failures were caused by SHM preventing annealing of either the 5′ IGHV-L2 primers in 25 (4.4%) cases, or the 3′ IGHJ primer in 23 (4.1%) cases. In 12 (2.1%) cases the reasons could not be determined due to the lack of available material (cDNA).

Thus, altogether the rate of success of used methodology is close to 90%. Of note, switching to the cDNA-based protocol for those cases allowed the identification of the CLL clonotype in the vast majority of these cases.

### Multicenter Validation (Validation Cohort 3)

In order to evaluate the robustness of the methodology, an external validation involving 4 laboratories having expertise in IGHV mutational status assessment was undertaken. A series of gDNA from 23 CLL cases was provided along with primer mixes and detailed protocol. All cases had been previously sequenced both by Sanger and NGS, and a total of 34 IGH-VDJ rearrangements had been identified. Twelve cases had a single productive rearrangement, 9 had both a productive and an unproductive rearrangement, and 2 had 2 productive rearrangements (a major one+a minor one). Seven cases had mutated IGHV genes, while 16 had unmutated IGHV genes. All rearrangements were detected by the 4 laboratories excepted for 2 cases which were missed by one laboratory.

Moreover, NGS provides quantitative values for the frequencies of clonotypes (e.g. identical sequences corresponding to a given IGH-VDJ rearrangement) among all sequences obtained from a sample. Remarkably, very similar clonotype frequencies were obtained by all laboratories (see FIG. 10). Of note, amplification biases observed for some biallelic cases appeared to be highly reproducible.

Overall, this multicenter validation test demonstrates the robustness of the protocol.

Validation of Primer Sets for Sanger Sequencing from gDNA (Validation Cohort 4)

Determination of IGHV mutational from gDNA by Sanger sequencing using IGHV-L1 and IGHJ primers was performed on 647 CLL cases (FIG. 11).

A successful result was obtained in 614 (94.9%) cases. In 21 (3.2%) cases failure was due to SHM occurring in the IGHJ gene preventing primer annealing as suggested by the inability to amplify the clonal IGH-VDJ rearrangement using alternative 5′ primers (IGHV-L2, IGHV-FR1). This was confirmed in 9/21 cases with available cDNA and for which the sequence was obtained using IGHV-L1 and IGHC primers. Alternatively in 7 (1.1%) of cases, the failure was due to mutations within the IGHV-L1 target sequence, as shown by the obtention of a clonal rearrangement using an alternative IGHV primer (IGHV-FR1). Finally in 5 (0.8%) cases, only a minor clonal rearrangement was detected, but the polyclonal background rearrangement prevented the possibility to obtain a good quality sequence by Sanger methodology. These cases were in fact monoclonal B-cell lymphocytosis with a minor circulating CLL-like population, but for which the determination of IGHV mutational status is not recommended (Hallek et al., Blood 2018: 131 (25): 2745-2760. https://doi.org/10.1182/blood-2017-09-806398).

Validation of Primer Sets for NGS and/or Sanger Methodology from cDNA

### Validation on an Extended Panel of CLL Cases (Validation Cohort 5)

In order to validate our method and to check whether all IGHV and IGHC genes could be detected from cDNA templates with our primer combinations, we evaluated a series of 90 CLL comprising 101 IGH-VDJ rearrangements which had been previously characterized from gDNA.

As both Sanger and NGS sequencing methods use the same IGHV and IGHC sequence-specific primers, the validation was done in two phases. In the first one, the 3′ IGHC primers were tagged with a fluorescent dye (6-FAM) and the PCR products analyzed by capillary electrophoresis (Genescan) in order to detect the presence of clonal IGH-VDJC rearrangement(s). In the second phase, the PCR were repeated but using primers adapted for sequencing. The PCR products were thereafter sequenced by NGS, and also for a fraction of cases by Sanger technique.

As can be seen from Table 13 below, the selection of cases comprised almost all functional IGHV genes excepted for 5 of them, for which there was no available material but which are rarely used in CLL (in less than 0.5% of cases in a cohort of ≈30 000 sequences, reported by Agathangelidis et al., Blood 2021: 137(10):1365-1376. doi:10.1182/blood.2020007039). All 3 IGHC genes expressed by CLL cells were also detected by our primers. The cohort included IGH-VDJ rearrangements with unmutated (62%) and mutated (38%) IGHV genes, and 71% were monoallelic while 29% were biallelic or biclonal rearrangements.

TABLE 13
Validation cohort 5.

| IGHV | Nb | Mono- | Bi- | | | IGHV % | | | IGHC- | IGHC- | IGHC- |
|---|---|---|---|---|---|---|---|---|---|---|---|
| Gene | R | allelic | allelic* | M | UM | identity range | P | UP | mu | gamma | delta |
| V1-2 | 3 | | 3 | 2 | 1 | 92.7-98.3 | 2 | 1 | 3 | | |
| V1-3 | 1 | 1 | | 1 | | 91.7 | 1 | | 1 | | |
| V1-8 | 3 | 2 | 1 | 3 | | 92.0-97.9 | 3 | | 3 | | |
| V1-18 | 3 | 3 | | 2 | 1 | 87.5-100 | 3 | | 2 | 1 | |
| V1-24 | 1 | 1 | | | 1 | 98.6 | 1 | | 1 | | |
| V1-45 | 1 | 1 | | | 1 | 100 | 1 | | 1 | | |
| V1-46 | 1 | | 1 | 1 | | 89.6 | 1 | | 1 | | |
| V1-58 | 1 | 1 | | | 1 | 100 | 1 | | 1 | | |
| V1-69/ | 7 | 5 | 2 | | 7 | 100 | 7 | | 7 | | |
| V1-69D | | | | | | | | | | | |
| V1-69-2 | | | | | | | | | | | |
| V2-5 | 3 | 3 | | 2 | 1 | 91.4-100 | 3 | | 3 | | |
| V2-26 | 1 | | 1 | | 1 | 100 | 1 | | 1 | | |
| V2-70/ | 1 | 1 | | 1 | | 96.6 | 1 | | 1 | | |
| V2-70D | | | | | | | | | | | |
| V3-7 | 5 | 3 | 2 | 5 | | 89.9-95.5 | 5 | | 3 | 2 | |
| V3-9 | 2 | 1 | 1 | 1 | 1 | 67.1-100 | 1 | 1 | 2 | | |
| V3-11 | 2 | 2 | | 2 | | 92.7-98.6 | 2 | | 2 | | |
| V3-13 | 2 | 1 | 1 | 1 | 1 | 88.4-100 | 1 | 1 | 2 | | |
| V3-15 | 2 | | 2 | 2 | | 92.5-94.2 | 2 | | 2 | | |
| V3-20 | 1 | 1 | | 1 | | 96.9 | 1 | | | 1 | |
| V3-21 | 4 | 4 | | 3 | 1 | 97.2-98.6 | 4 | | 4 | | |
| V3-23/ | 4 | 3 | 1 | 3 | 1 | 92-98.6 | 4 | | 3 | 1 | |
| V3-23D | | | | | | | | | | | |
| V3-30/ | 3 | 2 | 1 | 2 | 1 | 93.1-100 | 2 | 1 | 3 | | |
| V3-30-5 | | | | | | | | | | | |
| V3-30-3 | 3 | 1 | 2 | 2 | 1 | 96.9-100 | 3 | | 3 | | |
| V3-33 | 2 | 2 | | 2 | | 92.4-93.4 | 2 | | 2 | | |
| V3-43/ | 1 | 1 | | 1 | | 93.1 | 1 | | 1 | | |
| V3-43D | | | | | | | | | | | |
| V3-48 | 4 | 3 | 1 | 1 | 3 | 95.8-100 | 4 | | 4 | | |
| V3-49 | 2 | 2 | | 1 | 1 | 96.9-98.6 | 2 | | 2 | | |
| V3-53 | 1 | 1 | | | 1 | 100 | 1 | | 1 | | |
| V3-64/ | | | | | | | | | | | |
| V3-64D | | | | | | | | | | | |
| V3-66 | 2 | 2 | | 2 | | 89.8-93.7 | 2 | | 2 | | |
| V3-72 | 3 | 2 | 1 | 2 | 1 | 92.5-99.0 | 3 | | 3 | | |
| V3-73 | 1 | 1 | | 1 | | 91.5 | 1 | | 1 | | |
| V3-74 | 4 | 3 | 1 | 3 | 1 | 93.1-99.3 | 3 | 1 | 3 | 1 | |
| V3-NL1 | | | | | | | | | | | |
| V4-4 | 3 | 2 | 1 | 3 | | 88.5-91.7 | 3 | | 3 | | |
| V4-28 | | | | | | | | | | | |
| V4-30-2 | | | | | | | | | | | |
| V4-30-4 | 1 | 1 | | | 1 | 100 | 1 | | 1 | | |
| V4-31/ | 1 | 1 | | | 1 | 100 | 1 | | 1 | | |
| V4-30-1 | | | | | | | | | | | |
| V4-34 | 7 | 5 | 2 | 4 | 3 | 93.7-100 | 7 | | 6 | 1 | |
| V4-38-2 | 1 | 1 | | | 1 | 100 | 1 | | 1 | | |
| V4-39 | 1 | 1 | | 1 | | 96.9 | 1 | | 1 | | |
| V4-59 | 2 | 1 | 1 | 2 | | 90.9-92.6 | 2 | | 2 | | |
| V4-61 | 3 | 2 | 1 | 1 | 2 | 96.2-99.7 | 3 | | 3 | | |
| V5-10-1 | 2 | 1 | 1 | 1 | 1 | 94.8-100 | 2 | | 2 | | |
| V5-51 | 2 | 1 | 1 | 1 | 1 | 90.6-100 | 2 | | 1 | | 1 |
| V6-1 | 2 | 2 | | 2 | | 94.6-95.6 | 2 | | 2 | | |
| V7-4-1 | 2 | 1 | 1 | 1 | 1 | 96.9-100 | 1 | 1 | 2 | | |
| Total | 101 | 72 | 29 | 63 | 38 | 67.1-100 | 95 | 6 | 93 | 7 | 1 |

*or biclonal.
Abbreviations: R, rearrangement (number); M mutated; UM, unmutated; P, productive; UP, unproductive.

All sequences obtained from cDNA were identical to those originating from gDNA.

These results show that the methodology developed herein for determination of IGHV mutational status from cDNA by NGS or Sanger sequencing is able to detect virtually all combinations of functional IGHV and IGHC genes within clonal IGH-VDJC rearrangements from CLL cells.

Validation of the cDNA-Based Methodology as a Complementary or Alternative Strategy (FIG. 12)

DNA (gDNA) is often the preferred nucleic acid template used for IGHV mutational status assessment as other genomic investigations such as detection of TP53 gene mutations are often required and performed at the same time for prognostication and treatment choice. However, as shown above, failure to detect and sequence the tumor clonal productive rearrangement(s) was observed with our methodology using Sanger sequencing or NGS in about 5% and 10% of cases respectively. This was due essentially to somatic hypermutations creating mismatches at the primer binding sites. To check whether these could be by-passed using alternative set of primers, we selected 41 cases for which no clonal productive rearrangement could be detected by our gDNA NGS-based method and for which RNA and thus cDNA templates were available. In 40/41 of them a clonal productive rearrangement could be obtained from with our alternative cDNA-based NGS method. In 1/41 cases, only an unproductive clonal rearrangement was detected, similarly to what was observed from gDNA.

In addition, we also selected 18 additional cases for which results using the gDNA NGS-based method were uncertain due the detection of either a single minor clonal rearrangement, or an unbalance between a minor productive rearrangement and a major unproductive one, or more than 2 rearrangements. In all 18 cases, a major productive rearrangement was obtained from the cDNA template by NGS, allowing determination of the IGHV mutational status without ambiguity.

These results show that the cDNA-based approach constitutes a complementary useful strategy to the gDNA-based one, resulting in the ability to detect the tumor clonal productive rearrangement and determine its IGHV mutational status in virtually all CLL cases.

### Multicenter Validation (Validation Cohort 6)

As for gDNA, the robustness of the cDNA NGS-based protocol was assessed through an external validation involving 2 experienced laboratories. A cohort of 24 CLL cases was selected, which had been previously tested both at the gDNA and cDNA levels. On cDNA, 20 cases displayed a single productive rearrangement while 4 harbored 2 rearrangements, productive/unproductive for 3 of them and one having 2 productive rearrangements. In 4 cases the IGH-VDJ rearrangements (3 productive and 1 unproductive) were detected only from IGHCgamma transcripts. The % of identity for the IGHV gene ranged from 87.8% to 100%, with 11 rearrangements being unmutated and 17 mutated. All IGHV subgroups (IGHV1 to IGHV6) were represented excepted IGHV7. The cDNAs from these 24 samples were sent to the 3 external laboratories as well as primer mixes and detailed protocol. All rearrangements were detected by those laboratories with sequences identical to the original ones. These results demonstrate the high reproducibility of this protocol.

### General Conclusion

The combination of assays presented here constitutes a highly efficient method to determine IGHV mutational in CLL. The methodology is adapted to both Sanger and NGS sequencing technologies, with the latter being increasingly used in the majority of laboratories.

The strategy developed here targets gDNA template in first intention, as it is easier to obtain, more stable and allows other genomic investigations often performed in parallel such as search for TP53 mutations.

The particular sets of primers designed in Example 1 lead to amplification and sequencing of productive clonal IGH-VDJ rearrangements from CLL cells gDNA in a very high proportion of cases (about 90% or 95% for gDNA samples sequenced using NGS or Sanger, respectively). Moreover, the careful and extensive protocol optimization lead to a signification reduction in amplification biases, an important feature for cases with more than one IGH-VDJ rearrangement.

An essential aspect of the proposed methodology is the complementary approach using cDNA template in case of failure or uncertainty with gDNA. As shown above, it allows to circumvent technical problems that may be encountered when using gDNA (most of them being due to somatic hypermutations on primer binding sites) and to obtain the sequence of the tumor productive IGH-VDJ rearrangement. Of note, the cDNA-based assays target all types of immunoglobulin constant region classes reported in CLL (IGH-Cmu, IGH-Cdelta, IGH-Cgamma). Thus, tumor sample of a given patient is preferably divided into two parts, one for gDNA extraction, and the other for storage for possible further RNA extraction and cDNA synthesis in case no productive clonal rearrangement can be sequenced starting from gDNA. Altogether this combined strategy allows the determination of IGHV mutational status assessment in virtually all cases of CLL.

Finally, the robustness of the proposed method was demonstrated by the high number of cases (several hundreds) on which it was evaluated, and very importantly the very good reproducibility when performed by other laboratories.

The assays presented here are well suited for being part of diagnostic kits, either for NGS or Sanger sequencing. Such kits could include reagents for both gDNA and cDNA (with a higher proportion of the former), or they could be purchased separately. Furthermore, the 47 plasmid mix constitutes a useful positive control to assess the quality of reagents and PCRs.

In conclusion, the assays presented here (especially those adapted to the increasingly used NGS technology) constitute a standardized methodology for determining IGHV mutational status in CLL. As this test is now mandatory for CLL prognostication and treatment choice, such standardization offers the possibility that it is performed accurately and in an optimal manner in all laboratories.

### Example 3: Addition of Further Amplification Primers for Sanger or NGS Determination of CLL Mutational Status on cDNA or Sanger Determination of CLL Mutational Status on gDNA

Further optimization of the primers sets defined in Examples 1 and 2 was performed in order to be able to further amplify rare CLL rearrangements.

### 1—Optimization of IGHV-L1 Primers

When comparing results obtained IGHV-L1 and IGHV-L2 primers on rare CLL cases expressing the IGHV3-64D gene, it became clear that the IGHV-L1 primer combination yielded poor results (see FIG. 13, upper part).

Careful examination of the IGHV3 primer (SEQ ID NO:26) sequence revealed that it had several mismatches with the corresponding region of IGHV3-64D gene.

We therefore tried several attempts to modify this primer either by removing the last 3′ nucleotide (SEQ ID NO:26_v2) and by incorporating degenerate nucleotides (SEQ ID NO:26_v3), but both approaches proved unsuccessful.

We then decided to add an additional specific primer (SEQ ID NO:133) (see Table 3 in general description above). Different PCR conditions were then evaluated on CLL cases having IGHV3-64D rearrangements, by varying the relative primer IGHV3-64D concentration until obtaining satisfactory results (see Table 4B in general description above), and then obtained satisfying detection of IGHV3-64D gene by NGS determination of CLL mutational status on cDNA (see FIG. 13, lower part).

While most of this optimization was performed by NGS on cDNA templates, a similarly satisfying detection of IGHV3-64D gene by Sanger determination of CLL mutational status on cDNA or gDNA is expected.

### 2—Optimization of IGHC Primers

Genomic DNA is the preferred source for determination of the IGHV mutational status as this template is robust, easy to prepare and also serves for oncogenic mutation screening (such as TP53) essential for CLL prognostication and guiding treatment decisions. In case of failure to detect a productive IGH rearrangement from gDNA, our alternative cDNA strategy can by-pass these problems, using primers in regions less (IGHL1) or not (IGHC) targeted by somatic hypermutations (the main cause of failure on gDNA).

As CLL are known to express mainly IgM or IgM+IgD and rarely (10%) IgG, we had designed our cDNA assay with 3′ constant region primers IGHC-mu, IGHC-delta and IGHC-gamma (see Examples 1 and 2).

However, in a few instances of gDNA failures, we were unable to obtain a productive rearrangement from cDNA templates using these constant region primers (see FIG. 14, left and middle sections). Therefore, we decided to incorporate an IGHC-alpha primer, although CLL are not known for expressing IgA. Optimization of the NGS-based cDNA assay was undertaken testing different IGHC-alpha primers, as well as different ratios and combinations of the IGHC primers.

An optimal primer (SEQ ID NO:134, see Table 3 in general description above) was found and its concentration with respect to other IGHC primers optimized (see This resulted in the ability to detect rare cases expressing only productive IGHV-IGHC-alpha rearrangements, thereby allowing their IGHV mutational status assessment (FIG. 14, right section).

### BIBLIOGRAPHIC REFERENCES

Agathangelidis A, Chatzidimitriou A, Gemenetzi K, et at. Higher-order connections between stereotyped subsets: implications for improved patient classification in CLL. Blood 2021: 137(10):1365-1376.
Armand M, Verrier P, Theves F, et at. Prevalence of IGLV3-21R110 among familial CLL: a retrospective study of 45 cases. Blood Adv. 2022 Jun. 28; 6(12):3632-3635.
Brochet X, Lefranc M P, Giudicelli V. IMGT/V-QUEST: the highly customized and integrated system for IG and TR standardized V-J and V-D-J sequence analysis. Nucleic Acids Res. 2008; 36(Web Server issue):W503-8.
Bystry V, Reigl T, Krejci A, et al. ARResT/Interrogate: an interactive immunoprofiler for IG/TR NGS data. Bioinformatics. 2017; 33(3):435-437.
Chai-Adisaksopha C, Brown J R. FCR achieves long-term durable remissions in patients with IGHV-mutated CLL. Blood. 2017; 130(21):2278-2282.
Damle R N, WasiI T, Fais F, et al. Ig V gene mutation status and CD38 expression as novel prognostic indicators in chronic lymphocytic leukemia. Blood. 1999; 94(6):1840-7.
Duez M, Giraud M, Herbert R, et al. Vidjil: A Web Platform for Analysis of High-Throughput Repertoire Sequencing. PLoS One. 2016; 11(11):e0166126.
Delpy L, Sirac C, Magnoux E, et al. RNA surveillance down-regulates expression of nonfunctional kappa alleles and detects premature termination within the last kappa exon. Proc Natl Acad Sci USA. 2004 May 11; 101(19):7375-80.
Eichhorst B, Robak T, Montserrat E, et al. Chronic Lymphocytic Leukaemia: ESMO Clinical Practice Guidelines for diagnosis, treatment and follow-up. Ann Oncol. 2021; 32(1):23-33.
Eichhorst B, Ghia P. EHA Endorsement of ESMO Clinical Practice Guidelines for Diagnosis, Treatment, and Follow-up of Chronic Lymphocytic Leukemia. Hemasphere. 2020; 5(1):e520
Filges, S., Yamada, E., Stihlberg, A. et al. Impact of Polymerase Fidelity on Background Error Rates in Next-Generation Sequencing with Unique Molecular Identifiers/Barcodes. Sci Rep 9, 3503 (2019).
Ghia P, Stamatopoulos K, Belessi C, et al. ERIC recommendations on IGHV gene mutational status analysis in chronic lymphocytic leukemia. Leukemia. 2007; 21 (1):1-3.
Gupta Sanjeev Kumar et al: “Evaluation of Somatic Hypermutation Status in Chronic Lymphocytic Leukemia (CLL) in the Era of Next Generation Sequencing”, FRONTIERS IN CELL AND DEVELOPMENTAL BIOLOGY, vol. 8, 19 May 2020 (2020 May 19). https://doi.org/10.3389/fcell.2020.00357.
Hallek M, Cheson B D, Catovsky D, et at. iwCLL guidelines for diagnosis, indications for treatment, response assessment, and supportive management of CLL. Blood. 2018; 131(25):2745-2760.
Hallek M, Shanafelt T D, Eichhorst B. Chronic lymphocytic leukaemia. Lancet. 2018; 391(10129): 1524-1537.
Hamblin T J, Davis Z, Gardiner A, et al. Unmutated Ig V(H) genes are associated with a more aggressive form of chronic lymphocytic leukemia. Blood. 1999; 94(6):1848-54.
Huet S, Bouvard A, Ferrant E, et al. Impact of using leader primers for IGHV mutational status assessment in chronic lymphocytic leukemia. Leukemia. 2020; 34(8):2257-2259.
Lefranc M P, Giudicelli V, Ginestoux C, et al. IMGT, the international ImMunoGeneTics information system. Nucleic Acids Res. 2009; 37(Database issue):D1006-12.
Langerak A W, Davi F, Ghia P, et al. Immunoglobulin sequence analysis and prognostication in CLL: guidelines from the ERIC review board for reliable interpretation of problematic cases. Leukemia. 2011; 25 (6), 979-984.
Lundberg K S, Shoemaker D D, Adams M W, et al. High-fidelity amplification using a thermostable DNA polymerase isolated from Pyrococcus furiosus. Gene. 1991; 108(1):1-6.
Maity P C, Bilal M, Koning M T, et al. IGLV3-21*01 is an inherited risk factor for CLL through the acquisition of a single-point mutation enabling autonomous BCR signaling. Proc Natl Acad Sci USA. 2020; 117(8):4320-4327.
McInerney P, Adams P, Hadi M Z. Error Rate Comparison during Polymerase Chain Reaction by DNA Polymerase. Hindawi Publishing Corporation. Mol Biol Int. 2014; 2014:287.
Minici C, Gounari M, €Ubelhart R, et al. Distinct homotypic B-cell receptor interactions shape the outcome of chronic lymphocytic leukaemia. Nat Commun. 2017; 8(1):15746.
Moreau E J, Matutes E, A'Hern R P, et al. Improvement of the chronic lymphocytic leukemia scoring system with the monoclonal antibodySN8 (CD79b). Am J Clin Pathol. 1997; 108(4): 378-82.
Nadeu F, Royo R, Clot G, et al. IGLV3-21R110 identifies an aggressive biological subtype of chronic lymphocytic leukemia with intermediate epigenetics. Blood. 2021; 137(21):2935-2946.
Rosenquist R, Ghia P, Hadzidimitriou A, et al. Immunoglobulin gene sequence analysis in chronic lymphocytic leukemia: updated ERIC recommendations. Leukemia. 2017; 31(7):1477-1481.
Schatz D G. V(D)J recombination. Immunol Rev. 2004; 200:5-11.
Stamatopoulos B. et al: “Targeted deep sequencing reveals clinically relevantsubclonal IgHV rearrangements in chronic lymphocytic leukemia”, LEUKEMIA, vol. 31, no. 4, 31 Oct. 2016 (2016 Oct. 31), pages 837-845.
STAMATOPOULOS K: “Immunoglobulin light chain repertoire in chronic lymphocytic leukemia”, BLOOD, vol. 106, no. 10, 15 Nov. 2005 (2005 Nov. 15), pages 3575-3583.
Stamatopoulos K, Agathangelidis A, Rosenquist R et at. Antigen receptor stereotypy in chronic lymphocytic leukemia. Leukemia. 2017; 31:282-291.
Sutton L A, Hadzidimitriou A, Baliakas P, et at. Immunoglobulin genes in chronic lymphocytic leukemia: key to understanding the disease and improving risk stratification. Haematologica. 2017; 102(6):968-971.
U.S. Pat. No. 6,127,155.
van Dongen J J, Langerak A W, Brüggemann M, et at. Design and standardization of PCR primers and protocols for detection of clonal immunoglobulin and T-cell receptor gene recombinations in suspect lymphoproliferations: report of the BIOMED-2 Concerted Action BMH4-CT98-3936. Leukemia. 2003; 17(12):2257-317.

1.-15. (canceled)

16. A kit for determining the mutational status of a patient suffering from B-cell chronic lymphocytic leukemia (CLL) from gDNA or cDNA extracted or obtained from a biological sample of said patient, said kit comprising:
a) forward primers comprising respectively the sequences SEQ ID NO:1 to SEQ ID NO:23 and a reverse primer comprising SEQ ID NO:30;
b) forward primers comprising respectively the sequences SEQ ID NO:24 to SEQ ID NO:29 and a reverse primer comprising SEQ ID NO:30;
c) forward primers comprising respectively the sequences SEQ ID NO:24 to SEQ ID NO:29 and reverse primers comprising respectively the sequences SEQ ID NO:31 to SEQ ID NO:33; and/or
d) nucleic acid molecules comprising respectively the sequences SEQ ID NO:76 to SEQ ID NO:122.

17. The kit according to claim 16, wherein said kit comprises forward primers comprising respectively the sequences SEQ ID NO:1 to SEQ ID NO:23 and a reverse primer comprising SEQ ID NO:30.

18. The kit according to claim 17, wherein said kit comprises forward primers consisting respectively, from 5′ to 3′, of a first adapter sequence fused to one of the sequences SEQ ID NO:1 to SEQ ID NO:23, and a reverse primer consisting, from 5′ to 3′, of a second adapter sequence fused to SEQ ID NO:30.

19. The kit according to claim 17, wherein said kit comprises:
a) forward primers comprising respectively the sequences SEQ ID NO:1 to SEQ ID NO:23;
b) a reverse primer comprising SEQ ID NO:30;
c) forward primers comprising respectively the sequences SEQ ID NO:24 to SEQ ID NO:29 or SEQ ID NO:24 to SEQ ID NO:29 and SEQ ID NO: 133; and
d) reverse primers comprising respectively the sequences SEQ ID NO:31 to SEQ ID NO:33 or SEQ ID NO:31 to SEQ ID NO:33 and SEQ ID NO: 134.

20. The kit according to claim 19, wherein said kit comprises:
a) forward primers consisting respectively, from 5′ to 3′, of a first adapter sequence fused to one of the sequences SEQ ID NO:1 to SEQ ID NO:23;
b) a reverse primer consisting, from 5′ to 3′ of a second adapter sequence fused to SEQ ID NO:30;
c) forward primers consisting respectively, from 5′ to 3′, of a third adapter sequence fused to one of the sequences SEQ ID NO:24 to SEQ ID NO:29 or SEQ ID NO:24 to SEQ ID NO:29 and SEQ ID NO:133; and
d) reverse primers consisting respectively, from 5′ to 3′, a fourth adapter sequence fused to one of the sequences SEQ ID NO:31 to SEQ ID NO:33 or SEQ ID NO:31 to SEQ ID NO:33 and SEQ ID NO:133;
said kit further comprising nucleic acid molecules comprising respectively the sequences SEQ ID NO:76 to SEQ ID NO:122.

21. The kit according to claim 16, wherein said kit comprises forward primers comprising respectively the sequences SEQ ID NO:24 to SEQ ID NO:29 or SEQ ID NO:24 to SEQ ID NO:29 and SEQ ID NO:133 and a reverse primer comprising SEQ ID NO:30.

22. The kit according to claim 21, wherein said kit comprises forward primers consisting respectively of the sequences SEQ ID NO:24 to SEQ ID NO:29 or SEQ ID NO:24 to SEQ ID NO:29 and SEQ ID NO:133 and a reverse primer consisting of SEQ ID NO:30.

23. The kit according to claim 21, wherein said kit comprises:
a) forward primers comprising respectively the sequences SEQ ID NO:24 to SEQ ID NO:29 or SEQ ID NO: 24 to SEQ ID NO: 29 and SEQ ID NO:133
b) a reverse primer comprising SEQ ID NO:30 or a reverse primer consisting of SEQ ID NO:30; and
c) reverse primers comprising respectively the sequences SEQ ID NO:31 to SEQ ID NO:33 or SEQ ID NO:31 to SEQ ID NO:33 and SEQ ID NO:134.

24. The kit according to claim 23, wherein said kit comprises:
a) forward primers consisting respectively of the sequences SEQ ID NO:24 to SEQ ID NO:29 or of the sequences SEQ ID NO:24 to SEQ ID NO: 29 and SEQ ID NO:133;
b) a reverse primer comprising SEQ ID NO:30 or a reverse primer consisting of SEQ ID NO:30; and
c) reverse primers consisting respectively of the sequences SEQ ID NO:31 to SEQ ID NO:33 or of the sequences SEQ ID NO:31 to SEQ ID NO:33 and SEQ ID NO:134;
said kit further comprising nucleic acid molecules comprising respectively le sequences SEQ ID NO:76 to SEQ ID NO:122.

25. The kit according to claim 16, wherein:
i) forward primers comprising respectively the sequences SEQ ID NO:1 to SEQ ID NO:23 are mixed in a single solution;
ii) forward primers comprising respectively the sequences SEQ ID NO:24 to SEQ ID NO:29 or SEQ ID NO:24 to SEQ ID NO:29 and SEQ ID NO:133 are mixed in a single solution;
iii) reverse primers comprising respectively the sequences SEQ ID NO:31 to SEQ ID NO:33 are mixed in a single solution;
iv) forward primers comprising respectively the sequences SEQ ID NO:24 to SEQ ID NO:29 or SEQ ID NO:24 to SEQ ID NO:29 and SEQ ID NO:133 and reverse primer comprising SEQ ID NO:30 are mixed in a single solution;
v) forward primers comprising respectively the sequences SEQ ID NO:24 to SEQ ID NO:29 or SEQ ID NO:24 to SEQ ID NO:29 and SEQ ID NO:133 and reverse primers comprising respectively the sequences SEQ ID NO:31 to SEQ ID NO:33 are mixed in a single solution;
vi) reverse primers comprising respectively the sequences SEQ ID NO:31 and SEQ ID NO:33 are mixed in a first single solution and the reverse primer comprising the sequence SEQ ID NO:32 is in another second solution;
vii) reverse primers comprising respectively the sequences SEQ ID NO:31 and SEQ ID NO:33 are mixed in a first single solution and reverse comprising respectively the sequences SEQ ID NO:32 and SEQ ID NO:134 are mixed in a second single solution;
viii) forward primers comprising respectively the sequences SEQ ID NO:24 to SEQ ID NO:29 or SEQ ID NO:24 to SEQ ID NO:29 and SEQ ID NO:133 and reverse primers comprising respectively the sequences SEQ ID NO:31 and SEQ ID NO:33 are mixed in a first single solution and forward primers comprising respectively the sequences SEQ ID NO:24 to SEQ ID NO:29 or SEQ ID NO:24 to SEQ ID NO:29 and SEQ ID NO:133 and the reverse primer comprising the sequence SEQ ID NO:32 are mixed in a second single solution;
ix) forward primers comprising respectively the sequences SEQ ID NO:24 to SEQ ID NO:29 or SEQ ID NO:24 to SEQ ID NO:29 and SEQ ID NO:133 and reverse primers comprising respectively the sequences SEQ ID NO:31 and SEQ ID NO:33 are mixed in a first single solution, and forward primers comprising respectively the sequences SEQ ID NO:24 to SEQ ID NO:29 or SEQ ID NO:24 to SEQ ID NO:29 and SEQ ID NO:133 and reverse primers comprising respectively the sequences SEQ ID NO:32 and SEQ ID NO:134 are mixed in a second single solution; and/or
x) Nucleic acid molecules comprising respectively le sequences SEQ ID NO:76 to SEQ ID NO:122 are mixed in a single solution.

26. The kit according to claim 16, which further comprises:
a) instructions of use for determining the mutational status of a patient suffering from CLL,
b) a high fidelity DNA polymerase and its buffer, or
c) a dNTP mix,
d) a MgSO4 solution,
e) nuclease-free water, or
f) any combination of a) to e).

27. A method for determining the mutational status of a patient suffering from B-cell chronic lymphocytic leukemia (CLL) from a biological sample of said CLL patient, comprising the steps of:
a) obtaining genomic DNA (gDNA) and/or complementary DNA (cDNA) from the biological sample;
b) amplifying rearranged immunoglobulin heavy chain genes from gDNA and/or cDNA by multiplex polymerase chain reaction (PCR) using primers from the kit according to claim 16;
c) sequencing amplified rearranged heavy chain immunoglobulin genes using either Sanger or NGS sequencing depending on the composition of the kit and identifying a clonal productive rearranged heavy chain immunoglobulin gene;
d) aligning the identified clonal productive rearranged heavy chain immunoglobulin gene to germline immunoglobulin IGHV, IGHD and IGHJ genes, determining the percentage of identity between the IGHV gene of the identified clonal productive rearranged heavy chain immunoglobulin gene and its closest germline immunoglobulin IGHV gene; and
e) determining the mutational status of the CLL patient, wherein the mutational status is:
unmutated if the percentage of identity between the IGHV gene of the identified clonal productive rearranged heavy chain immunoglobulin gene and its closest germline immunoglobulin IGHV gene is equal or higher than 98%, and
mutated if the percentage of identity between the IGHV gene of the identified clonal productive rearranged heavy chain immunoglobulin gene and its closest germline immunoglobulin IGHV gene is below 98%.

28. The method of claim 27, wherein said method comprises:
a) extracting genomic DNA (gDNA) from a first part of the biological sample and freezing the second part of the biological sample,
b) amplifying rearranged immunoglobulin heavy chain genes from gDNA by multiplex polymerase chain reaction (PCR) with the following primers:
i) forward primers:
Forward primers comprising respectively the target sequences SEQ ID NO:1 to SEQ ID NO:23 if step c) is to be performed by NGS sequencing; or
Forward primers comprising respectively the target sequences SEQ ID NO:24 to SEQ ID NO:29 or SEQ ID NO:24 to SEQ ID NO:29 and SEQ ID NO:133, if step c) is to be performed by Sanger sequencing; and
ii) a reverse primer comprising the target sequence SEQ ID NO:30;
c) sequencing amplified rearranged heavy chain immunoglobulin genes using either Sanger or NGS sequencing and identifying a clonal productive rearranged heavy chain immunoglobulin gene,
d) aligning the identified clonal productive rearranged heavy chain immunoglobulin gene to germline immunoglobulin IGHV, IGHD and IGHJ genes, determining the percentage of identity between the IGHV gene of the identified clonal productive rearranged heavy chain immunoglobulin gene and its closest germline immunoglobulin IGHV gene, and
e) determining the mutational status of the CLL patient, wherein the mutational status is:
unmutated if the percentage of identity between the IGHV gene of the identified clonal productive rearranged heavy chain immunoglobulin gene and its closest germline immunoglobulin IGHV gene is equal or higher than 98%, and
mutated if the percentage of identity between the IGHV gene of the identified clonal productive rearranged heavy chain immunoglobulin gene and its closest germline immunoglobulin IGHV gene is below 98%.

29. The method according to claim 28, wherein said biological sample is a blood sample, a bone marrow sample, a lymph node sample, or any tissue sample infiltrated by CLL cells and wherein said second part of the biological sample is frozen as a dry cells' pellet or as a cell lysate after extraction with a lysis solution comprising a chaotropic agent.

30. The method according to claim 28, wherein a high fidelity DNA polymerase is used in step b).

31. The method according to claim 28, wherein:
a) when step c) is performed by NGS sequencing, the forward primers consist respectively, from 5′ to 3′, of a first adapter sequence fused to one of the sequences SEQ ID NO:1 to SEQ ID NO:23, and the reverse primer consists, from 5′ to 3′, of a second adapter sequence fused to SEQ ID NO:30; or
b) when step c) is performed by Sanger sequencing, the forward primers consist respectively of the sequences SEQ ID NO:24 to SEQ ID NO:29 or of the sequences SEQ ID NO:24 to SEQ ID NO:29 and SEQ ID NO:133 and the reverse primer consists of SEQ ID NO:30.

32. The method according to claim 28, wherein when a clonal productive IGVH rearrangement is not sequenced in step c) the method further comprises between step c) and step d) the additional steps of:
c1) extracting RNA from the second part of the biological sample and converting it to complementary DNA (cDNA) or providing cDNA previously obtained from the second part of the biological sample,
c2) amplifying rearranged immunoglobulin heavy chain genes from cDNA by multiplex polymerase chain reaction (PCR) with the following primers:
i) Forward primers comprising respectively the target sequences SEQ ID NO:24 to SEQ ID NO:29 or SEQ ID NO:24 to SEQ ID NO:29 and SEQ ID NO:133, and
ii) Reverse primers comprising respectively the target sequences SEQ ID NO:31 to SEQ ID NO:33 or SEQ ID NO:31 to SEQ ID NO:33 and SEQ ID NO:134, and
c3) sequencing amplified rearranged heavy chain immunoglobulin genes using either Sanger or NGS sequencing and identifying a clonal productive rearranged heavy chain immunoglobulin gene,
wherein step d) is performed on the clonal productive rearranged heavy chain immunoglobulin gene identified in step c3).

33. The method according to claim 32, wherein:
b) when step c) is performed by NGS sequencing, the forward primers consist respectively, from 5′ to 3′, of a first adapter sequence fused to one of the sequences SEQ ID NO:24 to SEQ ID NO:29 or SEQ ID NO:24 to SEQ ID NO:29 and SEQ ID NO:133, and the reverse primer consists, from 5′ to 3′, of a second adapter sequence fused to one of the sequences SEQ ID NO:31 to SEQ ID NO:33 or SEQ ID NO:31 to SEQ ID NO:33 and SEQ ID NO:134; or
a) when step c) is performed by Sanger sequencing, the forward primers consist respectively of the sequences SEQ ID NO:24 to SEQ ID NO:29 or SEQ ID NO:24 to SEQ ID NO:29 and SEQ ID NO:133 and the reverse primers consist respectively of the sequences SEQ ID NO:31 to SEQ ID NO:33 or SEQ ID NO:31 to SEQ ID NO:33 and SEQ ID NO:134.

34. The kit according to claim 21 when step c) is performed by NGS sequencing, wherein:
i) suitable adapter sequences for forward primers have the following structure:
5′-Forward flow cell binding adapter-Index i5-Forward sequencing primer site-3′,
wherein:
the Forward flow cell binding adapter is of sequence
(SEQ ID NO: 34)
AATGATACGGCGACCACCGAGATCTACAC,
the Forward sequencing primer site is of sequence
(SEQ ID NO: 35)
ACACTCTTTCCCTACACGACGCTCTTCCGATCT,
and
the Index i5 is selected from one of the sequences SEQ ID NO: 36 to 53, and
ii) suitable adapter sequences for forward primers have the following structure:
5′-Reverse flow cell binding adapter-Index i7—Reverse sequencing primer site-3′,
wherein:
the Reverse flow cell binding adapter is of sequence
(SEQ ID NO: 54)
CAAGCAGAAGACGGCATACGAGAT,
the Reverse sequencing primer site is of sequence
(SEQ ID NO: 55)
GTGACTGGAGTTCAGACGTGTGCTCTTCCGATCT,
and
the Index i7 is selected from one of the sequences SEQ ID NO:56 to 75.

35. The method according to claim 31 when step c) is performed by NGS sequencing, wherein:
iii) suitable adapter sequences for forward primers have the following structure:
5′-Forward flow cell binding adapter-Index i5-Forward sequencing primer site-3′,
wherein:
the Forward flow cell binding adapter is of sequence
(SEQ ID NO: 34)
AATGATACGGCGACCACCGAGATCTACAC,
the Forward sequencing primer site is of sequence ACACTCTTTCCCTACACGACGCTCTTCCGATCT (SEQ ID NO:35), and
the Index i5 is selected from one of the sequences SEQ ID NO: 36 to 53, and
iv) suitable adapter sequences for forward primers have the following structure:
5′-Reverse flow cell binding adapter-Index i7—Reverse sequencing primer site-3′,
wherein:
the Reverse flow cell binding adapter is of sequence
(SEQ ID NO: 54)
CAAGCAGAAGACGGCATACGAGAT,
the Reverse sequencing primer site is of sequence GTGACTGGAGTTCAGACGTGTGCTCTTCCGATCT (SEQ ID NO:55), and
the Index i7 is selected from one of the sequences SEQ ID NO:56 to 75.


SEQUENCE LISTING (134 sequences)

SEQ ID NO: 1 - DNA, 20 bp, synthetic construct
  misc_feature 1..20: note = Forward primer IGHV-L2-1.1
  source 1..20: mol_type = other DNA
gtgttctctccacaggagcc

SEQ ID NO: 2 - DNA, 22 bp, synthetic construct
  misc_feature 1..22: note = Forward primer IGHV-L2-1.2
  source 1..22: mol_type = other DNA
gtgtcttctctacaggtgccca

SEQ ID NO: 3 - DNA, 20 bp, synthetic construct
  misc_feature 1..20: note = Forward primer IGHV-L2-1.3
  source 1..20: mol_type = other DNA
gtgttctctccacaggtgcc

SEQ ID NO: 4 - DNA, 20 bp, synthetic construct
  misc_feature 1..20: note = Forward primer IGHV-L2-1.4
  source 1..20: mol_type = other DNA
gtgtcctctccacaggtgcc

SEQ ID NO: 5 - DNA, 20 bp, synthetic construct
  misc_feature 1..20: note = Forward primer IGHV-L2-1.5
  source 1..20: mol_type = other DNA
ctgtcctctccacaggcacc

SEQ ID NO: 6 - DNA, 19 bp, synthetic construct
  misc_feature 1..19: note = Forward primer IGHV-L2-1.6
  source 1..19: mol_type = other DNA
gtgtcccctccacagatgc

SEQ ID NO: 7 - DNA, 18 bp, synthetic construct
  misc_feature 1..18: note = Forward primer IGHV-L2-1.7
  source 1..18: mol_type = other DNA
gtgtcctctccgcaggtg

SEQ ID NO: 8 - DNA, 26 bp, synthetic construct
  misc_feature 1..26: note = Forward primer IGHV-L2-1.8_v9
  source 1..26: mol_type = other DNA
gtgtcctctccacaggtgtccagtcc

SEQ ID NO: 9 - DNA, 20 bp, synthetic construct
  misc_feature 1..20: note = Forward primer IGHV-L2-1.9
  source 1..20: mol_type = other DNA
ttctcttctccacaggcacc

SEQ ID NO: 10 - DNA, 23 bp, synthetic construct
  misc_feature 1..23: note = Forward primer IGHL2-2.1
  source 1..23: mol_type = other DNA
cttatgtcttctccacaggggtc

SEQ ID NO: 11 - DNA, 22 bp, synthetic construct
  misc_feature 1..22: note = Forward primer IGHL2-2.2
  source 1..22: mol_type = other DNA
cttatgctttctccacaggggt

SEQ ID NO: 12 - DNA, 21 bp, synthetic construct
  misc_feature 1..21: note = Forward primer IGHL2-3.1
  source 1..21: mol_type = other DNA
tgtgtttgcaggtgtccagtg

SEQ ID NO: 13 - DNA, 21 bp, synthetic construct
  misc_feature 1..21: note = Forward primer IGHL2-3.2
  source 1..21: mol_type = other DNA
tctgtttgcaggtgtccagtg

SEQ ID NO: 14 - DNA, 21 bp, synthetic construct
  misc_feature 1..21: note = Forward primer IGHL2-3.3
  source 1..21: mol_type = other DNA
tttgtttgcaggtgtccagtg

SEQ ID NO: 15 - DNA, 21 bp, synthetic construct
  misc_feature 1..21: note = Forward primer IGHL2-3.4
  source 1..21: mol_type = other DNA
tgtgtttgcaggtgtccaatg

SEQ ID NO: 16 - DNA, 20 bp, synthetic construct
  misc_feature 1..20: note = Forward primer IGHL2-3.5
  source 1..20: mol_type = other DNA
cgtgtttgcaggtgtccagt

SEQ ID NO: 17 - DNA, 21 bp, synthetic construct
  misc_feature 1..21: note = Forward primer IGHL2-3.6
  source 1..21: mol_type = other DNA
tttgtttgcagatgtccagtg

SEQ ID NO: 18 - DNA, 22 bp, synthetic construct
  misc_feature 1..22: note = Forward primer IGHL2-4.1
  source 1..22: mol_type = other DNA
gtctctctgttcacaggggtcc

SEQ ID NO: 19 - DNA, 22 bp, synthetic construct
  misc_feature 1..22: note = Forward primer IGHL2-4.2
  source 1..22: mol_type = other DNA
gtttctctgttcacaggggtcc

SEQ ID NO: 20 - DNA, 22 bp, synthetic construct
  misc_feature 1..22: note = Forward primer IGHL2-4.3
  source 1..22: mol_type = other DNA
gtttttctgttcacaggggtcc

SEQ ID NO: 21 - DNA, 20 bp, synthetic construct
  misc_feature 1..20: note = Forward primer IGHL2-5.1
  source 1..20: mol_type = other DNA
tctcccccacaggagtctgt

SEQ ID NO: 22 - DNA, 22 bp, synthetic construct
  misc_feature 1..22: note = Forward primer IGHL2-5.2
  source 1..22: mol_type = other DNA
tcttccatacaggagtctgtgc

SEQ ID NO: 23 - DNA, 21 bp, synthetic construct
  misc_feature 1..21: note = Forward primer IGHL2-6.1
  source 1..21: mol_type = other DNA
tgtctccaggtgtcctgtcac

SEQ ID NO: 24 - DNA, 23 bp, synthetic construct
  misc_feature 1..23: note = Forward primer IGHL1-1
  source 1..23: mol_type = other DNA
ctcaccatggactgsayytggag

SEQ ID NO: 25 - DNA, 25 bp, synthetic construct
  misc_feature 1..25: note = Forward primer IGHL1-2
  source 1..25: mol_type = other DNA
atggacayactttgytmcacrctcc

SEQ ID NO: 26 - DNA, 25 bp, synthetic construct
  misc_feature 1..25: note = Forward primer IGHL1-3
  source 1..25: mol_type = other DNA
atggarttkgggctkwgctgggttt

SEQ ID NO: 27 - DNA, 25 bp, synthetic construct
  misc_feature 1..25: note = Forward primer IGHL1-4
  source 1..25: mol_type = other DNA
ctgtggttcttyctbctsctggtgg

SEQ ID NO: 28 - DNA, 23 bp, synthetic construct
  misc_feature 1..23: note = Forward primer IGHL1-5
  source 1..23: mol_type = other DNA
cctcctcctrgctrttctccaag

SEQ ID NO: 29 - DNA, 25 bp, synthetic construct
  misc_feature 1..25: note = Forward primer IGHL1-6
  source 1..25: mol_type = other DNA
ctgtctccttcctcatcttcctgcc

SEQ ID NO: 30 - DNA, 22 bp, synthetic construct
  misc_feature 1..22: note = Reverse primer IGHJ
  source 1..22: mol_type = other DNA
cttacctgaggagacggtgacc

SEQ ID NO: 31 - DNA, 18 bp, synthetic construct
  misc_feature 1..18: note = Reverse primer IGHC-mu
  source 1..18: mol_type = other DNA
ggttggggcggatgcact

SEQ ID NO: 32 - DNA, 21 bp, synthetic construct
  misc_feature 1..21: note = Reverse primer IGHC-delta
  source 1..21: mol_type = other DNA
gggaacacatccggagccttg

SEQ ID NO: 33 - DNA, 18 bp, synthetic construct
  misc_feature 1..18: note = Reverse primer IGHC-gamma
  source 1..18: mol_type = other DNA
cgatgggcccttggtgga

SEQ ID NO: 34 - DNA, 29 bp, synthetic construct
  misc_feature 1..29: note = Forward flow cell binding adapter
  source 1..29: mol_type = other DNA
aatgatacggcgaccaccgagatctacac

SEQ ID NO: 35 - DNA, 33 bp, synthetic construct
  misc_feature 1..33: note = Forward sequencing primer site
  source 1..33: mol_type = other DNA
acactctttccctacacgacgctcttccgatct

SEQ ID NO: 36-53 - skipped (18 entries)

SEQ ID NO: 54 - DNA, 24 bp, synthetic construct
  misc_feature 1..24: note = Reverse flow cell binding adapter
  source 1..24: mol_type = other DNA
caagcagaagacggcatacgagat

SEQ ID NO: 55 - DNA, 34 bp, synthetic construct
  misc_feature 1..34: note = Reverse sequencing primer site
  source 1..34: mol_type = other DNA
gtgactggagttcagacgtgtgctcttccgatct

SEQ ID NO: 56-75 - skipped (20 entries)

SEQ ID NO: 76 - DNA, 361 bp, synthetic construct
  misc_feature 1..361: note = Internal control P01
  source 1..361: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgaggtggcagtggctggtactaggatactttgactactggggccagggaaccctggtcaccgtctcctcag

SEQ ID NO: 77 - DNA, 372 bp, synthetic construct
  misc_feature 1..372: note = Internal control P02
  source 1..372: mol_type = other DNA
caggtccagcttgtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgagaatgtatagtgggagatcttactactactactacggtatggacgtctggggccaagggaccacggtcaccgtctcctca

SEQ ID NO: 78 - DNA, 397 bp, synthetic construct
  misc_feature 1..397: note = Internal control P03
  source 1..397: mol_type = other DNA
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagaggcctccggattgtagtagtaccagctgcatacaacggggattactactactactacggtatggacgtctggggccaagggaccacggtcaccgtctcctcag

SEQ ID NO: 79 - DNA, 402 bp, synthetic construct
  misc_feature 1..402: note = Internal control P04
  source 1..402: mol_type = other DNA
caggttcagctggtgcagtctggagctgaggtgaagaagcctggggcctcagtgaaggtctcctgcaaggcttctggttacacctttaccagctacggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagtgaatgggtgggatgtagtagtaccagccactatacccgccgagagctactactactactacggtatggacgtctggggccaagggaccacggtcaccgtctcctca

SEQ ID NO: 80 - DNA, 361 bp, synthetic construct
  misc_feature 1..361: note = Internal control P05
  source 1..361: mol_type = other DNA
caggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacagcaggctattttgactggttccattttgactactggggccagggaaccctggtcaccgtctcctcag

SEQ ID NO: 81 - DNA, 390 bp, synthetic construct
  misc_feature 1..390: note = Internal control P06
  source 1..390: mol_type = other DNA
cagatgcagctggtgcagtctggggctgaggtgaagaagactgggtcctcagtgaaggtttcctgcaaggcttccggatacaccttcacctaccgctacctgcactgggtgcgacaggcccccggacaagcgcttgagtggatgggatggatcacacctttcaatggtaacaccaactacgcacagaaattccaggacagagtcaccattaccagggacaggtctatgagcacagcctacatggagctgagcagcctgagatctgaggacacagccatgtattactgtgcaagcgatataggggggagggctattactatggttcggggagttaactactactacggtatggacgtctggggccaagggaccacggtcaccgtctcctca

SEQ ID NO: 82 - DNA, 376 bp, synthetic construct
  misc_feature 1..376: note = Internal control P07
  source 1..376: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagatattgtagtggtggtagctgcgctagaggcggggtggttcgacccctggggccagggaaccctggtcaccgtctcctcag

SEQ ID NO: 83 - DNA, 378 bp, synthetic construct
  misc_feature 1..378: note = Internal control P08
  source 1..378: mol_type = other DNA
caaatgcagctggtgcagtctgggcctgaggtgaagaagcctgggacctcagtgaaggtctcctgcaaggcttccggattcacttttactgactctgctgtgcagtgggtgcgtcaggctcgtggacaacgccttgagtggataggatggatcgtcgttggcagtgctaacacaaactacgcacagaagttccaggagagagtcaccattaccagggacatgtccacaagcacagcctacatggagctgagcagcctgagatccgaggacacggccgtctattactgtgcggcagggggtgtgggtggattctggacttcttactactactactacatggacgtctggggcaaagggaccacggtcaccgtctcctca

SEQ ID NO: 84 - DNA, 390 bp, synthetic construct
  misc_feature 1..390: note = Internal control P09
  source 1..390: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgacgacgtattactatgatagtagtggttattactgggttgatgactactactacggtatggacgtctggggccaagggaccacggtcaccgtctcctca

SEQ ID NO: 85 - DNA, 338 bp, synthetic construct
  misc_feature 1..338: note = Internal control P10
  source 1..338: mol_type = other DNA
gaggtccggctggtacaatctggggctgaggtgaagaagcctggggctacagtgaaaatctcctgcaaggtttctggatacaccttcaccgactactacatccactgggtgcgacaggcccctggaaaagggcttgagtggatgggacttgttgatcctgaagatggtgaaacaacatacgcagagaagttccagggcagagtcaccataaccgcggacacgtctacagacacagcctatatggaggtgcgcagcctgagatctgaagacacggccgtgtattattgtgcaaccattttgagtgattatttggttgacttctggggccagggaaccct

SEQ ID NO: 86 - DNA, 376 bp, synthetic construct
  misc_feature 1..376: note = Internal control P11
  source 1..376: mol_type = other DNA
cagatcaccttgaaggagtctggtcctacgctggtgaaacccacacagaccctcacgctgacctgcaccgtctctgggttctcagtcagcgctagtggagtgagtgtgggctggatccgtcagcccccaggaaaggccctggaatggcttgcactcatttattgggatgatgataagcgctacagcccgtctctgaaaagcaggctcaccatcaccaaggacatctccaaaaaccacgtggtccttacaatgaccaacatggaccctatggacacagccacatattattgtgcacgcagggcggggtatgactggaattacgagcgtgcctggttcgacccctggggccagggaatcccggtcaccgtctcctcag

SEQ ID NO: 87 - DNA, 364 bp, synthetic construct
  misc_feature 1..364: note = Internal control P12
  source 1..364: mol_type = other DNA
caggtcaccttgagggagtctggtcctgtgctggtgaaacccacagagaccctcacgctgacctgcaccgtctctgggttctcactcagcaatgttggaatgggtgtgagctggatccgtcagcccccagggaaggccctggaatggcttgcacacattttttcgaatgacgaaaaatcctgcagcacatctctgaagagcaggctcaccatctccaaggacacctccaaaagccacgtggtccttatcatgaccaacatggaccctgtggacacagccacatattactgtgcacggagtagtagtatcagcgatgatgcttttgatgtctggggccaagggacaatggtcaccgtctcctcag

SEQ ID NO: 88 - DNA, 391 bp, synthetic construct
  misc_feature 1..391: note = Internal control P13
  source 1..391: mol_type = other DNA
caggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattactgtgcacggatacggtatagcagcagctggtccccagggccttggtactactactacggtatggacgtctggggccaagggaccacggtcaccgtctcctcag

SEQ ID NO: 89 - DNA, 397 bp, synthetic construct
  misc_feature 1..397: note = Internal control P14
  source 1..397: mol_type = other DNA
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagaacccgattggggaatcagatcattactatggttcggggagttatttgggcccgatgcttttgatatctggggccaagggacaatggtcaccgtctcctcag

SEQ ID NO: 90 - DNA, 402 bp, synthetic construct
  misc_feature 1..402: note = Internal control P15
  source 1..402: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagcggccccggaagaccactattgtagtggtggtagctgctaccccaattactactactactacggtatggacgtctggggccaagggaccacggtcaccgtctcctca

SEQ ID NO: 91 - DNA, 387 bp, synthetic construct
  misc_feature 1..387: note = Internal control P16
  source 1..387: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagagcccactttcccctctccgtttgggagtggtcccatagctactactacggtatggacgtctggggccaagggaccacggtcaccgtctcctca

SEQ ID NO: 92 - DNA, 348 bp, synthetic construct
  misc_feature 1..348: note = Internal control P17
  source 1..348: mol_type = other DNA
gaggtgcagctggtggagtctgggggaggcttggtacagcctggggggtccctgagactctcctgtgcagcctgtggattcaccttcagtagctacgacatgcactgggtccgccaagctacaggaaaaggtctggagtgggtctcagctattggtactgctggtgacacatactatccaggctccgtgaagggccaattcaccatctccagagaaaatgccaagaactccttgtatcttcaaatgaacagcctgagagccggggacacggctgtgtattactgtgcaagagatggacatgatatggggaactactggggccggggaaccctggtcaccctctcctca

SEQ ID NO: 93 - DNA, 402 bp, synthetic construct
  misc_feature 1..402: note = Internal control P18
  source 1..402: mol_type = other DNA
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagtacgtttaaacgagaacgatttttggagtggtaattttgaggtccactactactacggtatggacgtctggggccaagggaccacggtcaccgtctcctca

SEQ ID NO: 94 - DNA, 376 bp, synthetic construct
  misc_feature 1..376: note = Internal control P19
  source 1..376: mol_type = other DNA
gaggtgcagctggtggagtctgggggaggtgtggtacggcctggggggtccctgagactctcctgtgcagcctctggattcacctttgatgattatgccatgagctgggtccgccaagctccaggggcggggctggagtgggtctctggtattcattggaacggtggtggcacaggttatgcagactctgtgcagggccgattcaccatctccagagacaacgccaagaactccctgtatttgcaaatgaacagtctgagagtcgaagacacggccttctattactgtgcgagagttggcggtgggggtatagcagttgctggtacaaactggctcgacccctggggccagggaatcctggtcaccgtctcctcag

SEQ ID NO: 95 - DNA, 387 bp, synthetic construct
  misc_feature 1..387: note = Internal control P20
  source 1..387: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgaggaaatactatgatagtagtggttattactactggcaagactactactacggtatggacgtctggggccaagggaccacggtcaccgtctcctca

SEQ ID NO: 96 - DNA, 402 bp, synthetic construct
  misc_feature 1..402: note = Internal control P21
  source 1..402: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagattcggggatgcagtattacgatttttggagtggttatcaccccgcctactactactactacggtatggacgtctggggccaagggaccacggtcaccgtctcctca

SEQ ID NO: 97 - DNA, 379 bp, synthetic construct
  misc_feature 1..379: note = Internal control P22
  source 1..379: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagggattcgaaggcttccttattactatgatagtagtggtccgttcgacccctggggccagggaaccctggtcaccgtctcctcag

SEQ ID NO: 98 - DNA, 388 bp, synthetic construct
  misc_feature 1..388: note = Internal control P23
  source 1..388: mol_type = other DNA
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagattttttgcagtggctggtacctggtgcatattactactactactacggtatggacgtctggggccaagggaccacggtcaccgtctcctcag

SEQ ID NO: 99 - DNA, 385 bp, synthetic construct
  misc_feature 1..385: note = Internal control P24
  source 1..385: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagatccgggtttgtacggttgcctggctagtagtaccagctgctatccctttgactactggggccagggaaccctggtcaccgtctcctcag

SEQ ID NO: 100 - DNA, 352 bp, synthetic construct
  misc_feature 1..352: note = Internal control P25
  source 1..352: mol_type = other DNA
gaggtgcagctggtggagtctcggggagtcttggtacagcctggggggtccctgagactctcctgtgcagcctctggattcaccgtcagtagcaatgagatgagctgggtccgccaggctccagggaagggtctggagtgggtctcatccattagtggtggtagcacatactacgcagactccaggaagggcagattcaccatctccagagacaattccaagaacacgctgcatcttcaaatgaacagcctgagagctgaggacacggctgtgtattactgtacggggtggttcgggaggatactccaatatggctactggggccagggaaccctggtcaccgtctcctcag

SEQ ID NO: 101 - DNA, 378 bp, synthetic construct
  misc_feature 1..378: note = Internal control P26
  source 1..378: mol_type = other DNA
gaagtgcagatggtggaatctggaggaggcgtggagcagcctggggggtccctgagactctcctgcgcaacgtctggattcaactttgatgattattccatgcactgggtccgtcaagctccagggaagggtctggagtggatctctcttattactggggacggtgataccacatcctatgcagactctgtgaaggacagattcatcatctccagagacaaccgcaaaaactccctgtatctgcaaatgtacagtctgacaattgaggacaccgccttctattattgtacacgaggcggaattaaaaaggccgatagtgggagctactatcgatggctcggcccctggggccagggaaccctggtcaccgtctcctca

SEQ ID NO: 102 - DNA, 384 bp, synthetic construct
  misc_feature 1..384: note = Internal control P27
  source 1..384: mol_type = other DNA
gaggtgcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtagtggtagtaccatatactacgcagactctgtgaagggccgattcaccatctccagagacaacgccaagaactcactgtatctgcaaatgaacagcctgagagccgaggacacggctgtttattactgtgcgagaggctacgatttttggagtggttacctctcatactactactactacggtatggacgtctggggccaagggaccacggtcaccgtctcctca

SEQ ID NO: 103 - DNA, 393 bp, synthetic construct
  misc_feature 1..393: note = Internal control P28
  source 1..393: mol_type = other DNA
gaggtgcagctggtggagtctgggggaggcttggtaaagccagggcggtccctgagactctcctgtacagcttctggattcacctttggtgattatgctatgagctggttccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactatatcggatatggttcattacgatttttggagtggttactattcggggggctggttcgacccctggggccagggaaccctggtcaccgtctcctca

SEQ ID NO: 104 - DNA, 355 bp, synthetic construct
  misc_feature 1..355: note = Internal control P29
  source 1..355: mol_type = other DNA
gaggtgcagctggtggagtctggaggaggcttgatccaccctggggggtccctgagactctcttgtgcagcctctgggttcaccgtcagtcacaacgacatgagctgggtccgccaggctccagggaaggggctggagtgggtctcagttatttatagcggtggtaacacatactacgcagactccgtgaagggccgattcaccatctccagagacaattccaagaacacgctggatcttcaaatgaacagcctgagagccgaggacacggccgtgtattactgtgcgagaacgcctgggggtagtggttatattgactactggggccagggaaccctggtcaccgtctcctcag

SEQ ID NO: 105 - DNA, 379 bp, synthetic construct
  misc_feature 1..379: note = Internal control P30
  source 1..379: mol_type = other DNA
gaggtgcagctggtggagtctgggggaggcttggtccagcctggggggtccctgagactctcttgttcagcctctggattccccttcagtacctatgctatccactgggtccgccaggctccagggaagggactggagtatgtttcagctattaatagtgatgggggtagcacctactacgcagactccgtgaaggacagattcaccatctccagagacaattccaagaacactctgtatcttcaaatgaacagtctgagaactgaggacacggctgtctattactgtgttaaaggtgacatcggggaattatcaatattatactactactacggtttgcacgtctggggccaggggaccgcggtcaccgtctcctcag

SEQ ID NO: 106 - DNA, 355 bp, synthetic construct
  misc_feature 1..355: note = Internal control P31
  source 1..355: mol_type = other DNA
gaggtgcagctggtggagtctgggggaggcttggtccagcctggggggtccctgagactctcctgtgcagcctctggattcaccgtcagcagcaactacatgaactgggtccgccaggctccagggaaggggctggagtgggtctcagttatttatagcggtggtagcacatactacgcagactccgtgaagggcagattcaccatctccagagacaattccaacaacacgctgtatcttcaaatgaacagcctgagagccgaggacacggctgtgtatttctgtgcgagtgtcctcaccgagaactggtacttcgatctctggggccgtggcaccctggtcaccgtctcctcag

SEQ ID NO: 107 - DNA, 352 bp, synthetic construct
  misc_feature 1..352: note = Internal control P32
  source 1..352: mol_type = other DNA
gaggtgcaactggtggagtctgggggaggcttggtccagcctggagggtccctgatactctcctgtgcttcctctggattcaccttcagtgaccactacatggactgggtccgccaggctccagggaaggggctggagtgggttggccgtagtggaaacaaagataatcgttctaccacaagatacgccgcgtctgtggagggcagattcaccatctcaagagatgaatcgaagaacttagtatatttgcaaatgaacaacctgaaaagcgaagacacggccatatattactgtactagagataaaggtcatctctgctggggccagggaaccctggtcaccgtctcctcag

SEQ ID NO: 108 - DNA, 388 bp, synthetic construct
  misc_feature 1..388: note = Internal control P33
  source 1..388: mol_type = other DNA
gaggtgcagctggtggagtccgggggaggcttggtccagcctggggggtccctgaaactctcctgtgcagcctctgggttcaccttcagtggctctgctatgcactgggtccgccaggcttccgggaaagggctggagtgggttggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattactgtacatacgatttttggagtggttattgggtgggtgactactactactacggtatggacgtctggggccaagggaccacggtcaccgtctcctcag

SEQ ID NO: 109 - DNA, 378 bp, synthetic construct
  misc_feature 1..378: note = Internal control P34
  source 1..378: mol_type = other DNA
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagccccggagagtattatgtgccgtcagactactactactacggtatggacgtctggggccaagggaccacggtcaccgtctcctca

SEQ ID NO: 110 - DNA, 354 bp, synthetic construct
  misc_feature 1..354: note = Internal control P35
  source 1..354: mol_type = other DNA
caggtgcagatgcaggagtcgggcccaggaatggtgaagccttcggagaccctgtccctcacttgcactgtctctggtggctccatcagtagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagagatctcaggttgggggtagctgctacgatcccatacggtatggacgtctggggccaagggacc

SEQ ID NO: 111 - DNA, 394 bp, synthetic construct
  misc_feature 1..394: note = Internal control P36
  source 1..394: mol_type = other DNA
cagctgcagctgcaggagtccggctcaggactggtgaagccttcacagaccctgtccctcacctgcgctgtctctggtggctccatcagcagtggtggttactcctggagctggatccggcagccaccagggaagggcctggagtggattgggtacatctatcatagtgggagcacctactacaacccgtccctcaagagtcgagtcaccatatcagtagacaggtccaagaaccagttctccctgaagctgagctctgtgaccgccgcggacacggccgtgtattactgtgccagaggccaatacaattactatgatagtagtggttattactacgatagacagtactactttgactactggggccagggaaccctggtcaccgtctcctcag

SEQ ID NO: 112 - DNA, 381 bp, synthetic construct
  misc_feature 1..381: note = Internal control P37
  source 1..381: mol_type = other DNA
caggtgcagctgcaggagtcgggcccaggactggtgaagccttcacagaccctgtccctcacctgcactgtctctggtggctccatcagcagtggtgattactactggagttggatccgccagcccccagggaagggcctggagtggattgggtacatctattacagtgggagcacctactacaacccgtccctcaagagtcgagttaccatatcagtagacacgtccaagaaccagttctccctgaagctgagctctgtgactgccgcagacacggccgtgtattactgtgccagagaggggggggggtattacgatttttggagtggttattacggtatggacgtctggggccaagggaccacggtcaccgtctcctca

SEQ ID NO: 113 - DNA, 388 bp, synthetic construct
  misc_feature 1..388: note = Internal control P38
  source 1..388: mol_type = other DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagtgggggattactatgatagtagtggttattaccccggatcgtactactttgactactggggccagggaaccctggtcaccgtctcctcag

SEQ ID NO: 114 - DNA, 373 bp, synthetic construct
  misc_feature 1..373: note = Internal control P39
  source 1..373: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagacgcctacgctattacgatattttgactggttacccgactcttgactactggggccagggaaccctggtcaccgtctcctcag

SEQ ID NO: 115 - DNA, 385 bp, synthetic construct
  misc_feature 1..385: note = Internal control P40
  source 1..385: mol_type = other DNA
caggtgcagctgcaggagtcgggcccaggactggtgaagccttcggagaccctgtccctcacctgcgctgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgagacttaaggattacgatttttggagtggttattatacggggtatggtgcttttgatatctggggccaagggacaatggtcaccgtctcctcag

SEQ ID NO: 116 - DNA, 379 bp, synthetic construct
  misc_feature 1..379: note = Internal control P41
  source 1..379: mol_type = other DNA
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagccaaaccgggtatagcagcagctggtacacccggagctggttcgacccctggggccagggaaccctggtcaccgtctcctcag

SEQ ID NO: 117 - DNA, 391 bp, synthetic construct
  misc_feature 1..391: note = Internal control P42
  source 1..391: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagcccgcgaccccatgtattacgatttttggagtggttattataccggtatcttgggctttgactactggggccagggaaccctggtcaccgtctcctcag

SEQ ID NO: 118 - DNA, 381 bp, synthetic construct
  misc_feature 1..381: note = Internal control P43
  source 1..381: mol_type = other DNA
caggtgcagctgcaggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaaccctatattacgatttttggagtgggcctcactactacggtatggacgtctggggccaagggaccacggtcaccgtctcctca

SEQ ID NO: 119 - DNA, 359 bp, synthetic construct
  misc_feature 1..359: note = Internal control P44
  source 1..359: mol_type = other DNA
gaagtgcagctggtgcagtctggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgatcccggtagtaccagctgctatggggggagtactggggccagggaaccctggtcaccgtctcctcag

SEQ ID NO: 120 - DNA, 376 bp, synthetic construct
  misc_feature 1..376: note = Internal control P45
  source 1..376: mol_type = other DNA
gatgtgcagctggtgcagtctggagcagaggtgaaaaagcccggggagtctctaaggatctcctgtaaggcttctggacacagcttgaccgattactgggtcacctgggtgcgccagatgcccgggaaaggcctggagtggatgggggtcatctatcctgctgactctactacaacatacaacccgtccttccaaggccaggtcagtatctcagccgacaagtccatcagcaccgcctacctgcagtggagcagcctgaaggcctcggacagcgccatgtattactgtgcgagggttcgaggtggaaacagtgactggtaccaccagggctactttgactactggggccagggagccctggtcaccgtctcctcag

SEQ ID NO: 121 - DNA, 364 bp, synthetic construct
  misc_feature 1..364: note = Internal control P46
  source 1..364: mol_type = other DNA
caggtacagctgcagcagtcaggtccaggactggtgacgccctcgcagaccctctcactcacctgtgccatctccggggacagtgtctctagcaatagtgttgtttggaactggatcaggcagtccccatcgagaggccttgagtggctgggaaggacatactacaggtccaagtggtatagtgattatgcagtggctgtgagaagtcgaatatccatcaacccagacacatccaagaaccagttctctctgcagctgaactctgtgactcccgaagacacggctgtttattactgtgcaagatacattggttactactactttgaccagtgggcccagggaaccctggtcaccgtctcctcag

SEQ ID NO: 122 - DNA, 361 bp, synthetic construct
  misc_feature 1..361: note = Internal control P47
  source 1..361: mol_type = other DNA
caggtgcagctggtgcaatctgggtctgagttgaagaagcctggggcctcagtgaaggtttcctgcaaggcttctggatacaccttcactagctatgctatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgagagagcagtggctgccccagggcaactttgactactggggccagggaaccctggtcaccgtctcctcag

SEQ ID NO: 123 - DNA, 20 bp, synthetic construct
  misc_feature 1..20: note = Forward primer IGHL2-1.8_v1
  source 1..20: mol_type = other DNA
gtgtcctctccacaggtgtc

SEQ ID NO: 124 - DNA, 21 bp, synthetic construct
  misc_feature 1..21: note = Forward primer IGHL2-1.8_v2
  source 1..21: mol_type = other DNA
gtgtcctctccacaggtgtcc

SEQ ID NO: 125 - DNA, 20 bp, synthetic construct
  misc_feature 1..20: note = Forward primer IGHL2-1.8_v3
  source 1..20: mol_type = other DNA
gtcctctccacaggtgtcca

SEQ ID NO: 126 - DNA, 22 bp, synthetic construct
  misc_feature 1..22: note = Forward primer IGHL2-1.8_v4
  source 1..22: mol_type = other DNA
ctgtgtcctctccacaggtgtc

SEQ ID NO: 127 - DNA, 20 bp, synthetic construct
  misc_feature 1..20: note = Forward primer IGHL2-1.8_v5
  source 1..20: mol_type = other DNA
gtttcctctccacaggtgtc

SEQ ID NO: 128 - DNA, 20 bp, synthetic construct
  misc_feature 1..20: note = Forward primer IGHL2-1.8_v6
  source 1..20: mol_type = other DNA
gggtcctctccacaggtgtc

SEQ ID NO: 129 - DNA, 22 bp, synthetic construct
  misc_feature 1..22: note = Forward primer IGHL2-1.8_v7
  source 1..22: mol_type = other DNA
gtgtcctctccacaggtgtcca

SEQ ID NO: 130 - DNA, 20 bp, synthetic construct
  misc_feature 1..20: note = Forward primer IGHL2-1.8_v8
  source 1..20: mol_type = other DNA
gtgtcctgtccacaggtgtc

SEQ ID NO: 131 - DNA, 26 bp, synthetic construct
  misc_feature 1..26: note = Forward primer IGHL2-1.8_v10
  source 1..26: mol_type = other DNA
gtgtcctgtccacaggtgtccagtcc

SEQ ID NO: 132 - DNA, 26 bp, synthetic construct
  misc_feature 1..26: note = Forward primer IGHL2-1.8_v11
  source 1..26: mol_type = other DNA
gtgtcctstccacaggtgtccagtcc

SEQ ID NO: 133 - DNA, 25 bp, Synthetic construct
  source 1..25: mol_type = other DNA
atggagttctggctgagctgggttc

SEQ ID NO: 134 - DNA, 19 bp, Synthetic construct
  source 1..19: mol_type = other DNA
cttggggctggtcggggat